(19) United States
(12) Patent Application Publication
(10) Pub. No.: US 2025/0325491 A1
(43) Pub. Date: Oct. 23, 2025

(54) POLYVALENT MOLECULE BASED LIPID NANOPARTICLES FOR NUCLEIC ACID DELIVERY

(71) Applicant: Bio-TRIP B.V., Eindhoven (NL)

(72) Inventors: Pieter Michele FRANSEN, Boxtel (NL); Henricus Marie JANSSEN, Eindhoven (NL); Roy VAN DER MEEL, Eindhoven (NL); Egbert Willem MEIJER, Waalre (NL); Willem Jan Menno MULDER, Eindhoven (NL); Basil Frank Maria de Waal, Boxtel (NL); Stijn HOFSTRAAT, Eindhoven (NL)

(21) Appl. No.: 18/870,116

(22) PCT Filed: Jun. 5, 2023
(86) PCT No.: PCT/EP2023/064963

§ 371 Date: Nov. 27, 2024

(30) Foreign Application Priority Data
Jun. 3, 2022 (EP) 22177313

(51) Int. Cl. A61K 9/51; A61K 31/7105; A61K 31/713; C12N 15/113; C12N 15/88

(52) U.S. Cl. CPC A61K 9/5123; A61K 9/5146; A61K 9/5169; A61K 31/7105; A61K 31/713; C12N 15/113; C12N 15/88; C12N 2310/14

(57) ABSTRACT

The invention relates to nanoparticles particularly for nanoparticles suitable for the delivery of a nucleic acid to a cell. The nanoparticles comprise polyvalent molecules to stabilise the nucleic acid molecules in the nanoparticles. Particularly the polyvalent molecules have a dendrimer like structure. The invention further relates to manufacturing nanoparticles, and uses of such nanoparticles in the treatment of a disease.

### FIELD OF THE INVENTION

The invention relates to the field of nucleic acid therapeutics and provides a novel and inventive nanoparticle for the intracellular delivery of nucleic acids at a target site. The invention therefore relates to nanoparticles comprising a nucleic acid.

### BACKGROUND OF THE INVENTION

Nucleic acid therapeutics such as small antisense oligonucleotides (ASO), small interfering RNA (siRNA), messenger RNA (mRNA) and other types are a revolutionary new class of drugs that have the potential to regulate gene expression. In recent years, several nucleic acid-based drug products for in vivo applications have been approved including ASOs, N-acetylgalactosamine (GalNAc)-siRNA conjugates, lipid nanoparticles (LNP) containing siRNA or mRNA and a number of viral vectors containing plasmid DNA (pDNA). In addition, there are several nucleic acid therapeutics in late-stage clinical trials. Furthermore, several genetically engineered ex vivo cell therapy drug products have been approved.

The therapeutic application of nucleic acids following parenteral administration is challenging. Although nucleic acid types vary in size and physicochemical properties, their common features include their large, macromolecular size and negative charge. As a result, upon systemic administration, nucleic acids are rapidly cleared from the circulation due to kidney filtration and nuclease degradation. In addition, nucleic acid therapeutics act intracellularly but cannot readily pass cellular membranes. Finally, administration of exogenous nucleic acids provokes an immune response. While this can be advantageous (e.g., for vaccine development), usually this contributes to nucleic acids' rapid clearance and adverse effects.

To overcome these challenges, all nucleic acid therapeutics rely on chemical modifications and/or nanotechnology-based delivery systems. All approved nucleic acid therapeutics are dependent on chemical modifications and/or nanotechnology platforms to facilitate their intracellular delivery and subsequently induce therapeutic effects following parenteral administration:
1) ASOs are heavily chemically modified to increase their stability, reduce immunostimulatory effects and increase their efficacy. They are administered subcutaneously to target hepatocytes or intrathecally to target cells in the central nervous system.
2) GalNAc-siRNA conjugates are similarly modified as ASOs and are administered subcutaneously. The GalNAc moiety ensures asialoglycoprotein receptor-mediated uptake in hepatocytes.
3) Lipid nanoparticles (LNPs) are ˜50-100 nm in diameter and can be administered systemically, intradermally, or intramuscularly. Following systemic administration, LNPs efficiently accumulate in hepatocytes providing opportunities for gene silencing (siRNA) or protein production (mRNA). Following intradermal or intramuscular administration, LNPs are taken up by immune cells such as antigen presenting cells which can be exploited for vaccine purposes. LNPs are the current golden standard for mRNA therapeutics and will likely also become the standard delivery platform for gene editing applications in vivo. However, LNPs contain synthetic (non-natural) polyethylene glycol (PEG)-conjugated lipids which have been associated with hypersensitivity reactions and or anaphylaxis. In addition, current LNP systems accumulate predominantly in the liver following intravenous administration.
4) Viral delivery systems such as adenoviruses, lentiviruses, or adeno-associated virus (AAV) vectors are effective vehicles to deliver DNA. Viral vectors are characterized by their limited payload capacity and immunogenicity. However, in immune-privileged tissues such as the eye, viral vectors constitute the current golden standard for nucleic acid therapeutics. Viral vectors are extensively used for ex vivo therapeutics (e.g., CAR T) or are administered intravenously to target cells in the liver, intravitreally/subretinally to target cells in the retina or intramuscularly for vaccine purposes.

With the exception of viral vector- or LNP-mRNA-based vaccines, the majority of approved nucleic acid therapeutics is developed for other indications than immunotherapy. Delivering therapeutic nucleic acids to the myeloid compartment therefore remains a challenge. Furthermore, chemical modifications of nucleic acid molecules or viral delivery inherently have the risk of unwanted activation of the immune system, resulting in degradation or clearance of the nucleic acid therapeutics, or undesired immune responses.

For example, nanoparticles carrying nucleic acids have been described for example in WO2009127060A1 which describes the use of cationic lipids combined with non-cationic lipids and nucleic acids. The cationic lipids neutralize the nucleic acid, allowing the formation of nanoparticles which may be used for non-targeted delivery of the nucleic acids in a subject. A drawback of these nanoparticles is that they are not capable of targeting the myeloid compartment.

Other systems, for example in WO2019103998A2, describe nanobiologics that are able to target the myeloid compartment, the nanobiologics comprising phospholipids and apo A1 and a small molecule drug. The drawback of these nanobiologics is that due to their hydrophobic core they do not allow the incorporation of polar structures such as nucleic acids, e.g. DNA and RNA.

In WO2017048789A1 dendrimer materials are described with a limited number of ionizable groups per molecule and with non-natural sulfide groups that are prone to in vivo oxidation. In the preparation of these materials, a multifunctional molecule is reacted with a bifunctional molecule to arrive at an intermediate that accordingly may be hard to attain in pure (non-crosslinked) form.

Therefore, there is a need for improved and alternative delivery systems for therapeutic nucleic acids to the myeloid compartment.

The above problems, among others, are solved by the invention as defined in the appended claims.

### SUMMARY OF THE INVENTION

The current invention constitutes nanoparticle platform technology for nucleic acid therapeutic targeting and/or delivery, and more particularly for nucleic acid therapeutic targeting and/or delivery to the myeloid cell compartment. The nanoparticles as taught herein are (phospho)lipid-based nanoparticles stabilised by an apolipoprotein stabiliser (or by a derivative of an apolipoprotein, or by a mimic or a mimic derivative of an apolipoprotein). In circulation, the stabiliser and (phospho)lipids shield and/or protect the nucleic acid payload, and thereby prevent it from degradation and rapid clearance. At the same time, the nanoparticles reduce nucleic acid therapeutics' immunostimulatory-related adverse effects by limiting unwanted interactions with components in the blood, such as limiting unwanted interactions of the nucleic acid payload with components in the blood. The apolipoprotein stabiliser also acts as a targeting moiety as it is capable of directing the nanoparticle to the myeloid cell compartment. Accordingly, the invention enables efficient nucleic acid therapeutics delivery to the myeloid cell compartment in lymphoid organs, such as for example the bone marrow and the spleen, for effective immunotherapy.

The nanoparticles as taught herein further also comprise polyvalent molecules. The polyvalent molecule has multiple positively ionizable and/or cationic groups that can efficiently bind and capture (or complex) nucleic acids. The polyvalent molecules of the invention bind stronger to nucleic acids than, for example, monovalent amphiphilic molecules. Accordingly, the nucleic acids are better bound and/or retained within the nanoparticles as taught herein. In addition, the polyvalent molecules of the invention may also interact with the apolipoprotein stabiliser (or by a derivative of an apolipoprotein, or by a mimic or a mimic derivative of an apolipoprotein), as this protein has an overall negative charge. As a result, the apolipoprotein (or by a derivative of an apolipoprotein, or by a mimic or a mimic derivative of an apolipoprotein) becomes better integrated in the nanoparticle of the invention. Hence, in circulation, e.g. in the blood of a subject, the nanoparticle of the present invention will release very little or none of the apolipoprotein component from the nanoparticle.

The invention relates in particular to nanoparticles comprising a polyvalent molecule such as but not limited to a dendrimer. Furthermore, the invention relates in particular to nanoparticles comprising a stabiliser material such as, but not limited to, an apolipoprotein, an apolipoprotein derivative, an apolipoprotein mimetic or an apolipoprotein mimetic derivative. The invention further relates to methods of treatment using the nanoparticle, for example in the treatment of a disease by stimulating or inhibiting an innate immune response. The invention further relates to an in vivo, in vitro or ex vivo method for introducing a nucleic acid in a cell using the nanoparticles.

Accordingly, in a first aspect, the invention relates to a nanoparticle comprising a core and an outer layer, wherein the core comprises:
a nucleic acid;
a polyvalent molecule;
and wherein the outer layer comprises:
an apolipoprotein, an apolipoprotein derivative, an apolipoprotein mimetic and/or an apolipoprotein mimetic derivative;
a phospholipid,
a sterol; and optionally,
a filler molecule,
wherein the polyvalent molecule has formula (I):

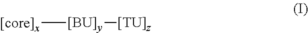

wherein the core (i.e. core of the polyvalent molecule) is a nitrogen or is a C₁-C₁₈ linear, branched, or cyclic group that contains 1 to 15 nitrogen heteroatoms and that optionally contains 1 to 4 oxygen heteroatoms; and
wherein x represents the number of connections from the core to the branching units BU (or to the terminal units TU, when y=0), where x is 1, 2, 3, 4, 5, 6, 7, 8, 9, 10 or 12, and where these connections are all made from nitrogen atoms of the core to carbon atoms of the branching unit BU (or of the terminal unit TU); and
wherein the branching unit BU has formula (IIa), (IIb), (IIc) or (IId):

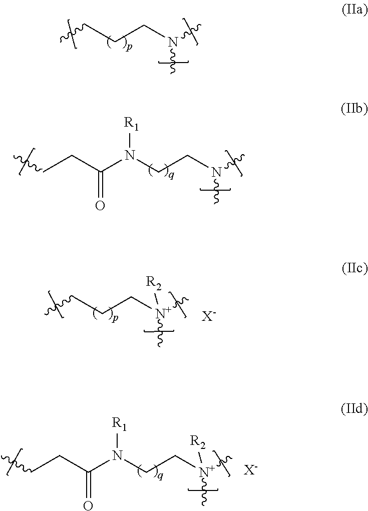

wherein (IIa) represents an n-alkylene-nitrogen spacer that is connected at the CH₂-end to the core or to a BU that is closer to the core, and that is connected at the N-end to two terminal units (TUs) or to two branching units (BUs) that are closer to the TUs, where the connections are given in wavy bonds, and wherein p is 1, 2, 3 or 4; and
wherein (IIb) represents an amide containing n-alkylene-nitrogen spacer that is connected at the CH₂-end to the core or to a BU that is closer to the core, and that is connected at the N-end to two terminal units (TUs) or to two branching units (BUs) that are closer to the TUs, where the connections are given in wavy bonds, wherein q is 1, 2, 3, 4 or 5, and wherein R₁ is a hydrogen, a methyl, an ethyl, an n-propyl or an iso-propyl group; and
wherein (IIc) and (IId) are defined as (IIa) and (IIb), respectively, and wherein R₂ is a methyl, ethyl, n-propyl, benzyl, acetamide (—CH₂—C(O)NH₂) or 2-hydroxy-ethylene group, and wherein X⁻ is the counter anion to the quaternary amine cation moiety; and
wherein y represents the specific and discrete generation number of the polyvalent molecule, where this number indicates how many successive BU layers are incorporated in the polyvalent molecule, as counted from traveling from the inner core to the outer TU groups, and where y is 0, 1, 2, 3, 4 or 5; and
wherein for each separate generational layer the BU can independently be selected from formulas (IIa), (IIb), (IIc) or (IId), i.e. the first BU emanating from the core, representing the first generational layer BU, can be different from the second BU emanating further away from the core (representing the second generational layer BU), and this one may be different from the third, the fourth and the fifth BU emanating further away from the core; and
wherein the terminal units (TU) are individually and independently chosen from a hydrogen and a C₁-C₃₀ alkyl, aryl, arylene-alkyl or alkylene-aryl group that optionally contains 1 to 8 heteroatoms that are individually and independently selected from the group consisting of O and N, with the proviso that not all TUs in the polyvalent molecule are hydrogens; and
wherein z represents the total number of TU groups that are attached to the polyvalent molecule, where z is 1 to 128; and
wherein the polyvalent molecule as according to formula (I) is polyvalent in positively ionizable and/or cationic groups, where the cumulative number of positively ionizable and cationic groups in the polyvalent molecule is 2 or higher.

In a second aspect, the invention relates to a composition comprising the nanoparticle according to the first aspect of the invention and a physiologically acceptable carrier, preferably wherein the composition is a pharmaceutical composition.

In a third aspect the invention relates to a nanoparticle according to the first aspect of the invention, or the composition according to the second aspect of the invention for use as a medicament.

In a fourth aspect the invention relates to a nanoparticle according to the first aspect of the invention, or the composition according to the second aspect of the invention for use in the treatment of a disease by stimulating or inhibiting an innate immune response, preferably wherein said disease is a cancer, a cardiovascular disease, an autoimmune disorder, or a xenograft rejection.

In a fifth aspect the invention relates to a method for producing a nanoparticle, comprising the step of:
a) mixing, preferably rapid mixing, of (lipid) components in organic solvent with a nucleic acid in an aqueous buffer to produce nanoparticles, wherein the (lipid) components comprise a phospholipid, a sterol, a polyvalent molecule, and optionally a filler material (e.g. a tri-glyceride); and wherein the aqueous buffer has a pH of 5.5 or lower, preferably 5.0 or lower; and
b) mixing, preferably rapid mixing, of the lipid nanoparticles produced under a. with an apolipoprotein, an apolipoprotein derivative, an apolipoprotein mimetic and/or an apolipoprotein mimetic derivative, to produce the nanoparticle of the invention at a pH between 5.5 and 9.0, preferably at a pH between 6.0 and 8.0, more preferably at a pH between 6.5 and 8.0.

In a sixth aspect the invention relates to an in vitro or ex vivo method for introducing a nucleic acid in a cell, the method comprising contacting the nanoparticle according to the first aspect of the invention or the composition according to the second aspect of the invention with the cell.

In a seventh aspect the invention relates to an in vivo method for introducing a nucleic acid in a cell, the method comprising contacting the nanoparticle according to the first aspect of the invention or the composition according to the second aspect of the invention with the cell.

In an eight aspect the invention relates to a method for the in vivo delivery of a nucleic acid, the method comprising administering the nanoparticle according to the first aspect of the invention or the composition according to the second aspect of the invention to a subject.

In a nineth aspect the invention relates to a method for treating a disease or disorder in a subject in need thereof by stimulating or inhibiting an innate immune response, the method comprising administering a therapeutically effective amount of the nanoparticle according to the first aspect of the invention or the composition according to the second aspect of the invention to the subject.

### BRIEF DESCRIPTION OF THE FIGURES

FIG. 1. Exemplary polyvalent molecule structures, with their respective codes.

FIG. 2. Exemplary polyvalent molecule structures, with their respective codes.

FIG. 3. Exemplary polyvalent molecule structures, with their respective codes.

FIG. 4. Reference molecule structures of DLin-MC3-DMA and ALC-0315.

FIG. 5. Schematic representation of the production process of exemplary RNA containing apolipoprotein nanoparticles (aNP). The lipidic components are solubilized in organic solvent and are mixed with RNA (for example siRNA) solubilized in buffered water at a low pH (for example pH 4). The formed particles are dialyzed in PBS pH 7.4 and then mixed with apolipoprotein (for example apolipoprotein A1). Finally, the particle solutions are filtered and centrifuged. The reference LNP nanoparticles contain the ionizable cationic lipid DLin-MC3-DMA and do not contain tri-caprylin (TG) nor apolipoprotein A1 (for LNPs the second mixing step is not required). The reference aNPs (containing DLin-MC3-DMA) and the exemplary aNPs (containing polyvalent ionizable molecules) according to the invention do not contain polyethylene glycol (PEG)-conjugated lipids (aNPs are apolipoprotein nanoparticles). See Table S3 for further details.

FIG. 6. siRNA retention values for prepared nanoparticles as determined by Ribogreen assay. The dark-grey columns show data for the reference nanoparticles LNP (containing the ionizable cationic lipid DLin-MC3-DMA) and aNP (containing DLin-MC3-DMA), while the light-grey columns show data for the exemplary aNPs (comprising polyvalent ionizable materials A1, A2, A3, A6, A7, A8, A11, A14, B2, D4, G2, G6, G7, G8, G9, H1 or F1) according to the invention. Each circle represents an individual repeat experiment.

FIG. 7. Cryo-TEM images of siRNA-aNPs containing A2 (A) and A1 (B). On the left an overview of the particles (white scale bars are 100 nm) are shown, while on the right magnifications are given. Spherical particles are found for both A2 and A1. For A1 it seems that the aNPs have (multiple) outer layer(s).

FIG. 8. Cryo-TEM images of siRNA-aNPs containing A6 (C) and A14 (D). On the left an overview of the particles (white scale bars are 100 nm) are shown, while on the right magnifications are given. Spherical particles are found for both A6 and A14. For A6 and A14 it seems that the aNPs have (multiple) outer layer(s).

FIG. 9. Comparing Cryo-TEM images (A) with immuno-gold staining TEM images (B) of the same exemplary apolipoprotein nanoparticles (aNP) according to the invention containing A6. In both pictures siRNA-aNPs of about 50 nm in diameter are visible. Immuno-gold staining TEM imaging (IGS-TEM) has been implemented to localize apolipoprotein A1. In this method, a primary antibody binds to apolipoprotein A1 on the nanoparticles, followed by a bridging antibody (IGg) and protein A-coated gold nanoparticles (10 nm in diameter). In (B) the 10-nanometer gold nanoparticles appear as black spheres, where these are mostly located at the surface of the aNPs.

FIG. 10. Cytotoxicity measurements. MTS assay on RAW 264.7 cell line, with exposure to siRNA-aNPs as taught herein. Increasing amounts of the polyvalent molecules A1, A2 and A6 do not exercise cytotoxicity.

FIG. 11. Dose response curve for siRNA-aNPs as taught herein (containing A1 or A2 ionizable polyvalent molecules) and for reference aNPs (containing either DLin-MC3-DMA or ALC-0315 ionizable cationic lipids), as carried out on RAW 264.7 cell line. Each point represents an individual repeat for a certain concentration. LNP particles with DLin-MC3-DMA ionizable cationic lipid show an IC50 value of about 30 nM (curve not shown). The aNPs as taught herein show a higher potency than the reference aNPs or LNPs containing monovalent ionizable cationic lipids.

FIG. 12. Dual-luminescence gene silencing reporter assay. Data show the silencing (in grey) and lack of silencing (in white) of nanoparticles containing firefly luciferase siRNA and non-specific siRNA, respectively. Each point represents and individual repeat (the average of three technical repeats). The data show that the exemplary aNPs as taught herein mostly outperform the reference LNP and aNP particles.

FIG. 13. siRNA retention values for prepared A2 aNPs as taught herein as determined by Ribogreen assay. Variations in N/P, in tri-caprylin (TG) content, in cholesterol content, and in type of phospholipid used, lead to nanoparticles with (in most cases) a high siRNA retention. Each circle represents an individual repeat experiment. Accordingly, aNPs can be prepared with a variety in composition.

FIG. 14. Luminescence intensity measured after transfection of RAW264.7 macrophage cells with 100 ng Firefly luciferase mRNA per well formulated in nanoparticles. The exemplary aNPs according to the invention contain polyvalent material A2. Two controls are shown, an LNP and an aNP, both containing ALC-0315 as ionizable cationic material. Data are represented as mean+SD from one experiment with three technical repeats. The data show that the exemplary aNPs as taught herein outperform the reference LNP and aNP particles.

FIG. 15. Cryo-TEM image of mRNA-loaded aNPs containing ionizable material A2. These aNPs have been used for in vivo experiments. A scale bar of 50 nm is given. Spherical particles with seemingly (multiple) outer layer(s) are found.

FIG. 16. Luminescence intensity measured from single cell suspensions prepared from mice that were injected with 0.5 mg/kg Firefly luciferase mRNA (TriLink, CleanCap 5 moU) loaded in an aNP containing polyvalent material A2. Measuring took place 16 hours after bolus injection. Data are presented as mean±SD from two animals. The data show that the exemplary aNPs as taught herein target spleen and bone marrow tissue efficiently.

FIG. 17. Exemplary polyvalent molecule structures, with their respective codes.

### DETAILED DESCRIPTION OF THE INVENTION

For purposes of the present invention, the following terms are defined below.

As used herein, the singular form terms “A,” “an,” and “the” include plural referents unless the content clearly dictates otherwise. Thus, for example, reference to “a cell” includes a combination of two or more cells, and the like.

As used herein, the term “and/or” refers to a situation wherein one or more of the stated cases may occur, alone or in combination with at least one of the stated cases, up to with all of the stated cases.

As used herein, the term “antigen” refers to a substance to which a binding portion of an antibody may bind. The specific immunoreactive sites within the antigen are known as “epitopes” (or antigenic determinants). A target for an antibody, or antigen-binding portion thereof, may comprise an antigen, such as is defined herein.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . −10, −11, etc.

As used herein, the word “comprise” or variations thereof such as “comprises” or “comprising” will be understood to include a stated element, integer or step, or group of elements, integers or steps, but not to exclude any other element, integer or steps, or groups of elements, integers or steps. The verb “comprising” includes the verbs “essentially consisting of” and “consisting of”.

As used herein, the term “conventional techniques” refers to a situation wherein the methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

When used herein, a nanoparticle refers to a small particle, e.g. in the range of 10 to 200 nm diameter which may be used to deliver a payload to a target, e.g. an organ or cell in a subject.

As used herein, the term “identity” refers to a measure of the identity of nucleotide sequences or amino acid sequences. In general, the sequences are aligned so that the highest order match is obtained. “Identity” per se has an art-recognized meaning and can be calculated using published techniques. See, e.g.: (Computational Molecular Biology, Lesk, A. M., ED., Oxford University Press, New York, 1988; Biocomputing: Informatics And Genome Projects, Smith, D. W., ED., Academic Press, New York, 1993; Computer Analysis Of Sequence Data, Part I, Griffin, A. M., And Griffin, H. G., EDS., Humana Press, New Jersey, 1994; Sequence Analysis In Molecular Biology, Von Heinje, G., Academic Press, 1987; and Sequence Analysis Primer; Gribskov, M. and Devereux, J., eds., M Stockton Press, New York, 1991). While there exist a number of methods to measure identity between two nucleotide sequences or amino acid sequences, the term “identity” is well known to skilled artisans (Carillo, H., and Lipton, D., SIAM J. Applied Math (1988) 48:1073). Methods commonly employed to determine identity or similarity between two sequences include, but are not limited to, those disclosed in Guide To Huge Computers, Martin J. Bishop, ed., Academic Press, San Diego, 1994, and Carillo, H., and Lipton, D., Siam J. Applied Math (1988) 48:1073. Methods to determine identity and similarity are codified in computer programs. Preferred computer program methods to determine identity and similarity between two sequences include, but are not limited to, GCS program package (Devereux, J., et al., Nucleic Acids Research (1984) 12(1):387), BLASTP, BLASTN, FASTA (Atschul, S. F. et al., J. Molec. Biol. (1990) 215:403).

As an illustration, by a polynucleotide having a nucleotide sequence having at least, for example, 95% “identity” to a reference nucleotide sequence encoding a polypeptide of a certain sequence, it is intended that the nucleotide sequence of the polynucleotide is identical to the reference sequence except that the polynucleotide sequence may include up to five point mutations per each 100 nucleotides of the reference amino acid sequence. In other words, to obtain a polynucleotide having a nucleotide sequence at least 95% identical to a reference nucleotide sequence, up to 5% of the nucleotides in the reference sequence may be deleted and/or substituted with another nucleotide, and/or a number of nucleotides up to 5% of the total nucleotides in the reference sequence may be inserted into the reference sequence. These mutations of the reference sequence may occur at the 5′ or 3′ terminal positions of the reference nucleotide sequence, or anywhere between those terminal positions, interspersed either individually among nucleotides in the reference sequence or in one or more contiguous groups within the reference sequence.

Similarly, by a polypeptide having an amino acid sequence having at least, for example, 95% “identity” to a reference amino acid sequence of SEQ ID NO: X is intended that the amino acid sequence of the polypeptide is identical to the reference sequence except that the amino acid sequence may include up to five amino acid alterations per each 100 amino acids of the reference amino acid of SEQ ID NO: X. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As used herein, the term “in vitro” refers to experimentation or measurements conducted using components of an organism that have been isolated from their natural conditions.

As used herein, the term “ex vivo” refers to experimentation or measurements done in or on tissue from an organism in an external environment with minimal alteration of natural condition.

As used herein, the term “nucleic acid”, “nucleic acid molecule” and “polynucleotide” is intended to include DNA molecules and RNA molecules, as well as locked nucleic acid (LNA), bridged nucleic acid (BNA), morpholino or peptide nucleic acid (PNA). A nucleic acid (molecule) may be any nucleic acid (molecule), it may be single-stranded or double-stranded.

As used herein, the terms “sequence” when referring to nucleotides, or “nucleic acid sequence”, “nucleotide sequence” or “polynucleotide sequence” refer to the order of nucleotides of, or within, a nucleic acid and/or polynucleotide. Within the context of the current invention a first nucleic acid sequence may be comprised within or overlap with a further nucleic acid sequence.

As used herein, the term “subject” or “individual” or “animal” or “patient” or “mammal,” used interchangeably, refer to any subject, particularly a mammalian subject, for whom diagnosis, prognosis, or therapy is desired. Mammalian subjects include humans, domestic animals, farm animals, and zoo-, sports-, or pet-animals such as dogs, cats, guinea pigs, rabbits, rats, mice, horses, cattle, cows, bears, and so on. As defined herein a subject may be alive or dead. Samples can be taken from a subject post-mortem, i.e. after death, and/or samples can be taken from a living subject.

As used herein, terms “treatment”, “treating”, “palliating”, “alleviating” or “ameliorating”, used interchangeably, refer to an approach for obtaining beneficial or desired results including, but not limited to, therapeutic benefit. By therapeutic benefit is meant eradication or amelioration or reduction (or delay) of progress of the underlying disease being treated. Also, a therapeutic benefit is achieved with the eradication or amelioration or reduction (or delay) of progress of one or more of the physiological symptoms associated with the underlying disease such that an improvement or slowing down or reduction of decline is observed in the patient, notwithstanding that the patient can still be afflicted with the underlying disease.

As used herein the term “vector” refers to a nucleic acid molecule capable of transporting another nucleic acid to which the nucleic acid molecule capable of transporting has been linked. One type of vector is a “plasmid”, which refers to a circular double stranded DNA loop into which additional DNA segments may be ligated. The term “vector” may also refer to the viral particle (i.e. viral vector) which contains the nucleic acid of interest.

When used herein the term “payload” in general refers to a substance to be included in a particle and delivered at a target site. When referring to the nanoparticles of the invention, the term “payload” refers to the nucleic acid, preferably in combination with the polyvalent molecule.

When used herein, the term “targeting”, when referring to targeting a cell (e.g. a target cell such as but not limited to a myeloid cell) or targeting a tissue or organ should be understood to mean bring in proximity of the intended cell, organ or tissue, or to enrich in the proximity of the intended cell, organ or tissue. This implies that when targeting an intended cell, organ or tissue, on average more nanoparticle are in proximity of the intended cell, organ or tissue as can be expected based on random or natural distribution of the particle. In proximity herein means being located such that the nanoparticle can interact with the cell (or tissue or organ) to deliver its payload (nucleic acid).

When used herein, the term myeloid cell refers to blood cells that are derived from a common progenitor cell for megakaryocytes, granulocytes, monocytes, erythrocytes. Myeloid cells are a major cellular compartment of the immune system comprising monocytes, dendritic cells, tissue macrophages, and granulocytes. The term myeloid compartment, when used herein, refers to the totality of myeloid cells in an organism.

Alkyl and alkylene groups may be linear, branched, or cyclic. Alkyl and alkylene groups comprise 1 to 36 carbon atoms, preferably 1 to 18 carbons. Alkyl and alkylene groups optionally comprise one or more double bonds (C═C), in which case these groups are unsaturated. Alkyl and alkylene groups optionally comprise heteroatoms selected from the group consisting of O, N, P, S and F, preferably 1-8 heteroatoms selected from the group consisting of O and N.

Aryl and arylene groups comprise 6 to 24 carbon atoms, while alkylenearyl and arylenealkyl groups comprise 7 to 25 carbon atoms. Aryl, arylene, alkylenearyl and arylenealkyl groups optionally comprise heteroatoms selected from the group consisting of O, N, P, S and F, preferably 1-8 heteroatoms selected from the group consisting of O and N.

Ester, amide, urethane, urea, carbonate, carboxylic acid, ketone, aldehyde, ether and alcohol groups are defined hereunder, where Rx represents a hydrogen atom or a cyclic, linear or branched alkyl or alkylene group. In groups that contain more than one Rx element, then these elements can be independently selected. An ester (functional) group or moiety as indicated in this document is to be understood as a group according to the formula: —C(O)—O—. An amide (functional) group or moiety as indicated in this document is to be understood as a group according to the formula: —NRₓ—C(O)—. A urethane (functional) group or moiety as indicated in this document is to be understood as a group according to the formula: —NRₓ—C(O)—O—. A urea (functional) group or moiety as indicated in this document is to be understood as a group according to the formula: —NRₓ—C(O)—NRₓ—. A carbonate (functional) group or moiety as indicated in this document is to be understood as a group according to the formula: —O—C(O)—O—. A carboxylic acid (functional) group or moiety as indicated in this document is to be understood as a moiety or group according to the formula: —C(O)OH. A ketone (functional) group or moiety as indicated in this document is to be understood as a group according to the formula: —C(O)—. An aldehyde (functional) group or moiety as indicated in this document is to be understood as a group according to the formula: —C(O)H. An ether (functional) group or moiety as indicated in this document is to be understood as a group according to the formula: —O—. An alcohol (or hydroxy) functional group or moiety as indicated in this document is to be understood as a group according to the formula: —OH.

The section headings as used herein are for organizational purposes only and are not to be construed as limiting the subject matter described.

Various terms relating to the methods, compositions, uses and other aspects of the present invention are used throughout the specification and claims. Such terms are to be given their ordinary meaning in the art to which the invention relates, unless otherwise indicated. Other specifically defined terms are to be construed in a manner consistent with the definition as provided herein. The preferred materials and methods are described herein, although any methods and materials similar or equivalent to those described herein can be used in the practice for testing of the present invention.

Unless otherwise defined, all technical and scientific terms used herein have the same meaning as commonly understood by a person of ordinary skill in the art.

The current invention constitutes nanoparticle platform technology for nucleic acid therapeutic targeting and/or delivery to the myeloid cell compartment The nanoparticles described herein are (phospho)lipid-based nanoparticles stabilised by an apolipoprotein (or by a derivative of an apolipoprotein, or by a mimic or a mimic derivative of an apolipoprotein), also referred to herein as apolipoprotein stabiliser. In circulation, the stabiliser and/or (phospho)lipids shield and/or protect the nucleic acid therapeutic payload, and thereby prevent it from degradation and rapid clearance. At the same time, the nanoparticles reduce nucleic acid therapeutics' immunostimulatory-related adverse effects by limiting unwanted interactions with components in the blood, such as limiting unwanted interactions of the nucleic acid payload with components in the blood. The apolipoprotein stabiliser also acts as a targeting moiety as it is capable of directing the nanoparticle to the myeloid cell compartment. Accordingly, the invention enables efficient nucleic acid therapeutics delivery to the myeloid cell compartment in lymphoid organs, such as for example the bone marrow and the spleen, for effective immunotherapy. In particular embodiments, the nanoparticles as taught herein are used to target or deliver to a myeloid cell, including all blood cells that are derived from a progenitor cell for granulocytes, monocytes, erythrocytes, or platelets. In particular embodiments, the nanoparticles as taught herein are used to target or deliver to monocytes, dendritic cells, tissue macrophages, or granulocytes.

Nanoparticles as described herein are lipid-based nano-sized formulations (diameter of particles ˜10-300 nm). Without being bound to theory, present inventors believe that the particles have a core comprised of the nucleic acid interacting with the polyvalent molecule, and an outer surface with incorporated stabiliser protein(s), preferably an apolipoprotein, an apolipoprotein derivative, an apolipoprotein mimetic and/or an apolipoprotein mimetic derivative. The core is relatively hydrophobic due to the hydrophobic moieties of the polyvalent molecule. The outer surface largely consists of a layer of phospholipids with incorporated stabiliser proteins, preferably apolipoprotein stabiliser, and further comprises sterols such as for example cholesterol. The hydrophilic heads of the phospholipids face the outside aqueous environment while the hydrophobic tails point inwards towards the core of the nanoparticle. Possibly, the nanoparticles contain multiple layers, but in any case the nanoparticles have nucleic acids bound and buried inside the particle by the hydrophobic polyvalent molecule, and have an outer protective surface layer with phospholipids, sterols and the stabiliser protein, preferably an apolipoprotein, an apolipoprotein derivative, an apolipoprotein mimetic and/or an apolipoprotein mimetic derivative. Therefore, the nanoparticles described herein are distinct from nanoparticles described in the art, for example nanoparticles that are vesicle-like and comprise a lipid bilayer surrounding an aqueous core. The apolipoprotein decorated nanoparticles of this invention are also distinct from nanoparticles described in the art, for example nanoparticles that are solely stabilised by synthetic (non-natural) hydrophilic polymers, such as for example poly-ethylene-glycols (PEGs) or derivatives thereof. Notably, previously reported nanoparticles typically do not allow to target a nucleic acid cargo to specific cells, while the nanoparticles as taught herein allow to target a nucleic acid cargo to the myeloid cell compartment, as described elsewhere herein.

Phospholipids in the nanoparticle formulation, due to their amphiphilic character, accumulate at the interface between the hydrophobic core and the aqueous solvent, effectively forming a lipid monolayer (or a lipid multilayer) surface barrier. For biological uses, single or multiple phospholipid types may be used, because of their inherent biocompatibility and nett neutral charge. Optionally, mole percentages of charged (phospho)lipids may be added to give the entire formulation a specific charged character (1-95 mol %, or less, for example 1-20 mol % or 1-10 mol % or 1-5 mol %, where these mole percentages are relative to the total amount of employed phospholipid).

The lipid nanoparticles are engineered to complex within its structure nucleic acids, which are hydrophilic in nature. Accordingly, helper molecules are needed to draw the nucleic acids into the hydrophobic nanoparticle core. To this end a polyvalent molecule is included in the nanoparticle. The polyvalent molecule comprises two or more positively ionizable and/or cationic groups. The positively ionizable groups become cationic groups at a lower pH by protonation. A cationic group can bind with the anionic phosphate groups in the sugar phosphate backbone of a nucleic acid payload via ionic interactions. The hydrophobic part of the polyvalent molecule forms a shell around the hydrophilic nucleic acid molecule. This shell can then interact with the particle outer layer that is composed of phospholipids, sterols and the protein stabiliser (e.g. an apolipoprotein stabiliser).

By employing polyvalent positively ionizable and/or cationic molecules, instead of monovalent amphiphilic molecules, the binding with nucleic acids becomes stronger. As a result thereof, the nucleic acids are better bound and retained within the nanoparticles as taught herein. In addition, the polyvalent molecules may also interact with the apolipoprotein stabiliser as this protein has an overall negative charge, such as described in Sparks D L, Lund-Katz S, Phillips M C. The charge and structural stability of apolipoprotein A-1 in discoidal and spherical recombinant high density lipoprotein particles. J Biol Chem. 1992 Dec. 25; 267(36):25839-47. PMID: 1464598. As a result, the apolipoprotein becomes better attached to and captured within the nanoparticle of the invention. Finally, transfection of the nucleic acid into the targeted cells may be aided by the presence of the polyvalent molecules, as these can efficiently bind and interact with multiple phospholipids at a (myeloid) cell barrier, thus creating a gap for the nucleic acids to enter.

Besides nucleic acids, polyvalent molecules, phospholipids, sterols, and the stabiliser (which optionally has a targeting ability such as the ability to target the myeloid cell compartment), additional hydrophobic filler molecules can be included in the nanoparticle formulations as taught herein. Their main application is to alter nanoparticle physicochemical properties or improve stability. The filler molecules reside (mainly) in the core of the nanoparticle. The filler molecules are preferably biocompatible, and non-limiting examples include glyceride lipid molecules (lipid di-glycerides or tri-glycerides), sterol esters and esters of fatty acids.

Nanoparticles containing therapeutic nucleic acids are expected to precisely regulate gene expression in the myeloid cell compartment thereby modulating the immune response. A major advantage of the nanoparticle platform technology as taught herein is the possibility to exchange the nucleic acid payload without altering the formulation's biological behavior and interactions. Nanoparticles containing therapeutic nucleic acids as taught herein can therefore be implemented as immunotherapies that promote the immune response e.g. to treat cancer or infectious diseases, or to dampen the immune response e.g. to treat autoimmune diseases or during organ transplantation.

Therefore, in a first aspect, the invention relates to a nanoparticle comprising, consisting essentially of or consisting of:
a nucleic acid;
a polyvalent molecule;
a stabiliser, preferably an apolipoprotein, an apolipoprotein derivative, an apolipoprotein mimetic and/or an apolipoprotein mimetic derivative, preferably the the stabiliser is also a targeting component as it is capable of directing the nanoparticle to specific cells inside the body (i.e. the myeloid compartment);
a phospholipid,
a sterol; and optionally,
a filler molecule,
wherein the polyvalent molecule has formula (I):

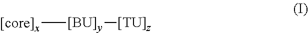

wherein the polyvalent molecule has a dendrimer(-like) branched architecture that is composed of a core that is attached by x-number of connections to the (optional) branching units (BUs) (i.e. the most inner BUs) or to the terminal units (TUs) (such as in case the polyvalent molecule does not have any BUs), and
wherein the BUs are layered in y-number of generational layers, with y is 0, 1, 2, 3, 4 or 5, and
wherein the polyvalent molecule contains z number of terminal units (TUs), preferably TUs that are connected to the core (in case y=0) or to the most outer BU units (in case y=1, 2, 3, 4 or 5), and
wherein the polyvalent molecule is polyvalent in positively ionizable and/or cationic groups, where the cumulative number of these groups in the polyvalent molecule is at least 2.

In particular embodiments, the nanoparticle comprises a core and an outer layer, wherein the core comprises, essentially consists of or consists of:
a nucleic acid;
a polyvalent molecule;
and wherein the outer layer comprises, essentially consists of or consists of:
a stabiliser, preferably an apolipoprotein, an apolipoprotein derivative, an apolipoprotein mimetic and/or an apolipoprotein mimetic derivative;
a phospholipid;
a sterol; and optionally
a filler molecule,
wherein the polyvalent molecule has formula (I):

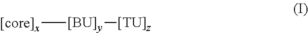

wherein the core is a nitrogen or is a C₁-C₁₈ linear, branched or cyclic group that contains 1 to 15 nitrogen heteroatoms and that optionally contains 1 to 4 oxygen heteroatoms; and
wherein x represents the number of connections from the core to the branching units BU (i.e. the inner most branching units BU), or to the terminal units TU when y=0, where x is 1, 2, 3, 4, 5, 6, 7, 8, 9, 10 or 12, and where these connections are all made from nitrogen atoms of the core to carbon atoms of the branching unit BU (i.e. the inner most branching units BU) or of the terminal unit TU, such as when y=0; and
wherein the branching unit BU has formula (IIa), (IIb), (IIc) or (IId):

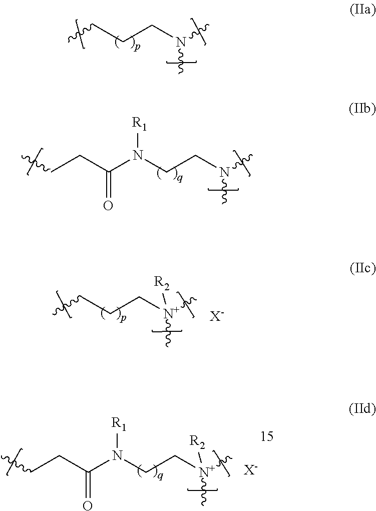

wherein (IIa) represents an n-alkylene-nitrogen spacer that is connected at the CH₂-end to the core or to a BU that is closer to the core, and that is connected at the N-end to two terminal units (TUs) or to two branching units (BUs) that are closer to the TUs, where the connections are given in wavy bonds, and wherein p is 1, 2, 3 or 4; and
wherein (IIb) represents an amide containing n-alkylene-nitrogen spacer that is connected at the CH₂-end to the core or to a BU that is closer to the core, and that is connected at the N-end to two terminal units (TUs) or to two branching units (BUs) that are closer to the TUs, where the connections are given in wavy bonds, wherein q is 1, 2, 3, 4 or 5, and wherein R₁ is a hydrogen, a methyl, an ethyl, an n-propyl or an iso-propyl group; and
wherein (IIc) and (I Id) are defined as (IIa) and (IIb), respectively, and wherein R₂ is a methyl, ethyl, n-propyl, benzyl, acetamide (—CH₂—C(O)NH₂) or 2-hydroxy-ethylene group, and wherein X⁻ is the counter anion to the quaternary amine cation moiety; and
wherein y represents the specific and discrete generation number of the polyvalent molecule, where this number indicates how many successive BU layers are incorporated in the polyvalent molecule, as counted from traveling from the inner core to the outer TU groups (e.g. from traveling from the core to the TU groups), and where y is 0 or 1 or 2 or 3 or 4 or 5; and
wherein for each separate generational layer the BU can independently be selected from formulas (IIa), (IIb), (IIc) or (IId), i.e. the first BU emanating from the core, representing the first generational layer BU, can be different from the second BU emanating further away from the core (representing the second generational layer BU), and this one may be different from the third, the fourth and the fifth BU emanating further away from the core; and
wherein the terminal units (TU) are individually and independently chosen from a hydrogen and a C₁-C₃₀ alkyl, aryl, arylene-alkyl or alkylene-aryl group that optionally contains 1 to 8 heteroatoms that are individually and independently selected from the group consisting of O and N, with the proviso that not all TUs in the polyvalent molecule are hydrogens; and
wherein z represents the total number of TU groups that are attached to the polyvalent molecule, where z is 1 to 128; and
wherein the polyvalent molecule as according to formula (I) is polyvalent in positively ionizable and/or cationic groups, where the cumulative number of positively ionizable and cationic groups in the polyvalent molecule is 2 or higher.

As the branching units BU branch in two directions, z equals x*2^{y}. For example a second generation polyvalent molecule (y=2) with 4 connections from the core (x=4) will bear 4*2²=16 terminal units TU. In another example, a third generation polyvalent molecule (y=3) with 4 connections from the core (x=4) will bear 4*2³=32 terminal units TU. In another example, a zero generation polyvalent molecule (y=0) with 5 connections from the core (x=5) will bear 5*2⁰=5 terminal units TU.

The nanoparticles described herein have an outer layer comprising mainly stabiliser, phospholipid and sterol, and a core comprising polyvalent molecules and the cargo (also referred to herein as payload), which is a nucleic acid. The nanoparticles can be used to deliver the cargo to its intended destination, e.g. a cell, tissue or organ. Preferably the nucleic acid cargo is delivered intracellularly in the target cell, tissue or organ.

The present invention is based on the realization that a nanoparticle as described herein can successfully be loaded with nucleic acids and may be used to deliver said nucleic acids intracellularly, e.g. in the intended target cell. This was achieved by a combination of the following features:
the use of a polyvalent molecule to neutralize the nucleic acid to allow it to be loaded in the hydrophobic core of the nanoparticle;
defining the structural components of the nanoparticle and the ranges of their relative amounts, e.g. the amount of apolipoprotein, sterol, phospholipid, polyvalent molecule, nucleic acid and optionally filler molecules (e.g. triglycerides).

Additionally, the inventors have developed a method for successfully incorporating a nucleic acid in a stabiliser-based nanoparticle, preferably an apolipoprotein (and/or apolipoprotein mimetic)-based nanoparticle, as the individual components cannot simply be mixed to obtain nanoparticles as described herein. It was found to be essential that a two-step formulation process is performed, where in the first step a nucleic acid containing nanoparticle is formed and in the next step the stabiliser (e.g. an apolipoprotein, an apolipoprotein derivative, an apolipoprotein mimetic and/or an apolipoprotein mimetic derivative) is included in the nanoparticle. Preferably, the first step is performed at low pH and the second step is performed at physiological pH. This finding allows for the first time to include nucleic acids in a nanoparticle as described herein, thus allowing delivery of said nucleic acids to a target cell.

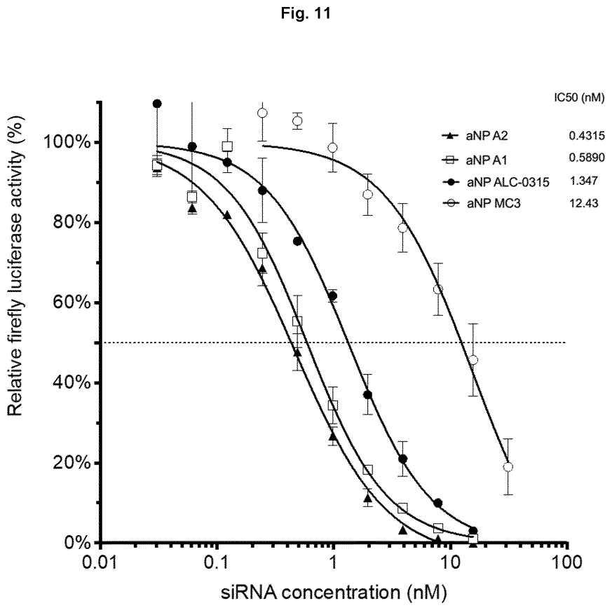

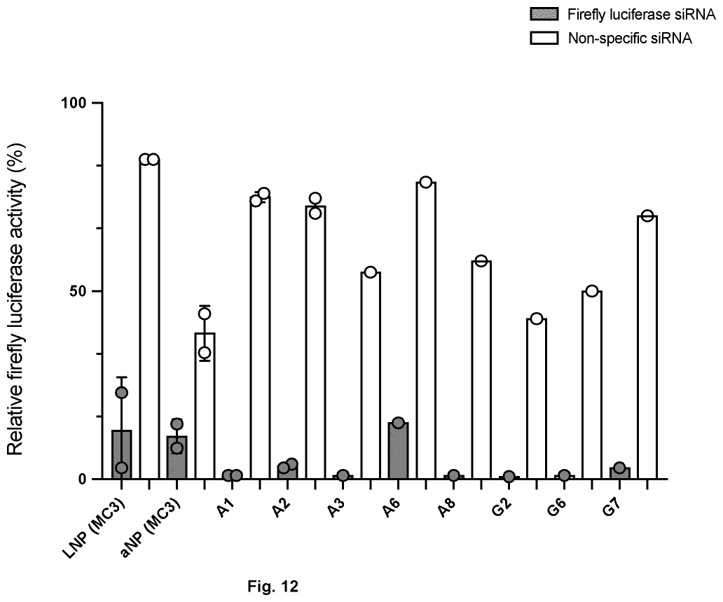

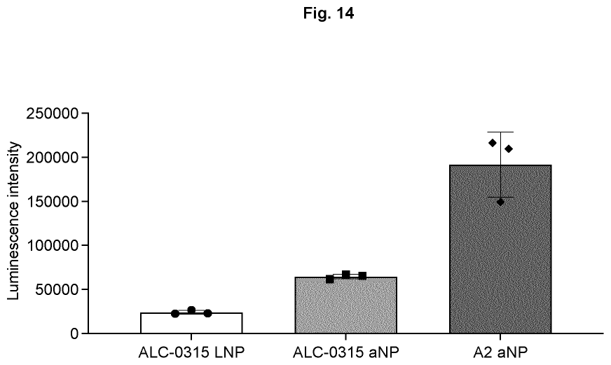

The present invention therefore provides polyvalent molecules that allow the loading of nucleic acids in a nanoparticle as described herein, meaning a nanoparticle with a hydrophobic core. The use of hydrophobic core-based nanoparticles has been described before in Jayaraman M et al. (Maximizing the potency of siRNA lipid nanoparticles for hepatic gene silencing in vivo. Angew Chem Int Ed Engl. 2012 Aug. 20; 51(34):8529-33) where nucleic acids are loaded in the core using positively charged amphiphiles such as Dlin-MC3-DMA. Such molecules typically comprise an amine group which can be protonated at lower pH. The positively charged amphiphilic molecule is then used to “neutralize” the negatively charged nucleic acid to allow loading in the hydrophobic core. Although this method has its merits, there are some concerns about the possible toxicity of the applied amphiphiles. Therefore, alternative solutions are desired. The present inventors herein describe the finding that the polyvalent molecules as defined herein may be used for this purpose. Furthermore, and importantly, the present inventors surprisingly found that using these polyvalent molecules, nucleic acids can be more efficiently delivered to a target cell to exert their function. For example FIGS. 11 and 12 describe how several formulations according to the present invention were more efficient in silencing a luciferase reporter gene by delivering specific siRNA to the cell, as aNP or LNP control formulations containing the ionizable cationic lipid DLin-MC3-DMA. In addition, FIG. 14 describes how a formulation according to the present invention is more efficient in expressing a luciferase reporter gene by delivering specific mRNA to the cell, as compared to aNP or LNP control formulations containing the ionizable cationic lipid ALC-0315. FIG. 16 describes efficient delivery of a formulation according to the present invention to spleen and bone marrow tissue in mice.

a. The Polyvalent Molecules

The polyvalent molecules as according to formula (I) are molecules with a dendrimer(-like) branched architecture, and are composed of three units: the core unit, the branching units (BUs) and the terminal units (TUs). Optionally, the BUs can be omitted.

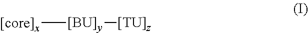

Particularly, from the inner core-unit the polyvalent molecules branch in an evenly fashion to the outer terminal units (TUs). Traveling from the core, every branching unit (BU) in the molecule splits in two directions, where both these directions are connected to either the next BU or to the TU.

Moreover, the polyvalent molecules described herein are defined as having specific and discrete generations. A polyvalent molecule of generation zero (G0, y=0) is a molecule without BUs wherein the core is directly attached to the TUs. A polyvalent molecule of the first generation (G1, y=1) is defined as a molecule with a core connected to 1 layer of BUs that are connected to the TUs; a polyvalent molecule of the second generation (G2, y=2) is defined as a molecule with a core connected to a first layer of BUs that is connected to a second layer of BUs, where these outer second layer BUs are connected to the TUs; a polyvalent molecule of the third generation (G3, y=3) is defined as a molecule with a core connected to a first layer of BUs that is connected to a second layer of BUs that is connected to a third layer of BUs, where these outer third layer BUs are connected to the Tus, and likewise for G4 (y=4) and G5 (y=5). Accordingly, the generation number of the polyvalent molecule—as represented by the letter y—indicates how many successive BU layers are incorporated in the polyvalent molecule, as counted from traveling from the inner core to the outer TU groups.

For illustration and clarification the below Scheme 1 shows non-limiting schematic examples of polyvalent molecules:
a generation 0 molecule (G0 material, i.e. y=0), with a 5-functional core (x=5) and 5 TU groups (z=5); and
a generation 1 molecule (G1 material, i.e. y=1), with a 4-functional core (x=4) and 8 TU groups (z=8); and
a generation 2 molecule (G2 material, i.e. y=2), with a 3-functional core (x=3) and 12 TU groups (z=12); and
a generation 3 molecule (G3 material, i.e. y=3), with a 2-functional core (x=2) and 16 TU groups (z=16),
wherein, x, y and z refer to the x, y and z in Formula (I). In Scheme 1, the branching units BU of every generational layer are indicated with its number.

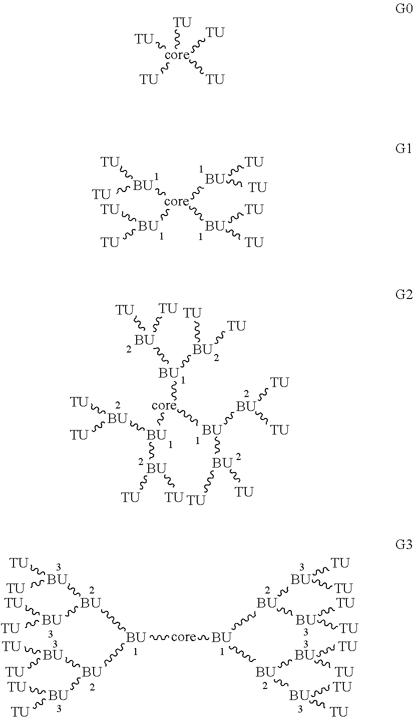

Scheme 1: Examples of Architectures of Dendritic Polyvalent Molecules of the Invention. G0, G1, G2 and G3 Molecules are Shown. For Clarity, the BU Layer of the Second Generation is Shown in Bold (for the G2 and G3 Molecules), as Opposed to the BUs of the First or Third Generation Layers.

The polyvalent molecules can be of generation 0 or 1 or 2 or 3 or 4 or 5 (y=0, 1, 2, 3, 4 or 5). In particular embodiments, the polyvalent molecule is of generation 0 (y=0). In an embodiment of the invention, the polyvalent molecule is of generation 1 (y=1). In an embodiment of the invention, the polyvalent molecule is of generation 2 (y=2). In an embodiment of the invention, the polyvalent molecule is of generation 3 (y=3). In an embodiment of the invention, the polyvalent molecule is of generation 4 (y=4). In an embodiment of the invention, the polyvalent molecule is of generation 5 (y=5).

In an embodiment of the invention, the polyvalent molecule is of a lower generation, where y=0 or y=1, more preferably y=1.

In an embodiment of the invention, the polyvalent molecule is of a higher generation, where y=2, 3, 4 or 5, preferably y=2 or y=3, more preferably y=2.

### Positively Ionizable Groups and Cationic Groups

The polyvalent molecule is polyvalent in positively ionizable and/or cationic groups, so that it can efficiently bind and capture nucleic acids.

A positively ionizable group is positively charged dependent of the pH of the surroundings: it can protonate at a sufficiently low pH to become positively charged. A positively ionizable group can be any kind of amine group. It is noted that amide, ester, ether, carbonate, urethane and urea groups are not positively ionizable groups. Particularly, positively ionizable groups are selected from tertiary amine, secondary amine, primary amine, guanidine and imidazole groups (Scheme 2). Preferred herein are tertiary amine, primary amine, guanidine and imidazole groups. More preferred are tertiary amine and guanidine groups. Most preferred are tertiary amine groups.

A cationic group is positively charged independent of the pH of the surroundings: it cannot deprotonate (to lose) or protonate (to gain) a positive charge. The cationic group can be any kind of cationic group. Preferably, cationic groups contain a nitrogen atom or multiple (up to 3, such as 2 or 3) nitrogen atoms. Cationic groups are preferably selected from quaternary ammonium, imidazolium and guanidinium groups (Scheme 2). Preferred cationic groups are quaternary ammonium and imidazolium groups. More preferred are quaternary ammonium groups.

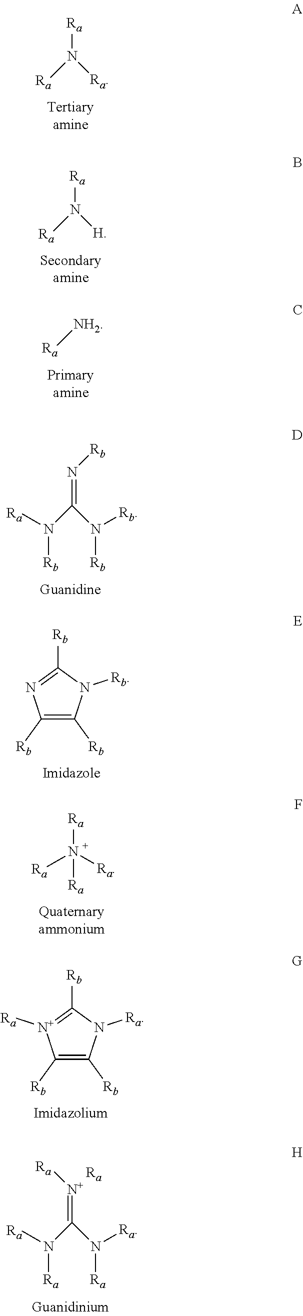

Scheme 2: Positively Ionizable Groups A to E (Also Referred to Herein as Formula's A to E) and Cationic Groups F to H (Also Referred to Herein as Formula's F to H). Rₐ can be any group (Alkyl, alkylene, aryl, arylene, alkylenearyl, alkylenearylene, arylenealkyl or arylenealkylene, optionally containing O or N heteroatoms), and it is not a hydrogen. R_{b} is similarly defined, but it can also be a hydrogen (for example, in formula E, not all 4 R_{b} Groups Need to be Hydrogen). The First Atoms Immediately Attached to Groups A to H are Carbons or Hydrogens and are not Heteroatoms.

The cumulative number of positively ionizable and/or cationic groups in a polyvalent molecule is at least 2. Preferably, the cumulative number of positively ionizable and/or cationic groups in a polyvalent molecule is is 3 or higher, 4 or higher, 5 or higher, 6 or higher, 8 or higher, 10 or higher, 12 or higher, or 14 or higher. Preferably, the cumulative number of positively ionizable and/or cationic groups in a polyvalent molecule is a discrete number for a polyvalent molecule, and is not an average value.

Herein, preferably only the positively ionizable and/or cationic groups are counted that are spaced by at least 3 atoms—preferably by carbon atoms—with respect to its neighbouring positively ionizable and/or cationic groups. For example, the two amines in a piperazine ring are then not counted.

In an embodiment all positively ionizable groups and/or cationic groups within the polyvalent molecule are spaced by at least 3 atoms from each other, where these spacer atoms are carbon, nitrogen and/or oxygen atoms.

In an embodiment of the invention, the polyvalent molecule only has positively ionizable groups and no cationic groups. Preferably, the positively ionizable charges then reside in the core and in BUs, and not in TUs. Alternatively, the positively ionizable charges reside in the core, in BUs and in TUs. In another option, the positively ionizable charges reside in the core and in TUs, and not in BUs. Finally, in particular embodiments, the positively ionizable charges reside in the core only. In this embodiment of the invention the positively ionizable groups are preferably tertiary amines, optionally combined with primary amines and/or guanidines, more preferably the positively ionizable groups are tertiary amines.

In an embodiment of the invention, the polyvalent molecule has a combination of positively ionizable groups and cationic groups. Preferably, the cationic charges then reside in the core only, and not in BUs or TUs. Alternatively, the cationic charges then reside in the core, and in the first generational BU-layer, and not in further BUs, nor in TUs. In these ways, a polyvalent molecule is generated that combines shielded cationic groups with outer positively ionizable groups. In this embodiment the cationic groups are preferably quaternary ammonium groups; and the positively ionizable groups are preferably tertiary amines, optionally combined with guanidines, more preferably these groups are tertiary amines.

In an embodiment of the invention, the polyvalent molecule only has cationic groups and no positively ionizable groups. Preferably, the cationic charges then reside in the core, and not in BUs nor in TUs. Alternatively, the cationic charges then reside in the core and in the first generational BU layer, and not in further BUs, nor in TUs. In this embodiment the cationic groups are preferably quaternary ammonium groups, optionally combined with guanidiniums, more preferably these groups are quaternary ammonium groups only.

The Multifunctional Core (i.e. The Center of the Polyvalent Molecule)

The polyvalent molecule has formula (I): [core]ₓ-[BU]_{y}-[TU]_{z}

In formula (I) the core is a nitrogen, or a C₁-C₁₈ linear, branched or cyclic group that contains 1 to 15 nitrogen heteroatoms and that optionally contains 1 to 4 oxygen heteroatoms.

The core of the polyvalent molecule contains 1 to 15 nitrogen atoms, preferably 2 to 10 nitrogen atoms, more preferably 2 to 4 nitrogen atoms, such as 2, 3 or 4 nitrogen atoms. These nitrogen atoms may be part of an amide, urethane or urea group, or they may be part of a positively ionizable group or a cationic group within the polyvalent molecule. Preferably, at least 2 nitrogen atoms in the core are part of a positively ionizable group or a cationic group within the polyvalent molecule. Preferably, all nitrogen atoms in the core are part of a positively ionizable group or a cationic group within the polyvalent molecule, or they are part of an amide group. More preferably, all nitrogen atoms in the core are part of a positively ionizable group or a cationic group within the polyvalent molecule.

The nitrogen atoms in the core that are part of a positively ionizable group or a cationic group within the polyvalent molecule are preferably spaced by at least 3 atoms from each other, where these spacer atoms are carbon, nitrogen and/or oxygen atoms, preferably carbon atoms only.

The core may contain functional groups such as amide, urethane, urea or ether groups. From these functional groups, amides and ethers are preferred. Amides are more preferred. Most preferably, however, the core does not contain any of these functional groups.

The core may contain 0 (in case the core is a nitrogen) to 18 carbon atoms, preferably 2 to 12 carbon atoms, and more preferably 4 to 10 carbon atoms.

The core may contain 0 to 4 oxygen atoms, preferably 0 to 2 oxygen atoms, and more preferably the core does not have any oxygen atoms.

The core may be a linear, branched or cyclic group, and is preferably linear or branched, more preferably linear.

The core has x number of connections from the core to the first generational branching units BU (or to the terminal units TU, when y=0), where x is 1, 2, 3, 4, 5, 6, 7, 8, 9, 10 or 12 and where these connections are all made from nitrogen atoms of the core to carbon atoms of the branching unit BU (or of the terminal unit TU).

Preferably, x is 2 or higher, more preferably x is 3 or higher, even more preferably x is 4 or higher. Preferably, x is a discrete number, and not an averaged number so as to account for multiple cores. Preferably, the core is a single entity (i.e. single chemical group), and not a mixture of entities. For example, the core is derived from n-butylene-diamine and not from both n-butyleen-diamine and n-propylene-diamine.

In an embodiment of the invention, nitrogen atoms of the core units are part of a positively ionizable group in the polyvalent molecule.

The below Schemes 3 and 4 show preferred core structures. The wavy bonds indicate the connections to the BUs of the first generational layer, or to the TUs (in case y=0). Accordingly, the number of wavy bonds for a specific core unit represents the value for x for that core.

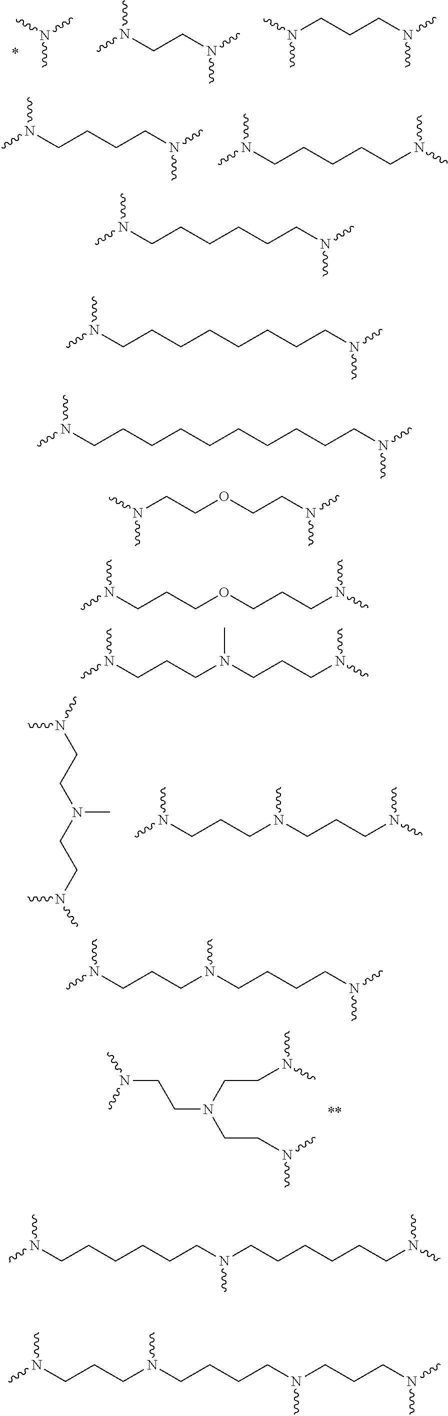

Scheme 3: Example Core Structures, with x=3 (Top Left), x=4 (First Three Rows), x=5 (Lower Two Rows; Left and Middle), and x=6 (Lower Two Rows; Right). All these Cores are Linear, Apart from Those Indicated with * (Nitrogen Core) or with ** (Branched Core).

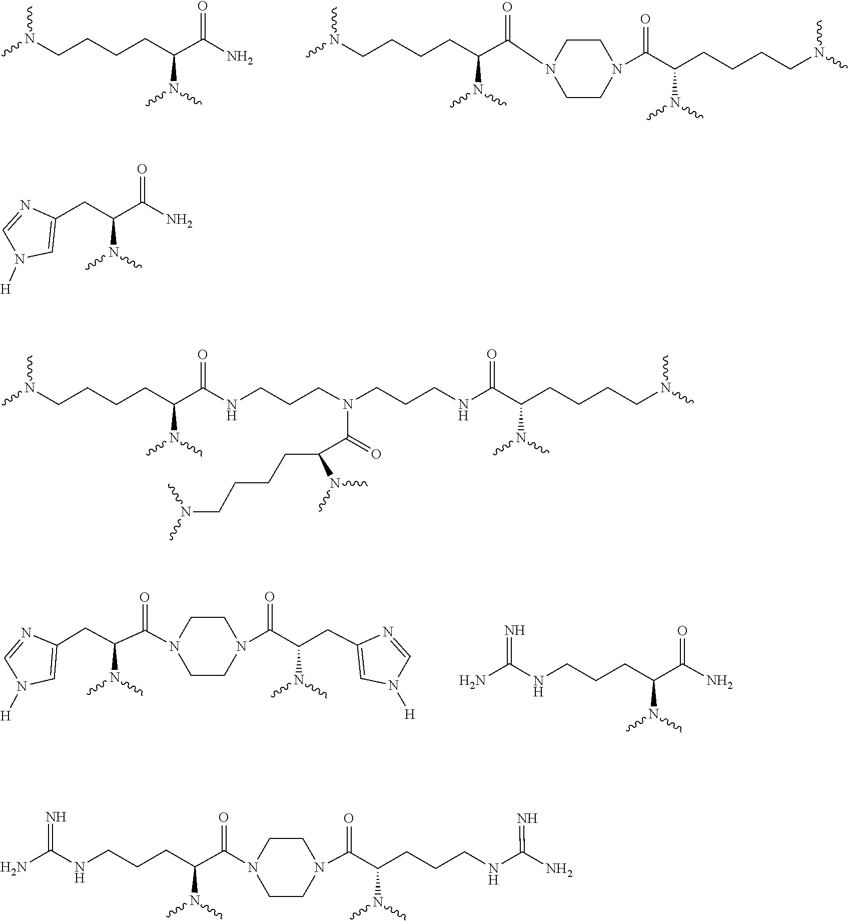

Scheme 4: Non-Limiting Example Core Structures as Derived from Lysine, Histidine or Arginine Amino Acid Building Blocks, with x=2, 4, 8 or 12.

The core units can be seen as derived from amine functional molecules: replace all x connections of the core to BUs (or to TUs) with hydrogen atoms, and then these amine functional molecules become apparent. Preferably, the core units are derived from single compound amines that are pure, i.e. with a purity of about 90% or higher, preferably about 97% or higher, more preferably about 98% or higher, even more preferably about 99% or higher.

Accordingly, core units are preferably derived from ammonia, 1,2-diamino-ethane, 1,3-diamino-propane, 1,4-diamino-butane (or putrescine), 1,5-diamino-pentane, 1,6-diamino-hexane, 1,8-diamino-octane, 1,10-diamino-decane, 2-(2-aminoethoxy)ethylamine, 3-(3-aminopropoxy)propylamine, 3,3′-diamino-N-methyldipropylamine, N-(3-aminopropyl)-1,3-propanediamine (or norspermidine), N-(4-aminobutyl)-1,4-butanediamine, N-(6-aminohexyl)-1,6-hexanediamine, spermine, spermidine or N,N-bis(2-aminoethyl)-1,2-ethanediamine. (Note that 3-(3-aminopropoxy)propylamine and 3,3′-diamino-N-methyldipropylamine can alternatively be viewed as an oxygen or an methyl-nitrogen core, respectively, with two attached n-propylene-nitrogen BUs). From this group, core units are more preferably derived from ammonia, 1,4-diamino-butane (or putrescine), 1,6-diamino-hexane, 3,3′-diamino-N-methyldipropylamine, N-(3-aminopropyl)-1,3-propanediamine (or norspermidine), N-(4-aminobutyl)-1,4-butanediamine, spermine or spermidine. Even more preferred herein are ammonia, 1,4-diamino-butane (or putrescine) or N-(3-aminopropyl)-1,3-propanediamine (or norspermidine).

Furthermore, core units can be derived from amino acids such as lysine, arginine or histidine, preferably in their naturally occurring L-stereomeric forms. Non-limiting examples are core units derived from dimers or trimers of these amino acids, or cores derived from C-amidated amino acids (using ammonia, or primary or secondary amines), see Scheme 4 for several examples.

In an embodiment of the invention, the core units are derived from naturally occurring amine functional molecules, such as ammonia, 1,4-diamino-butane (or putrescine), N-(3-aminopropyl)-1,3-propanediamine (or norspermidine), spermine, L-lysinamide or L-argininamide. Preferred herein are ammonia, 1,4-diamino-butane (or putrescine), N-(3-aminopropyl)-1,3-propanediamine (or norspermidine) or spermine. More preferred are 1,4-diamino-butane (or putrescine) or N-(3-aminopropyl)-1,3-propanediamine (or norspermidine).

In an embodiment of the invention, nitrogen atoms of the core units are part of cationic groups in the polyvalent molecule. Preferably, these nitrogen atoms are then part of quaternary ammonium groups. The below Scheme 5 shows preferred core structures for this embodiment. The wavy bonds indicate the connections to the BUs of the first generational layer, or to the TUs (in case y=0). Accordingly, the number of wavy bonds for a specific core unit represents the value for x for that core. The R₃ group is a methyl, ethyl, n-propyl, benzyl, acetamide (—CH₂—C(O)NH₂) or 2-hydroxy-ethylene group, preferably a methyl, ethyl, benzyl or acetamide group, more preferably a methyl or ethyl group. For convenience the counter anions X⁻ that neutralize the cations have not been drawn in Scheme 5. This X⁻ counter anion can be any anion, also a doubly or triply charge anion. Preferably, X⁻ is a chloride Cl⁻, a bromide Br⁻, an iodide I⁻, a tosylate TsO⁻, a carbonate, an oxalate, a sulfate or a phosphate anion. More preferably, X⁻ is a chloride, an oxalate, a phosphate, or a bromide, most preferably a chloride or a phosphate (including monohydrogen- or dihydrogen phosphates).

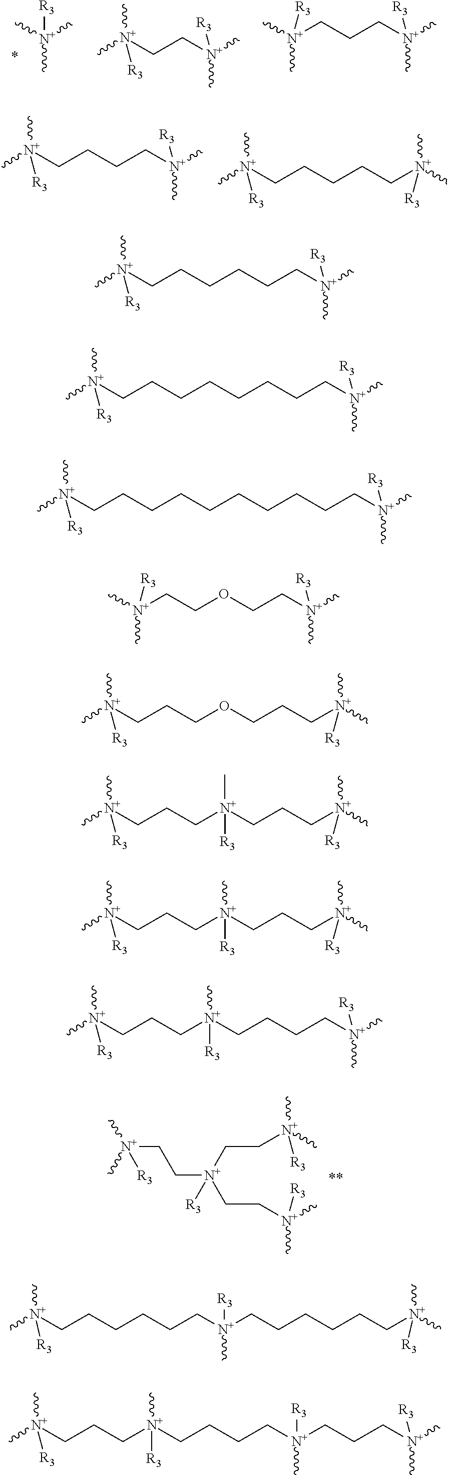

Scheme 5: Examples of Core Structures with Cationic Groups that are Part of the Polyvalent Molecule. For Convenience the Counter Anions X— that Neutralize the Cations are not Shown in the Structures Shown in Scheme 5.

### The Branching Units BU

The branching unit BU has the formula (IIa), (IIb), (IIc) or (IId):

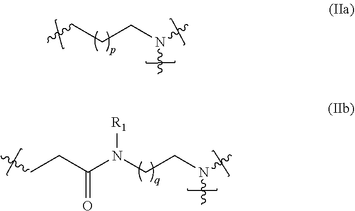

Formula (IIa) represents an n-alkylene-nitrogen spacer that is connected at the CH₂-end to the core or to a BU that is closer to the core, and that is connected at the N-end to two terminal units (TUs) or to two branching units (BUs) that are closer to the TUs, where the connections are given in wavy bonds, and wherein p is 1, 2, 3 or 4.

Preferably, in (IIa) p is 1 or 2 or 3, preferably 1 or 2, so that (IIa) represents an n-propylene-nitrogen spacer, an n-butylene-nitrogen or an n-pentylene-nitrogen spacer, preferably n-propylene-nitrogen spacer, or an n-butylene-nitrogen. More preferably, p is 1, and (IIa) represents an n-propylene-nitrogen spacer.

Formula (IIb) represents an amide containing n-alkylene-nitrogen spacer that is connected at the CH₂-end to the core or to a BU that is closer to the core, and that is connected at the N-end to two terminal units (TUs) or to two branching units (BUs) that are closer to the TUs, where the connections are given in wavy bonds, wherein q is 1, 2, 3, 4 or 5, and wherein R₁ is a hydrogen, a methyl, an ethyl, an n-propyl or an iso-propyl group.

Preferably, in (IIb) q is 1, 2 or 3, more preferably 1 or 2, most preferably 1.

Preferably, in (IIb) R₁ is a hydrogen or a methyl, more preferably R₁ is a hydrogen.

Preferably, in (IIb) q is 1 and R₁ is a hydrogen, q is 1 and R₁ is a methyl, q is 2 and R₁ is a hydrogen, q is 2 and R₁ is a methyl, or q is 3 and R₁ is a hydrogen. More preferably, in (IIb) q is 1 and R₁ is a hydrogen, q is 2 and R₁ is a hydrogen, or q is 2 and R₁ is a methyl. Even more preferred, in (IIb) q is 1 and R₁ is a hydrogen.

Formula (IIc) is defined as above for (IIa), and moreover R₂ is a methyl, ethyl, n-propyl, benzyl, acetamide (—CH₂—C(O)NH₂) or 2-hydroxy-ethylene group, and X⁻ is the counter anion to the quaternary amine cation moiety.

Preferably, in (IIc) R₂ is a methyl, an ethyl or a benzyl, more preferably a methyl.

In (IIc), the X⁻ counter anion can be any anion. Preferably, X⁻ is a chloride Cl⁻, a bromide Br⁻, an iodide I⁻, a tosylate TsO⁻ or a dihydrogen-phosphate. More preferably, X⁻ is a chloride or a dihydrogen-phosphate, most preferably a chloride.

In (IIc), the X⁻ counter anion can also be a doubly or triply charges anion, such as a carbonate, an oxalate, a sulfate, a monohydrogen-phosphate or a phosphate anion, more preferably an oxalate or a monohydrogen-phosphate HPO₄²⁻ anion. In these cases, the counter anion neutralizes multiple branching units (IIc).

Preferably, in (IIc) R₂ is a methyl and X⁻ is a chloride, or R₂ is a benzyl and X⁻ is a chloride.

Preferably, in (IIc) p is 1, R₂ is a methyl and X⁻ is a chloride, p is 1, R₂ is a benzyl and X⁻ is a chloride, p is 2, R₂ is a methyl and X⁻ is a chloride, or p is 2, R₂ is a benzyl and X⁻ is a chloride. More preferably, p is 1, R₂ is a methyl and X⁻ is a chloride, or p is 1, R₂ is a benzyl and X⁻ is a chloride.

Formula (IId) is defined as above for (IIb), and moreover R₂ is a methyl, ethyl, n-propyl, benzyl, acetamide (—CH₂—C(O)NH₂) or 2-hydroxy-ethylene group, and X⁻ is the counter anion to the quaternary amine cation moiety.

Preferably, in (IId) R₂ is a methyl, an ethyl or a benzyl, more preferably a methyl.

In (IId), the X⁻ counter anion can be any anion. Preferably, X⁻ is a chloride Cl⁻, a bromide Br⁻, an iodide I⁻, a tosylate TsO⁻ or a dihydrogen-phosphate. More preferably, X⁻ is a chloride or a dihydrogen-phosphate, most preferably a chloride.

In (IId), the X⁻ counter anion can also be a doubly or triply charges anion, such as a carbonate, an oxalate, a sulfate, a monohydrogen-phosphate or a phosphate anion, more preferably an oxalate or a monohydrogen-phosphate HPO₄²⁻ anion. In these cases, the counter anion neutralizes multiple branching units (IId).

Preferably, in (IId) R₂ is a methyl and X⁻ is a chloride, or R₂ is a benzyl and X⁻ is a chloride.

From the branching units as according to formulas (II-a), (II-b), (II-c) and (II-d), the BUs of formulas (II-a), (II-b) and (II-c) are preferred. More preferred are BUs of formulas (II-a) and (II-b), and most preferred are BUs of formula (II-a).

Within the polyvalent molecule, the BU can independently be selected from formulas (IIa), (IIb), (IIc) or (IId) for each separate generational layer, i.e. the first BU emanating from the core, representing the first generational layer BU, can be different from the second BU emanating further away from the core (representing the second generational layer BU), and this one may be different from the third, the fourth and the fifth BU emanating further away from the core.

In an embodiment of the invention, the BUs of the polyvalent molecule are the same for each generational layer. For example, all BUs in a polyvalent molecule are of the formula (IIa), for example with p=1. In another example, all BUs in a polyvalent molecule are of the formula (IIa), with p=2. In another example, all BUs in a polyvalent molecule are of the formula (IIa), with p=3. In another example, all BUs in a polyvalent molecule are of the formula (IIb), with for example q=1 and R₁═H.

In an embodiment of the invention, BUs of formula (IIa) and (IIc) are combined within a polyvalent molecule. For example, BUs of the first generational layer are according to formula (IIc), while BUs of the other generational layer(s) are of type (IIa). In this example, the polyvalent molecule has shielded inner cationic groups and more exposed outer positively ionizable groups.

In an embodiment of the invention, BUs of formula (IIb) and (IId) are combined within a polyvalent molecule. For example BUs of the first generational layer are according to formula (IId), while BUs of the other generational layer(s) are of type (IIb). In this example the polyvalent molecule has shielded inner cationic groups and more exposed outer positively ionizable groups.

In an embodiment of the invention, the polyvalent molecule does not have any branching groups BU, and y is then 0.

### The Terminal Units TU

In formula (I), the terminal units (TU) are individually and independently chosen from a hydrogen and a C₁-C₃₀ alkyl, aryl, arylene-alkyl or alkylene-aryl group that optionally contains 1 to 8 heteroatoms that are individually and independently selected from the group consisting of O and N, with the proviso that not all TUs in the polyvalent molecule are hydrogens.

A terminal unit may be devoid of functional groups, i.e. consisting only of carbon(s) and hydrogen(s). Alternatively, a terminal unit may contain one or more functional groups, preferably selected from amide, urethane, urea, ester, hydroxy and ether groups. From these functional groups, amides, esters and hydroxy groups are preferred.

In formula (I), the TUs can individually and independently be selected from any of the following groups (Scheme 6), as according to the formulas (III-a) to (III-j). In every of these formulas the wavy bond indicates the connection to a nitrogen atom of an outer BU or of the core (in case y=0).

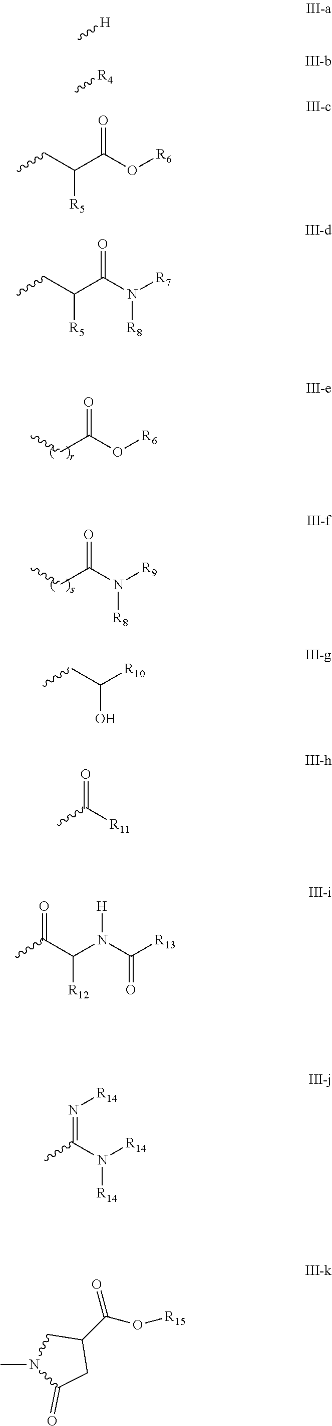

Scheme 6: Formulas (III-a) to (III-k) Representing Possibilities for the TU Group. The Wavy Bonds are Connections to a BU, Preferably an Outer BU, or to the Core.

In formula (III-a) the TU is a hydrogen.

In formula (III-b), the R₄ group preferably is a linear or branched C₁-C₁₈ alkyl group or a (substituted) benzyl group, optionally containing heteroatoms independently selected from O and N. More preferably R₄ is a C₁-C₁₂ linear or branched alkyl group or a benzyl group. The alkyl group can be saturated or unsaturated, and is preferably saturated. In some embodiments the alkyl group is saturated or has one, two or three unsaturations. Optionally, R₄ comprises a positively ionizable group or a cationic group, such as a tertiary amine or a quaternary ammonium group.

In formula (III-c), R₅ preferably is a hydrogen or a methyl group, more preferably a hydrogen. In particular embodiments, such as in case R₅ is a methyl group, then the stereo-chemistry in (III-c) is not defined. The R₆ group preferably is a linear or branched C₁-C₁₈ alkyl group, optionally containing heteroatoms independently selected from O and N; preferably R₆ is a linear or branched saturated or unsaturated C₁-C₁₇ alkyl group, optionally containing heteroatoms independently selected from O and N. More preferably, R₆ is a linear or branched C₂-C₁₄ alkyl group, even more preferably a linear or branched C₄-C₁₂ alkyl group. The alkyl group can be saturated or unsaturated, and is preferably saturated. In some embodiments the alkyl group is saturated or has one, two or three unsaturations.

In formula (III-d), R₅ preferably is a hydrogen or a methyl group, more preferably a hydrogen. In case R₅ is a methyl group, then the stereo-chemistry in (III-d) is not defined. The R₇ group preferably is a linear or branched C₁-C₁₈ alkyl group, optionally containing heteroatoms independently selected from O and N. More preferably, R₇ is a linear or branched C₂-C₁₄ alkyl group, even more preferably a linear or branched C₄-C₁₂ alkyl group. The R₈ group preferably is a hydrogen or a linear or branched C₁-C₁₈ alkyl group, optionally containing heteroatoms independently selected from O and N. In some embodiments R₈ is a hydrogen or a linear or branched C₁-C₁₂ alkyl group, optionally containing heteroatoms independently selected from O and N. Preferably, R₈ is a hydrogen or a linear or branched C₁-C₁₆ alkyl group, optionally containing oxygen heteroatoms; preferably, R₈ is a hydrogen or a linear or branched C₁-C₁₄ alkyl group, optionally containing oxygen heteroatoms; preferably, R₈ is a hydrogen or a linear or branched C₁-C₁₀ alkyl group, optionally containing oxygen heteroatoms. More preferably, R₈ is a hydrogen or a linear or branched C₁-C₁₂ alkyl group, optionally containing oxygen heteroatoms. Even more preferably R₈ is a hydrogen, a methyl, a 2-hydroxyethyl, a 3-hydroxypropyl or a C₂-C₈ alkyl group. The alkyl group in R₇ or R₈ can be saturated or unsaturated, and is preferably saturated.

In formula (III-e), r=0 or 1, preferably 1, or a number selected from 3 to 10; r is preferably 1, 5 or 10; preferably r is 0, 1, 5 or 10. In formula (III-e), the R₆ group preferably is a linear or branched C₁-C₁₈ alkyl group, optionally containing heteroatoms independently selected from O and N. More preferably, R₆ is a linear or branched C₂-C₁₄ alkyl group, even more preferably a linear or branched C₄-C₁₂ alkyl group. The alkyl group can be saturated or unsaturated, and is preferably saturated. In some embodiments the alkyl group is saturated or has one, two or three unsaturations.

In formula (III-f), s=0 or 1, preferably 1, or a number selected from 3 to 10; s is preferably 1, 5 or 10. The R₈ group preferably is a hydrogen or a linear or branched C₁-C₁₈ alkyl group, optionally containing heteroatoms independently selected from O and N. Preferably R₁₀ is a C₁-C₁₆ linear or branched alkyl group; preferably R₁₀ is a C₁-C₁₄ linear or branched alkyl group. More preferably, R₈ is a hydrogen or a linear or branched C₁-C₁₂ alkyl group, optionally containing oxygen heteroatoms. Even more preferably R₃ is a hydrogen, a methyl, a 2-hydroxyethyl, a 3-hydroxypropyl or a C₂—C alkyl group. The R₉ group preferably is a hydrogen or a linear or branched C₁-C₁₈ alkyl group, optionally containing heteroatoms independently selected from O and N. More preferably, R₉ is a hydrogen or a linear or branched C₂-C₁₄ alkyl group, even more preferably a hydrogen or a linear or branched C₄-C₁₂ alkyl group. The alkyl group in R₃ or R₉ can be saturated or unsaturated, and is preferably saturated.

In formula (III-g), the R₁₀ group preferably is a linear or branched C₁-C₁₈ alkyl group, optionally containing heteroatoms independently selected from O and N. More preferably R₁₀ is a C₄-C₁₆ linear or branched alkyl group. The alkyl group can be saturated or unsaturated, and is preferably saturated. In particular embodiments, the stereo-chemistry in (III-g) is not defined, as the hydroxy group is connected in an undefined fashion (racemic). Optionally, the hydroxy group in (III-g) is acylated creating an ester group.

In formula (III-h), the R₁₁ group preferably is a linear or branched C₁-C₂₉ alkyl group, optionally containing heteroatoms independently selected from O and N. Preferably R₁₁ is a linear or branched C₁-C₂₇ alkyl group, preferably R₁₁ is a linear or branched C₁-C₂₅ alkyl group; preferably R₁₁ is a linear or branched C₁-C₁₇ alkyl group, optionally containing heteroatoms independently selected from O and N. Preferably R₁₁ is a C₁-C₁₅ linear or branched alkyl group; preferably R₁₁ is a C₁-C₁₃ linear or branched alkyl group, each of said groups containing heteroatoms independently selected from O and N. More preferably R₁₁ is a C₁-C₁₁ linear or branched alkyl group. The alkyl group can be saturated or unsaturated, and is preferably saturated. In some embodiments the alkyl group is saturated or has one, two or three unsaturations. Optionally, R₁₁ comprises a positively ionizable group or a cationic group, such as a tertiary amine, guanidine or quaternary ammonium group, preferably a tertiary amine or guanidine group.

In formula (III-i), the R₁₃ group preferably is a linear or branched C₁-C₁₇ alkyl group, optionally containing heteroatoms independently selected from O and N. More preferably R₁₃ is a C₁-C₁₇ linear or branched alkyl group. The alkyl group can be saturated or unsaturated, and is preferably unsaturated. The R₁₂ group preferably is the residue of an amino acid, more preferably of a lysine, arginine or histidine (preferably in their naturally occurring L-stereomeric forms), or is a derivative of such a residue. Accordingly, R₁₂ may contain a primary amine, a guanidine, or an imidazole group, or any derivatizations of these groups; as a result these TUs contain a positively ionizable or a cationic group.

In formula (III-j) the terminal unit is an amidine group. Note, however, that this group—together with the nitrogen atom of the BU unit or core unit to which it is attached—forms a guanidine group. The R₁₄ group can be independently selected for every position, and preferably is selected from a hydrogen and a linear or branched C₁-C₁₈ alkyl group, optionally containing heteroatoms independently selected from O and N. More preferably, the R₁₄ group is selected from a hydrogen and a linear C₁-C₁₂ alkyl group. The alkyl group can be saturated or unsaturated, and is preferably saturated.

In formula (III-k), R₁₅ is a linear or branched C₁-C₁₈ alkyl group, optionally containing heteroatoms independently selected from O and N; preferably R₂ₐ is a linear or branched saturated or unsaturated C₁-C₁₅ alkyl group, optionally containing heteroatoms independently selected from O and N. More preferably, R₂ₐ is a linear or branched C₂-C₁₃ alkyl group, even more preferably a linear or branched C₂-C₁₁ alkyl group. The alkyl group can be saturated or unsaturated, and is preferably saturated. In some embodiments the alkyl group is saturated or has one, two or three unsaturations.

From the terminal units TU as according to formulas (III-a) to (III-k), TUs of formulas (III-a) to (III-j) are preferred and TUs of formulas (III-b), (III-c), (III-d), (III-f), (III-g) and (III-j) are more preferred. Even more preferred are TUs of formulas (III-b), (III-c), (III-d), (III-f) and (III-g), or of formulas (III-b), (III-c), (III-d) and (III-f). Even more preferred are TUs of formulas (III-c), (III-d), (III-f) and (III-g) or formulas (III-c), (III-d), and (III-f).

In an embodiment of the invention, all TUs of the polyvalent molecule are the same, or are of the same formula (III) type. Preferably, all TUs of the polyvalent molecule are the same. For example, all TUs in a polyvalent molecule are of formula (III-d), for example with R₅ is a hydrogen, R₃ is a hydrogen and R₇ is an n-decyl group for all TUs. In another example, all TUs in a polyvalent molecule are of formula (III-c), for example with R₅ is a hydrogen and R₆ is an n-octyl group for all TUs. In another example, all TUs in a polyvalent molecule are of formula (III-g), for example with R₁₀ is an n-decyl group for all TUs, preferably with the hydroxy group connected in a random (racemic) fashion for all TUs.

In an embodiment of the invention, every nitrogen of the outer BU units is connected to two particular TUs as selected from the formulas (III). In particular embodiments, it is preferred that every nitrogen of the outer BU units is connected to two specific TUs as selected from the formulas (III). Accordingly, (about or exactly) 50% of the TUs is of the first particular selection TU-1 and (about or exactly 50%) is of the second particular selection TU-2. Suitable combinations for TU-1 and TU-2 are particular combinations of (III-a) and (III-h); (III-a) and (III-i); (III-a) and (III-j); (III-b) and (III-c); (III-b) and (III-d). For clarification, see Scheme 7 below with these examples. Other combination examples are those of (III-c) and (III-h); (III-d) and (III-h). Yet other combination examples are those of (III-j) with either (III-b), (III-d) or (III-f).

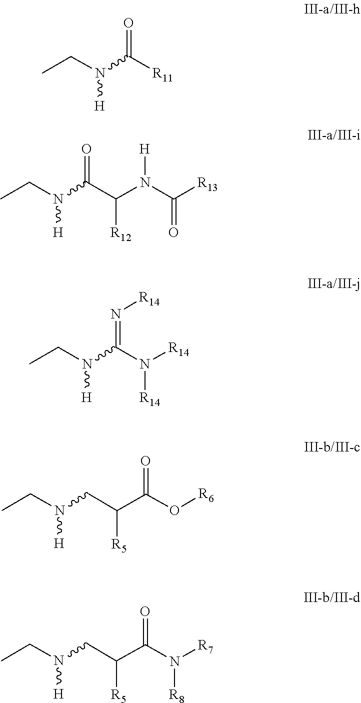

Scheme 7: Example of TU-1 and TU-2 Combinations as Substituted on Every Nitrogen of the Outer Branching Units (this Nitrogen is Shown in Bold). Specific Examples for these 5 Structures are: R₁₁ is a n-C9 Group for all End Groups; R₁₂ is a L-Lysine residue and R₁₃ is a n-C11 group for all end groups; all R₁₄ groups are n-Butyl groups for all end groups; R₄ is methyl, R₅ is hydrogen and R₆ is an n-Octyl group for all end groups; R₄ is methyl, R₅ is hydrogen, R₇ is an n-Octyl group and Rₑ is a Hydrogen for all End Groups.

In an embodiment of the invention, 2 or more TUs, preferably 2, 3 or 4 TUs, more preferably 2 TUs as selected from the formulas (III) are applied in the polyvalent molecule. In this embodiment TU-1 and TU-2 (or TU-1, TU-2 and TU-3; etc.) can be randomly distributed over the nitrogens of the outer BU units. TU-1 and TU-2 may be abundant in different ratios, ranging from about 1%-99% to about 99%-1%, preferably from about 10%-90% to about 90%-10%, more preferably from about 30%-70% to about 70%-30%. Suitable combinations for TU-1 and TU-2 are particular combinations of (III-b) and either (III-c), (III-d), (III-f), (III-g) or (III-j); or particular combinations of (III-h) and either (III-c) or (III-d). Preferably, in this embodiment, the chosen TUs are specific TUs: for example TU-1 is (III-b) with R₄ a methyl group and TU-2 is (III-d) with R₅ a hydrogen, R₇ an n-octyl group and R₈ a hydrogen.

In an embodiment of the invention a larger portion of the TUs are hydrogens. Relative to the total amount of TUs, 10% of the TUs or more are hydrogens, or 20% or more, 30% or more, 40% or more, 50% or more, 60% or more, or 70% or more.

In formula (I) z represents the total number of TU groups that are attached to the polyvalent molecule, where z is 1 to 128. Preferably z is 4 or higher, more preferably z is 6 or higher, even more preferably z is 8 or higher. In an embodiment of the invention, z is 10 or higher, 16 or higher, or even 20 or higher. Preferably, z is lower than 129 or lower than 128, more preferably lower than 80, even more preferably lower than 50.

The polyvalent molecule comprises multiple hydrophobic groups, mostly or exclusively residing in the TUs, so that this molecule can encapsulate the nucleic acid and thereby create a hydrophobic shell or cover around the assembly of the polyvalent molecule(s) and the nucleic acid(s).

Accordingly, at least a part (i.e. a certain percentage) of all the TUs in the polyvalent molecule are hydrophobic in nature, so as to render the polyvalent molecule hydrophobic or amphiphilic. The hydrophobic TUs preferably comprise a C₁-C₁₈ alkyl, aryl, arylene-alkyl or alkylene-aryl chain. More preferably, these Tus, preferably hydrophobic Tus, comprise a C₄-C₁₃ aliphatic alkyl chain, where this chain may be linear or branched, and where it may be saturated or unsaturated in nature. In formula (I), preferably at least 5% of the terminal groups TU are hydrophobic in nature, or at least 10%, or at least 20%, or at least 30%, or at least 50%, or at least 60%, or at least 70%, or at least 80%, or at least 90%. In an embodiment of the invention all—or virtually all (>97%)—of the TUs are hydrophobic in nature.

### Aspects of the Polyvalent Molecule

In an embodiment of the invention, the polyvalent molecule is a single compound, i.e. not a mixture of compounds. In other words, in an embodiment, all polyvalent molecules within the nanoparticle as taught herein are the same. The purity of the polyvalent molecule/compound is then 60% or higher, preferably 85% or higher, more preferably 90% or higher, most preferably 95% or higher, such as 99% or higher, for example 100%. This typically applies to G0, G1 and G2 materials (y=0, 1 or 2). For the higher generation G3, G4 and G5 materials (y=3, 4 or 5), the dispersity D (=Mw/Mn) of the polyvalent molecules is typically lower than about 1.2, preferably lower than 1.1, more preferably lower than 1.03. The dispersity D of the material can be assessed by methods that are known in the art, for example GPC measurements or MS-spectroscopy (see e.g. Hummelen et al., Chem. Eur. J. 1997, 3, 9, 1489-1493).

In an embodiment of the invention, the polyvalent molecule is a mixture of compounds. In other words, in an embodiment, the nanoparticle as taught herein comprises a mixture of different polyvalent molecules. This may be due to the presence of undefined stereo-centers in the polyvalent molecule. An example is the presence in the polyvalent molecule of branched alkyl chains that are of racemic origin. Another example is the use of epoxy-alkyl reactants that result in the formation of stereo-chemically undefined (racemic) hydroxy groups in the polyvalent molecule. Accordingly, in particular embodiments, the polyvalent molecules within the nanoparticle as taught herein may not be pure, but the connectivity of the atoms within all polyvalent molecules within the nanoparticle as taught herein is the same.

The polyvalent molecule may have a specific core and may be of a specific generation (y=0 or 1 or 2 or 3 or 4 or 5), so is the polyvalent molecule may have a structure with evenly distributed BUs. Preferably, every nitrogen end group has the same specific TU attached (or it has the same two specific TUs attached), as selected from (III-a) to (III-k) Accordingly, the polyvalent molecule of the invention preferably has an overall shape that is symmetric, preferably when disregarding the chiral centra that may be present in the molecule.

In particular embodiments, the polyvalent molecule of the invention has a molecular weight that is higher than 400 Dalton, preferably higher than 800 Dalton, more preferably higher than 1200 Dalton, even more preferably higher than 1500 Dalton. In particular embodiments, the polyvalent molecule of the invention has a molecular weight that is lower than 80 kDalton, preferably lower than 40 kDalton, more preferably lower than 20 kDalton, even more preferably lower than 12 kDalton.

The molecular weight of the polyvalent molecule of the invention (MW) divided by the total number of positively ionizable and/or cationic groups (#N) in the molecule (or in other words the ratio of the molecular weight of the polyvalent molecule of the invention over the total number of positively ionizable and/or cationic groups in the molecule) indicates the concentration of these groups in the molecule. MW/#N is preferably higher than 80, more preferably higher than 150, and most preferably higher than 200. MW/#N is preferably lower than 900, more preferably lower than 600, and most preferably lower than 450. These numbers indicate that the concentration of groups capable of binding nucleic acids in the polyvalent molecules of the invention may be quite high. For example, amphiphilic molecules previously used in the art have MW/#N values of 642 (for DLin-MC3-DMA, or MC3) and 766 (for ALC-0315).

The polyvalent molecules of the invention bind stronger to nucleic acids than monovalent amphiphilic molecules do, due to the polyvalent effect. Accordingly, the nucleic acids are better bound and retained within the nanoparticles of the invention.

In addition, the polyvalent molecules of the invention may also interact with the apolipoprotein stabiliser (i.e. an apolipoprotein, an apolipoprotein derivative, an apolipoprotein mimetic and/or an apolipoprotein mimetic derivative), as this protein has an overall negative charge (such as described in Sparks D L, Lund-Katz S, Phillips M C. The charge and structural stability of apolipoprotein A-I in discoidal and spherical recombinant high density lipoprotein particles. J Biol Chem. 1992 Dec. 25; 267(36):25839-47. PMID: 1464598). As a result, the apolipoprotein, apolipoprotein derivative, apolipoprotein mimetic and/or apolipoprotein mimetic derivative becomes better attached to the nanoparticle of the invention. In circulation, e.g. in the blood, release of the apolipoprotein component (e.g. an apolipoprotein, an apolipoprotein derivative, an apolipoprotein mimetic and/or an apolipoprotein mimetic derivative) from the nanoparticle may therefore become less likely.

Transfection of the nucleic acid into the targeted cells may very well be aided by the presence of the polyvalent molecules of the invention, as these can efficiently bind and interact with multiple phospholipids at a (myeloid) cell barrier, thus creating a gap for the nucleic acids to enter.

The polyvalent molecule of the invention can be processed from solutions. Accordingly, the polyvalent molecule is preferably soluble in solvents ranging in polarity. Therefore, the polyvalent molecule is preferably soluble in tricaprylin, in ethanol or in iso-propanol, more preferably in all three of tricaprylin, ethanol and iso-propanol. The solubility can be checked by stirring about 20 mg of the polyvalent molecule in 1 gram of tricaprylin, ethanol or iso-propanol, and assessing whether all material spontaneously dissolves to create a clear/transparent solution (with a concentration of about 2 w/w %). The test can be done at about 20 degrees centigrade (room temperature) or at about 37 degrees centigrade. Preferably, the polyvalent molecule of the invention is soluble at room temperature already.

In an embodiment of the invention, the polyvalent molecule comprises ester, amide and/or hydroxy groups. Such groups give options for a slow in-vivo degradation and render the polyvalent molecules (more) biocompatible. Preferred groups are amides and/or esters.

The polyvalent molecules of the invention preferably are non-toxic, or they have a limited toxicity, either on their own, or when bound to nucleic acids. Toxicity cell tests can be executed by methods that are known in the art, such as for example by cell viability MTT assays, or similar or comparable tests.

### The Synthesis of the Polyvalent Molecule

The polyvalent molecule can be prepared by using any suitable synthetic method or route. It can conveniently be prepared by taking a multifunctional amine building block and modifying it with a molecule that is reactive towards amines and that is hydrophobic in nature. Alternatively, two or more amine-reactive molecules can be applied, at least one of which is hydrophobic in nature.

Particularly useful building blocks are (primary) amine functional dendrimers. Other useful building blocks are small multifunctional amine molecules such as 1,4-diaminobutane (or putrescine), 3,3′-diamino-N-methyldipropylamine, N-(3-aminopropyl)-1,3-propanediamine (or norspermidine), N-(6-aminohexyl)-1,6-hexanediamine, spermine, spermidine. Yet other useful building blocks are derivatives of amino acids such as lysine, arginine or histidine, for example dimers or trimers of L-lysine, L-histidine or L-arginine (for example, see Scheme 4), dimers or trimers of L-lysine or L-arginine, or mixed dimers or trimers of these amino acids.

Suitable examples of primary amine dendrimers are poly(propylene imine) (PPI) dendrimers or poly(amido-amine) (PAMAM) dendrimers. These type of dendrimers are known in the art. In “Materials science and technology series: Synthesis of polymers. Edited by A Dieter Schlueter, Wiley-VCH Verlag GmbH, Weinheim 1999; ISBN 3-527-26831-6; Chapter 12, pp 403-458; H M Janssen, E W Meijer, The synthesis and characterization of dendritic molecules”, the synthesis and molecular structures of PPI and PAMAM dendrimers has been described in detail.

In an embodiment of this invention, PPI-type of dendrimers are used as building blocks to prepare the polyvalent molecule.

In another embodiment of this invention, PAMAM-type of dendrimers are used as building blocks to prepare the polyvalent molecule.

Primary amine PPI-dendrimers have n-propylene-amine branching units, and have mostly been reported on with cores derived from 1,4-diamino-butane. These PPIs can be described by formula (I), where the BU is as according to formula (II-a) with p=1, and where all TUs are hydrogens. In Table 1 the features of G1 to G5 (y=1 to 5) PPI building blocks, more particularly the G1 to G5 (y=1 to 5) PPI building blocks with 1,4-diamino-butane derived cores, are compiled.

TABLE 1
Details of amine terminated PPI dendrimers
with 1,4-diaminobutane based cores.

| | PPI- | | | # Primary | # Tertiary |
|---|---|---|---|---|---|
| | Genera- | Molecular | MW | amine end | amine internal |
| Entry | tion | formula | (Dalton) | groups | groups |
| 1 | G1 | C₁₆H₄₀N₆ | 317 | 4 | 2 |
| 2 | G2 | C₄₀H₉₆N₁₄ | 773 | 8 | 6 |
| 3 | G3 | C₈₈H₂₀₈N₃₀ | 1687 | 16 | 14 |
| 4 | G4 | C₁₈₄H₄₃₂N₆₂ | 3514 | 32 | 30 |
| 5 | G5 | C₃₇₆H₈₈₀N₁₂₆ | 7168 | 64 | 62 |

Instead of using a 1,4-diamino-butane based core, one can also apply other cores for producing PPIs, such as cores derived from ammonia or bis(3-aminopropyl)amine. Table 2 highlights features of several PPI building blocks with these two cores.

TABLE 2
Details of amine terminated PPI dendrimers with ammonia
(NH₃) or bis(3-aminopropyl)amine based cores.

| | | | | | # Primary | # Tertiary |
|---|---|---|---|---|---|---|
| | PPI- | Core | Molecular | MW | amine | amine internal |
| Entry | Generation | molecule | formula | (Dalton) | end groups | groups |
| 1 | G1 | NH₃ | C₉H₂₄N₄ | 188 | 3 | 1 |
| 2 | G2 | NH₃ | C₂₇H₆₆N₁₀ | 531 | 6 | 4 |
| 3 | G3 | NH₃ | C₆₃H₁₅₀N₂₂ | 1216 | 12 | 10 |
| 4 | G1 | NH(nC3-NH₂)₂ | C₂₁H₅₂N₈ | 417 | 5 | 3 |
| 5 | G2 | NH(nC3-NH₂)₂ | C₅₁H₁₂₂N₁₈ | 988 | 10 | 8 |

Primary amine PAMAM dendrimers have N-(2-aminoethyl)propionamide branching units (BUs) and have mostly been reported on with cores derived from 1,2-diamino-ethane or ammonia. These PAMAMs can be described by formula (I), where the BU is as according to formula (II-b) with q=1 and R₁═H, and where all TUs are hydrogens. Features of several G1 and G2 PAMAM building blocks with the N-(2-aminoethyl)propionamide BU and with varying cores are compiled in Table 3.

TABLE 3
Features of amine terminated PAMAM dendrimers derived from 1,4-
diaminobutane, bis(3-aminopropyl)amine or 1,2-diaminoethane cores.
All BUs according to formula (II-b) with q = 1 and R₁ = H.

| | | | | | # Primary | # Tertiary |
|---|---|---|---|---|---|---|
| | PAMAM- | Core | Molecular | MW | amine | amine internal |
| Entry | Generation | molecule | formula | (Dalton) | end groups | groups |
| 1 | G1 | H₂N-nC4-NH₂ | C₂₄H₅₂N₁₀O₄ | 545 | 4 | 2 |
| 2 | G2 | H₂N-nC4-NH₂ | C₆₄H₁₃₂N₂₆O₁₂ | 1458 | 8 | 6 |
| 3 | G1 | NH(nC3-NH₂)₂ | C₃₁H₆₇N₁₃O₅ | 702 | 5 | 3 |
| 4 | G2 | NH(nC3-NH₂)₂ | C₈₁H₁₆₇N₃₃O₁₅ | 1843 | 10 | 8 |
| 5 | G1 | H₂N-nC2-NH₂ | C₂₂H₄₈N₁₀O₄ | 517 | 4 | 2 |
| 6 | G2 | H₂N-nC2-NH₂ | C₆₂H₁₂₈N₂₆O₁₂ | 1430 | 8 | 6 |

Similar dendrimers to PPI-dendrimers, such as poly(butylene imine) (PBuI), poly(pentylene imine) (PPeI) or poly(hexylene imine) (PHeI) dendrimers, can also be employed, especially those of lower generations G1 or G2. These dendrimers have BUs as according to formula (II-a) with p=2, 3 or 4, respectively. Scheme 8 shows examples for the preparation of a G1 poly(butylene imine) dendrimer with primary amine end groups. Repeat of the sequence of two reactions starting from the G1 material provides the G2 material. Similarly, poly(pentylene imine) or poly(hexylene imine) dendrimers can be prepared.

In an embodiment of this invention poly(butylene imine) dendrimers are used as building blocks to prepare the polyvalent molecules. In another embodiment, poly(pentylene imine) dendrimers are used as building blocks to prepare the polyvalent molecules. In another embodiment, poly(hexylene imine) dendrimers are used as building blocks to prepare the polyvalent molecules.

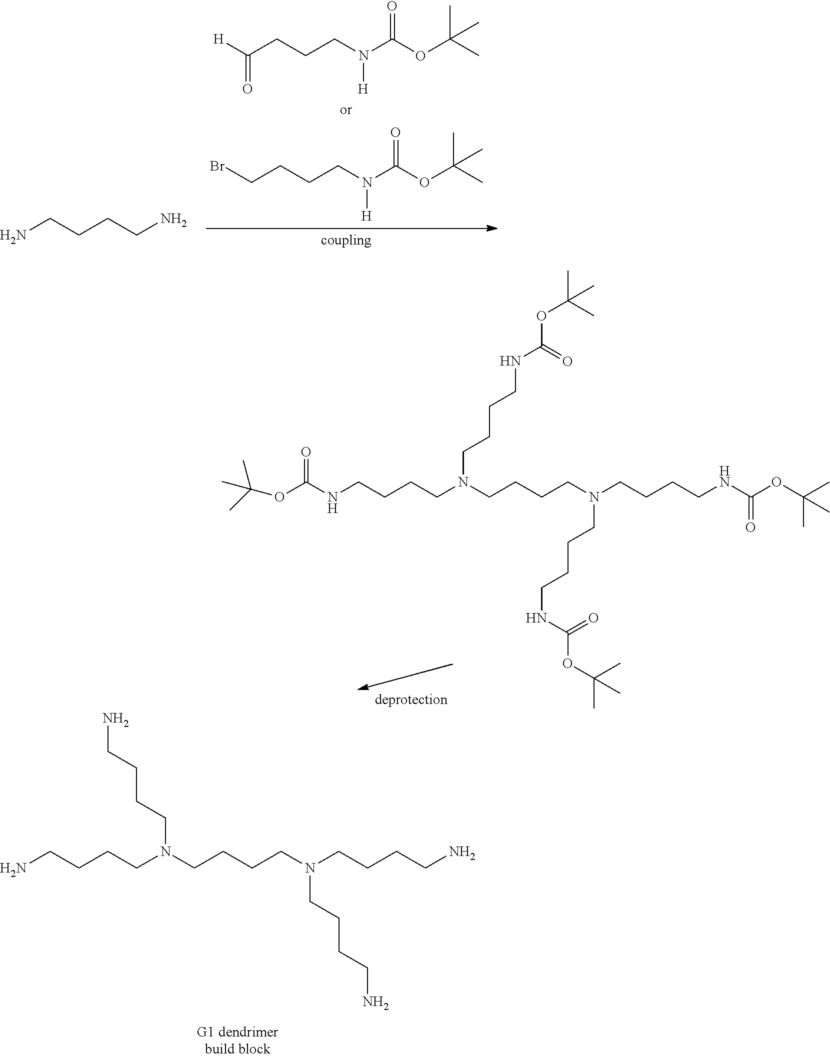 [SMILES: CC(C)(C)OC(=O)NCCCC=O ; CC(C)(C)OC(=O)NCCCCBr ; NCCCCN ; CC(C)(C)OC(=O)NCCCCN(CCCCNC(=O)OC(C)(C)C)CCCCN(CCCCNC(=O)OC(C)(C)C)CCCCNC(=O)OC(C)(C)C ; NCCCCN(CCCCN)CCCCN(CCCCN)CCCCN]

Scheme 8: Sequence of Two Reaction Steps from Putrescine to Produce a G1 Poly(Butylene Imine) (PBuI) Dendrimer Building Block. The Bromide Reactant Couples in an Alkylation Reaction; Alternatively, the Aldehyde Reactant Couples in a Reductive Amination. Deprotection of the Boc-Groups Produces the Amine Functional PBuI of G1.

Next to PAMAM dendrimers with N-(2-aminoethyl)propionamide BUs, similar PAMAM dendrimers with other BUs can be employed, especially those of lower generations G1 or G2. Scheme 9 shows an example for the preparation of a G1 PAMAM dendrimer in which the BU is as according to formula (II-b) with q=2 and R₁=Me. Repeat of the sequence of two reactions starting from the G1 material provides the G2 material.

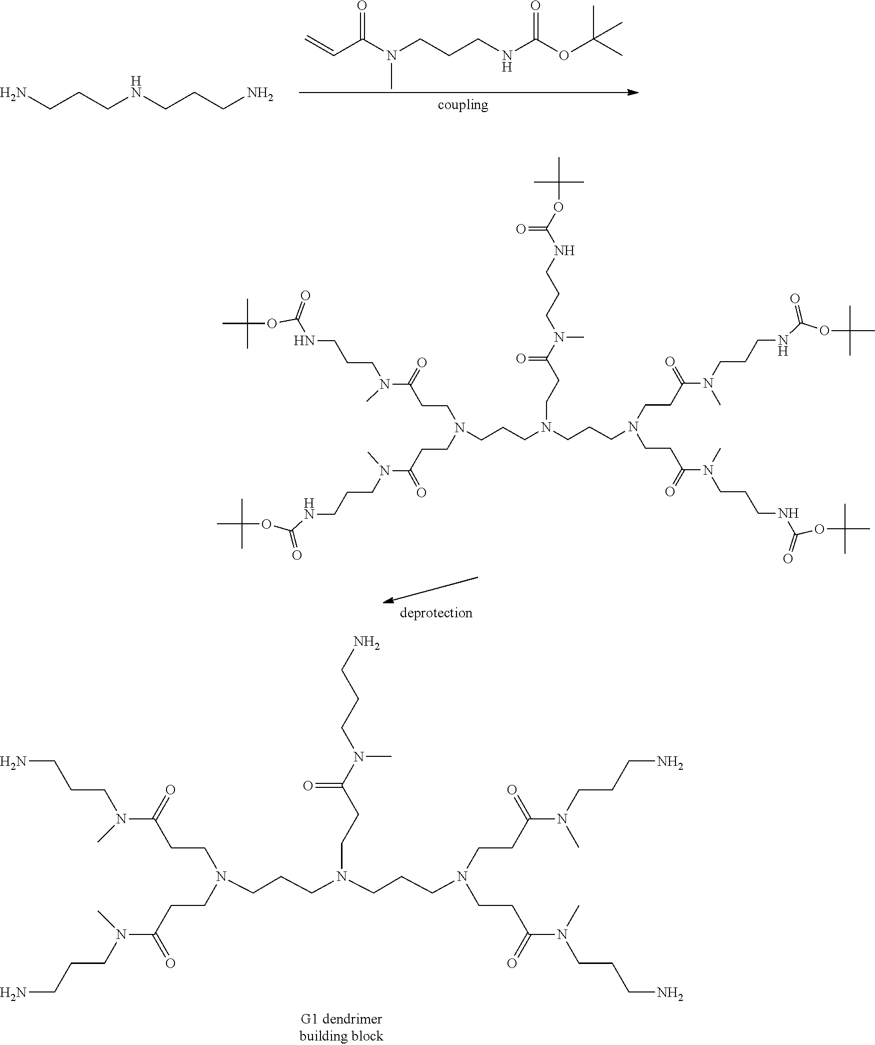 [SMILES: C=CC(=O)N(C)CCCNC(=O)OC(C)(C)C ; NCCCNCCCN ; CN(CCCNC(=O)OC(C)(C)C)C(=O)CCN(CCCN(CCC(=O)N(C)CCCNC(=O)OC(C)(C)C)CCC(=O)N(C)CCCNC(=O)OC(C)(C)C)CCCN(CCC(=O)N(C)CCCNC(=O)OC(C)(C)C)CCC(=O)N(C)CCCNC(=O)OC(C)(C)C ; CN(CCCN)C(=O)CCN(CCCN(CCC(=O)N(C)CCCN)CCC(=O)N(C)CCCN)CCCN(CCC(=O)N(C)CCCN)CCC(=O)N(C)CCCN]

Scheme 9: Sequence of Two Reaction Steps from Bis(3-Aminopropyl)Amine to Produce a G1 PAMAM Dendrimer Building Block. The Acrylamide Reactant Couples in a Michael-Addition Reaction. Deprotection of the Boc-Groups Produces the Amine Functional PAMAM of G.

Further G1 PAMAM building blocks with varying BUs can be prepared in a similar synthetic approach, and some examples are highlighted in Table 4 (using a 1,4-diaminobutane based core) and in Table 5 (using a bis(3-aminopropyl)amine based core).

TABLE 4
Features of G1 amine terminated PAMAM dendrimers derived from a 1,4-
diaminobutane core, applying the indicated branching units BU.

| | | | | | # Primary | # Tertiary |
|---|---|---|---|---|---|---|
| | PAMAM | BU of | Molecular | MW | amine | amine internal |
| Entry | Gen. | formula (II-b) | formula | (Dalton) | end groups | groups |
| 1 | G1 | R₁ = H, q = 2 | C₂₈H₆₀N₁₀O₄ | 601 | 4 | 2 |
| 2 | G1 | R₁ = CH₃, q = 2 | C₃₂H₆₈N₁₀O₄ | 657 | 4 | 2 |
| 3 | G1 | R₁ = H, q = 3 | C₃₂H₆₈N₁₀O₄ | 657 | 4 | 2 |
| 4 | G1 | R₁ = H, q = 5 | C₄₀H₈₄N₁₀O₄ | 769 | 4 | 2 |

TABLE 5
Features of G1 amine terminated PAMAM dendrimers derived from a bis(3-
aminopropyl)amine core, applying the indicated branching units BU.

| | | | | | # Primary | # Tertiary |
|---|---|---|---|---|---|---|
| | PAMAM | BU of | Molecular | MW | amine | amine internal |
| Entry | Gen. | formula (II-b) | formula | (Dalton) | end groups | groups |
| 1 | G1 | R₁ = H, q = 2 | C₃₆H₇₇N₁₃O₅ | 772 | 5 | 3 |
| 2 | G1 | R₁ = CH₃, q = 2 | C₄₁H₈₇N₁₃O₅ | 842 | 5 | 3 |
| 3 | G1 | R₁ = H, q = 3 | C₄₁H₈₇N₁₃O₅ | 842 | 5 | 3 |
| 4 | G1 | R₁ = H, q = 5 | C₅₁H₁₀₇N₁₃O₅ | 983 | 5 | 3 |

For preparing the polyvalent molecule, lower generation dendrimers are preferred. Accordingly, G1, G2 or G3 dendrimers are preferred. More preferred are G1 PPI, G1 PBuI, G1 PPeI or G1 PAMAM dendrimers and G2 PPI or G2 PAMAM dendrimers.

Other building blocks for preparing the polyvalent molecule are small multifunctional amines such as N-(3-aminopropyl)-1,3-propanediamine (or norspermidine), N-(6-aminohexyl)-1,6-hexanediamine, spermine, spermidine. These molecules can for example and preferably be used to connect directly to TUs.

Next, the multifunctional amine building block—e.g a small multifunctional amine molecule, a PPI (or PBuI or PPeI or PHeI) dendrimer, or a PAMAM dendrimer—is converted with a reactant, or with several reactants, to acquire the polyvalent molecule. The following non-limiting examples illustrate how this can be done; see also Scheme 6 for the indicated options for formula types (III).

Introduction of TUs of type (III-a). When the amine functional building block is left unreacted for (a minor) part of the primary amine reactive groups, or when the reaction is incomplete, then —NH₂ or —NHR hydrogens remain untouched. This is one way to acquire hydrogen TUs in the polyvalent molecule.

Introduction of TUs of types (III-b), (III-e) or (III-f). The amine functional building block can be alkylated or alkylene-arylated, e.g. with alkyl-halides, alkyl-tosylates or benzyl-halides that optionally contain O and/or N heteroatoms. Alternatively, alkylation or alkylene-arylation can be achieved by reductive amination reactions applying aldehyde or ketone functional molecules such as e.g. alkyl-aldehydes or benzyl-aldehydes.

Introduction of TUs of type (III-c). The amine functional building block can be coupled in Michael additions to acryl-esters or methacryl-esters, preferable acryl-esters. Typically, these esters have alkyl chains, that may be linear or branched, and saturated or unsaturated. Examples are ethyl-acrylate, n-butyl-acrylate, n-octyl-acrylate, 2-ethylhexyl-acrylate, n-dodecyl-acrylate, iso-decyl-acrylate or citronellyl-acrylate, preferably ethyl-acrylate, n-butyl-acrylate, n-octyl-acrylate, n-dodecyl-acrylate, iso-decyl-acrylate or citronellyl-acrylate. Preferred acryl-esters are C6-C12 acryl-esters; the C6-C12 alkyl groups may be linear or branched, and saturated or unsaturated. We have found that acryl-esters react (considerably) faster with primary amines than with secondary amines (i.e. selective reaction). Accordingly, this can be employed to first react primary amine molecules with 1 molar equivalent of an acryl-ester to convert all primary amines to secondary amines. The remaining secondary amines can then be converted by e.g. acylation, alkylation, reductive amination, etc. It is also possible to convert all the secondary amines with acryl-esters, simply by using a molar excess of acryl-ester reactant. It is also possible to first introduce a first acryl-ester, and then a second different one. Preferably, the amine functional building block is reacted with one acryl-ester only. Such reaction may lead to full conversion of both the primary and secondary amines with this specific acryl-ester.

Introduction of TUs of type (III-d). The amine functional building block can be coupled in Michael additions to acryl-amides or methacryl-amides, preferably acryl-amides. Typically, these amides have alkyl chains, that may be linear or branched, and saturated or unsaturated, and they may have functional groups such as alcohols. Examples are n-butyl-acrylamide, n-octyl-acrylamide, n-decyl-acryl-amide, N,N-diethylacrylamide, N,N-dibutyl-acrylamide, N-methyl-N-hexyl-acrylamide and N-(2-hydroxyethyl)-N-methyl acrylamide, preferably n-butyl-acrylamide, n-octyl-acrylamide, n-decyl-acryl-amide, N,N-diethylacrylamide, N,N-dibutyl-acrylamide, and N-methyl-N-hexyl-acrylamide. Acryl-amides typically react faster with primary amines than with secondary amines. This selectivity can be employed to first react primary amine molecules with acryl-amide to convert all primary amines to secondary amines. The remaining secondary amines can then be converted by e.g. acylation, alkylation, reductive amination, etc.

Alternatively, the amine functional building block is first reacted with an acryl-ester (or a methacryl-ester), and subsequently these esters are amidated by reaction with primary or secondary amines. The esters may also be hydrolysed and then amidated with primary or secondary amines.

Introduction of TUs of type (III-g). The amine functional building block can be alkylated by using 1,2-epoxy-alkanes, such as for example 1,2-epoxy-hexane, 1,2-epoxy-octane, 1,2-epoxy-decane or 1,2-epoxy-dodecane. The alkanes may be linear or branched, and saturated or unsaturated. Reaction of amines with these molecules gives introduction of both an alkyl chain and an alcohol group. Preferably, the amine functional building block is reacted with one 1,2-epoxy-alkane only, leading to full conversion of both the primary and secondary amines with this specific 1,2-epoxyalkane. Optionally, in a further reaction step, the created secondary alcohols can be acylated by reaction with carboxylic acids, active esters or anhydrides, such as for example acetic anhydride.

Introduction of TUs of types (III-h), (III-i) and (III-a). The amine functional building block can be acylated by reaction with e.g. carboxylic acids, active esters or anhydrides. Using this chemistry, a primary amine is converted to an amide, implying that TUs of type (III-h) and of type (III-a) are simultaneously generated (or of type (III-i) and (III-a)). Introduced alkanes may be linear or branched, and saturated or unsaturated.

Introduction of TU of type (III-j) and (III-a). The amine functional building block can be reacted with guanidinylation agents. These agents react with primary or secondary amines to furnish a guanidine group. Examples of such agents are pyrazole-type guanidinylation agents such as 1-amidinopyrazole hydrochloride, 1-carbamimidoyl-1,2,4-triazole hydrochloride, 1-(N-Boc-amidino)-pyrazole, 1-(N-Cbz-amidino)-pyrazole, or other molecules such as N,N′-di-Boc-S-methyl-iso-thiourea and N-Boc-S-methyl-iso-thiourea. Deprotection of the Boc or Cbz groups creates the guanidine group. Using this chemistry, TUs of type (III-j) and of type (III-a) may be simultaneously introduced at every primary amine end group.

One of the above conversion methods, or a combination of the above methods, can be employed to prepare the polyvalent molecule of choice.

Cationic groups can be introduced into the polyvalent molecule by quaternizing tertiary amines, guanidines or imidazoles. In a non-limiting example, all primary amines in a PPI (or a PBuI or a PPeI or a PHeI) or a PAMAM dendrimer building block can first be protected with e.g. a Boc-group. This gives molecules such as those shown in Schemes 8 and 9. Next, the interior tertiary amines can be quaternized to ammonium groups by reaction with alkylating agents such as e.g. methyl-iodide or benzyl-halides. Deprotection of the Boc-groups gives a PPI (or a PBuI or a PPeI or a PHeI) or a PAMAM building block with internal quaternary ammonium cationic groups as well as outer primary amines. These outer amines can then be converted with amine-reactive molecules to acquire the polyvalent molecule, more particularly the polyvalent molecule with cationic groups at the interior of the molecule.

Formulas (IV-A) to (IV-F) in Scheme 10 show various non-limiting embodiments for the polyvalent molecule, more particularly the polyvalent molecule as according to Formula (I). Formulas (IV-A) and (IV-B) show G0 materials (i.e. no BUs), formulas (IV-C) and (IV-D) show first generation G1 dendrimers (i.e. 1 BU-layer), formula (IV-E) shows a second generation G2 dendrimer (2 BU-layers) and formula (IV-F) shows a third generation (G3) dendrimer (3 BU-layers).

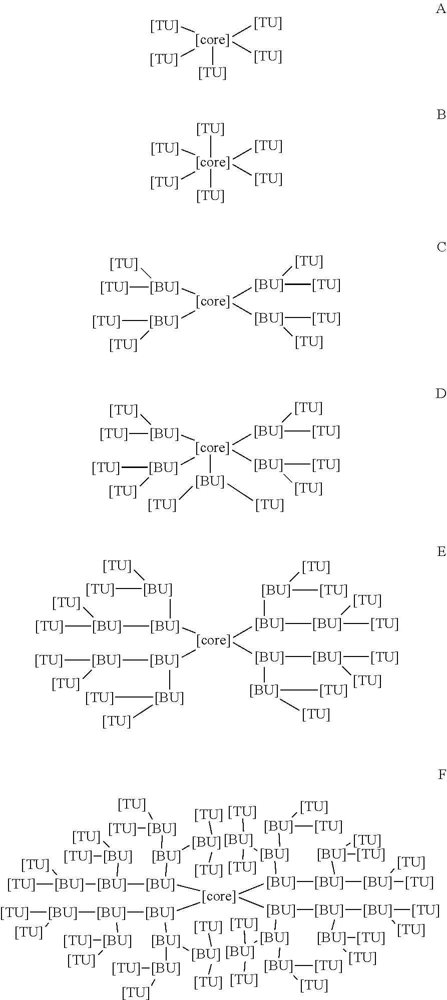

### Scheme 10: Polyvalent Molecules as According to Formulas (IV-a) to (IV-F). (IV-A): x=5, y=0, z=5; (IV-B): x=6, y=0, z=6; (IV-C): x=4, y=1, z=8; (IV-D): x=5, y=1, z=10; (IV-E): x=4, y=2, z=16; (IV-F): x=4, y=3, z=32

In a further embodiment, the polyvalent molecules as according to formulas (IV-C) to (IV-F) are selected from poly(propylene imine) (PPI) dendrimers and PAMAM dendrimers, or from modifications thereof. Scheme 11 shows examples of PPI dendrimers of the first G1, second G2 and third G3 generations with cores derived from 1,4-diaminobutane. Scheme 12 shows examples of PAMAM dendrimers of the first G1 and second G2 generations with cores derived from 1,2-diaminoethane. Poly(butylene imine) (PBuI) dendrimers, poly(pentylene imine) (PPeI) dendrimers, poly(hexylene imine) (PHeI) dendrimers can also be employed. Scheme 13 shows G1 examples of PBuI, PPeI and PHeI dendrimers with cores derived from 1,4-diaminobutane.

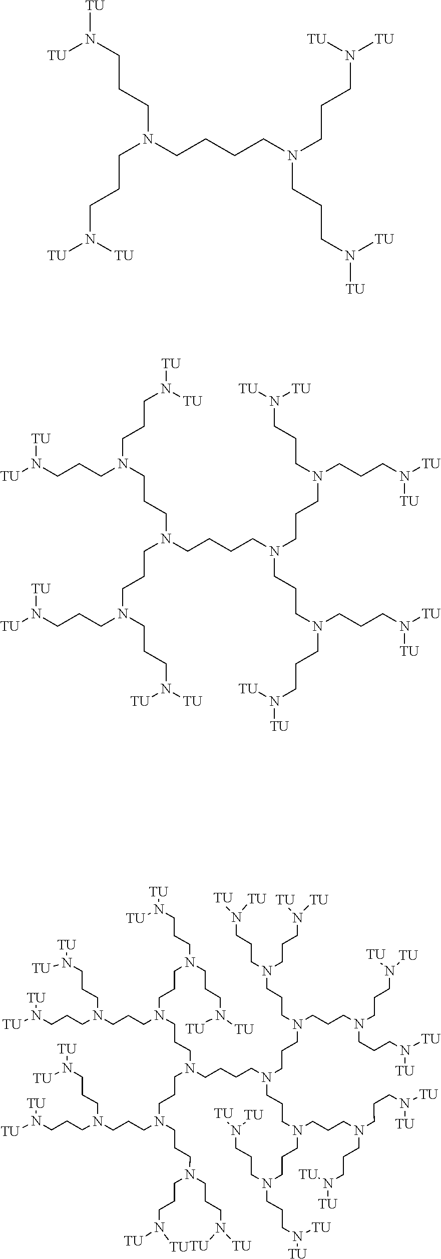

Scheme 11: G1, G2 and G3 PPI Dendrimers (Counterclockwise from Top Left) With n-butylene-Diamine Derived Cores (ρ=1 in Formula IIa).

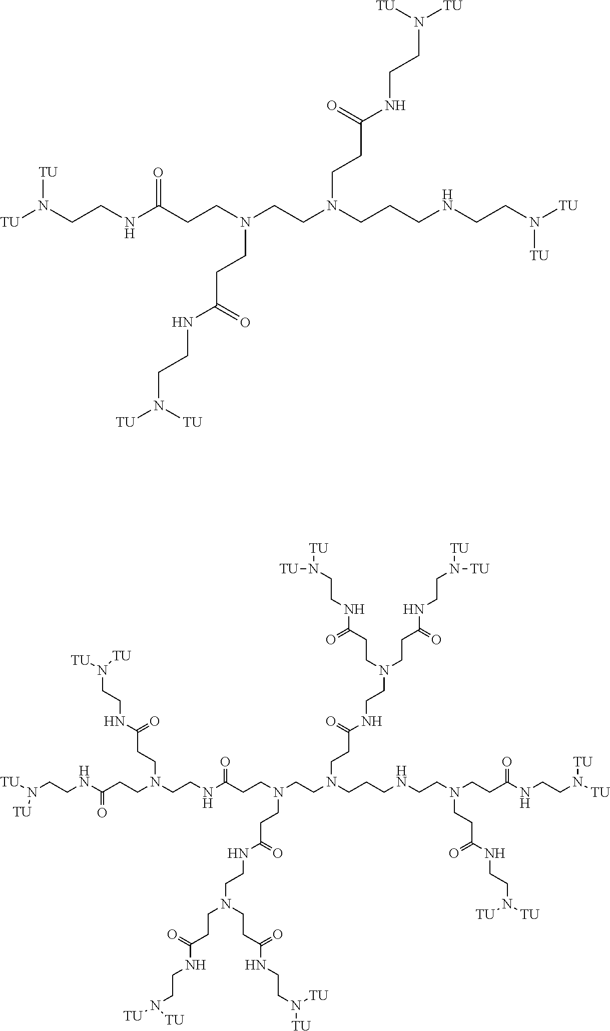

Scheme 12: G1 and G2 PAMAM Dendrimers with n-Ethylene-Diamine Derived Cores (q=1 and R₁═H in formula IIb).

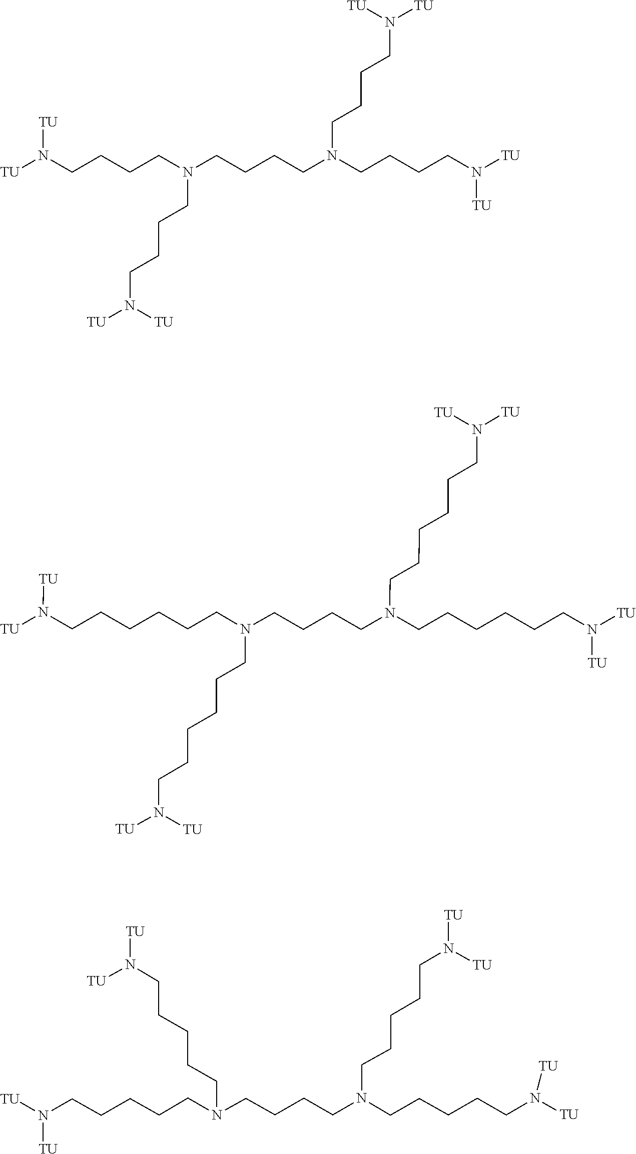

Scheme 13: G1 PBuI, G1 PPeI and G1 PHeI Dendrimers with n-Butylene-Diamine Derived Cores (p=2, 3 and 4 in Formula La, Respectively).
b. The stabiliser

The stabiliser has the function to stabilise the nanoparticle, and can serve as for example as an agent to prevent aggregating or to prevent decay of the nanoparticles. The stabiliser may be a protein, a polysaccharide or other (macro)molecule, or a conjugate of a protein, a polysaccharide or other (macro)molecule with for example a component of the nanoparticle (for example conjugated to a phospholipid or lipid). The stabiliser may further be used to control the size of the nanoparticle and to increase its shelf life. Non limiting examples of suitable stabilisers are an apolipoprotein, an apolipoprotein derivative, an apolipoprotein mimetic and/or an apolipoprotein mimetic derivative. Other non-limiting examples of stabilisers are a hydrophilic polymer or a hydrophilic polymer modified molecule, such as a polyethylene glycol (PEG) or polyethylene glycol modified molecule; a polysaccharide or polysaccharide modified molecule; a polysarcosine or polysarcosine modified molecule; or a poly(2-oxazoline) or poly(2-oxazoline) modified molecule.

In principle the stabiliser is predominantly present on the outer layer of the particle. However, it is understood that the stabiliser may be a conjugate, for example of PEG and a (phospho)lipid, in which case the PEG is predominantly present in the water layer immediately surrounding the nanoparticle, while the conjugated lipid resides predominantly in the outer layer of the nanoparticle, presumably next to other (phospho)lipids that are not modified with PEG.

When the stabiliser contains non-natural synthetic groups, such as for example one of the forementioned polyethylene glycol, polysarcosine or poly(2-oxazoline) polymer chains, this may be less desirable, as such groups may invoke an immune response upon (frequently) repeated administration. Furthermore, such polymer groups usually have a non-fouling character, implying that nanoparticles that expose such groups may experience a barrier when interacting with cells. Accordingly, protein or peptide-based stabilisers are preferred.

In a particular embodiment the stabiliser is an apolipoprotein, an apolipoprotein derivative, an apolipoprotein mimetic and/or an apolipoprotein mimetic derivative. Apolipoproteins are natural helical proteins with inherent affinity for (phospho)lipid layers due to their architecture and amphiphilic character; these materials, however, are not traditional hydrophilic head/hydrophobic tail amphiphiles. There are several classes of apolipoproteins, and all can be used as a structural component for nanoparticle formulations. Apolipoprotein integration affects the nanoparticle's physicochemical properties and shelf-life by providing structural stability. Furthermore, the presence of apolipoprotein modulates the biological behavior of the nanoparticle. For example, apolipoprotein A1 interacts with cells via scavenger receptor class B type 1 (SRB1) and ATP-binding cassette transporter ABCA1. This increases interactions of the nanoparticle with myeloid cells in lymphoid organs.

It was found that by using an apolipoprotein, for example apo A1, the nanoparticle can successfully be targeted to specific cells, tissues, or organs, for example but not limiting to the myeloid compartment. This has the advantage that specific cells in the subject can be targeted by drugs in order to stimulate or inhibit an response. For example, immune cells can be targeted to stimulate or inhibit an immune response. There are several therapeutical applications where such use is deemed beneficial, such as but not limited to cancer, cardiovascular disease, autoimmune disorders, and xenograft rejection.

In particular embodiment, the apolipoprotein, apolipoprotein derivative, apolipoprotein mimetic and/or apolipoprotein mimetic derivative is an apolipoprotein, an apolipoprotein derivative, an apolipoprotein mimetic and/or an apolipoprotein mimetic derivative targeting the myeloid cell compartment, preferably apoA1 or a mimetic or derivative thereof.

Because the nanoparticle as described herein, when using apolipoprotein as a stabiliser, has an exterior which is identical to an HDL particle, the nanoparticle will not trigger an immune response which may result in premature degradation or clearance of the nanoparticle by the immune system prior to reaching its intended target, e.g. the myeloid compartment.

The term apolipoprotein when used herein refers to a protein that together with lipids forms lipoproteins, i.e. an assembly of lipids and proteins. Apolipoproteins typically function to transport lipids and fat-soluble substances in the blood. Apolipoproteins are described and include but are not limited to apo A1, apo A1-Milano, apo A2, apo A4, apo A5, apo B48, apo B100, apo C-I, apo C-II, apo C-III, apo C-IV, apo D, apo E, apo F, apo H, apo L and apo M. In particular embodiments, the apolipoprotein stabiliser or component may be an apolipoprotein derivative. An apolipoprotein derivative may be a simple modifications of apolipoproteins, for example produced in one-step conversions from the protein. When used herein, the term apolipoprotein may further refer to apolipoprotein mimetics. Apolipoprotein mimetics are short peptides that mimic the properties of an apolipoprotein. An example of an apo A1 mimetic peptide is usually referred to as 18A, which is DWLKAF YDKVAE KLKEAF (SEQ ID NO: 1), with an unfunctionalized N-terminus and C-terminus. Another reported, more convenient, and also more active mimetic is 2F, which 18A (SEQ ID NO: 1) with an acetamide capped N-terminus and an amide C-terminus. In Leman, L. J. et al., J. Med. Chem. 2014, 57, 2169-2196 (10.1021/jm4005847) more examples of apo A1 peptidomimetics are reported, particularly in Table 2 and Table 3. Apo A1 peptidomimetic derivatives, such as dimer, trimer and tetramer peptides are illustrated in Zhou et al., J. Am. Chem. Soc. 2013, 135, 13414-13424 (dx.doi.org/10.1021/ja404714a). Preferred apo A1 peptidomimetics or peptidomimetics derivatives are 18A, 2F and 4F, and any dimers of these peptides. Preferred apoA1 peptidomimetics are 18A, 2F and 4F, and any dimers of these peptides. More preferred are 2F and any dimers or trimers of this peptide.

Apolipoproteins are proteins that bind lipids to form lipoproteins. They transport lipids and fat-soluble vitamins in blood, cerebrospinal fluid, and lymph. The lipid components of lipoproteins are insoluble in water. However, because of their amphipathic properties, apolipoproteins, and other amphipathic molecules such as phospholipids can surround the lipids, creating a lipoprotein particle that is itself water-soluble, and can thus be carried through water-based circulation (i.e., blood, extracellular fluids, lymph). In addition to stabilizing lipoprotein structure and solubilizing the lipid component, apolipoproteins interact with lipoprotein receptors and lipid transport proteins, thereby participating in lipoprotein uptake and clearance. They also serve as enzyme cofactors for specific enzymes involved in the metabolism of lipoproteins.

Apolipoprotein A1 is a protein that in humans is encoded by the APOA1 gene. As the major component of HDL particles, it has a specific role in lipid metabolism. The protein, as a component of HDL particles, enables efflux of fat molecules by accepting fats from within cells (including macrophages within the walls of arteries which have become overloaded with ingested fats from oxidized LDL particles) for transport (in the water outside cells) elsewhere, including back to LDL particles or to the liver for excretion.

It is envisioned that any apolipoprotein may be used in the nanoparticles. Therefore, in an embodiment the apolipoprotein is selected from apo A1, apo A1-Milano, apo A2, apo A4, apo A5, apo B48, apo B100, apo C-I, apo C-II, apo C-III, apo C-IV, apo D, apo E, apo F, apo H, apo L and apo M, and combinations thereof, preferably selected from apo A1, apo A2, apo A4, apo A5, apo B100, apo C-I, apo C-II, apo C-III, apo C-IV and apo E, more preferably selected from apo A1, apo A4, apo A5, apo B100, apo C-III and apo E, even more preferably selected from apo A1, apo B100 and apo E. In a particularly preferred embodiment the apolipoprotein is apo A1 because it allows targeting of the nanoparticle to the myeloid compartment. In an alternative preferred embodiment the apolipoprotein is apo E because it allows targeting of the nanoparticle to dendritic cells.

Apolipoproteins can be produced and purified by methods that are known in the art, such as recombinant protein expression from E-coli bacteria, or from other organisms, followed by steps required to isolate the apo A1 is sufficiently pure form.

It was found by the inventors that there are several advantages associated with the use of apolipoprotein in nanoparticles to deliver a nucleic acid at a target site. First of all, apolipoprotein stabilises the nanoparticles by preventing aggregation during preparation and storage. For the nanoparticles to stay in a stable emulsion it is essential that the nanoparticles do not aggregate or fuse, which may result in precipitation of the particles. The apolipoprotein helps to stabilise the particles and prevents aggregation. Further, apolipoprotein ensures in vivo stability of the nanoparticles. Because apolipoprotein is naturally present on lipid particles circulating in the blood stream, such as LDL and HDL, they are not recognized by the immune system as non-self, thereby ensuring natural stealth, as opposed to chemical modifications or other non-natural methods to improve stability. Lastly, the use of apolipoprotein facilitates desirable interactions with immune cells, for example in the myeloid compartment to deliver the nucleic acid cargo.

Therefore, in an embodiment of the invention the stabiliser, e.g. an apolipoprotein component such as an apolipoprotein, an apolipoprotein derivative, an apolipoprotein mimetic and/or an apolipoprotein mimetic derivative, in the nanoparticle is used to:
prevent aggregation upon preparation and storage;
improve in vivo stability;
provide natural stealth; and/or
facilitate targeting to and/or interactions with immune cells.

In an embodiment, the stabiliser as described herein is selected from:
an apolipoprotein, an apolipoprotein derivative, an apolipoprotein mimetic and/or an apolipoprotein mimetic derivative;
a polyethylene glycol or polyethylene glycol modified molecule;
a hydrophilic polymer or hydrophilic polymer modified molecule;
a polysaccharide or polysaccharide modified molecule;
a polysarcosine or polysarcosine modified molecule; or,
a poly(2-oxazoline) or poly(2-oxazoline) modified molecule, or a combination thereof.

Preferably, the stabiliser is an apolipoprotein, an apolipoprotein derivative, an apolipoprotein mimetic and/or an apolipoprotein mimetic derivative, or a polyethylene glycol modified molecule, or a combination thereof. More preferably, the stabiliser is an apolipoprotein, an apolipoprotein derivative, an apolipoprotein mimetic and/or an apolipoprotein mimetic derivative. Most preferably, the stabiliser is an apolipoprotein. In particular embodiments, the apolipoprotein, apolipoprotein derivative, apolipoprotein mimetic and/or apolipoprotein mimetic derivative is located on the outer surface of the outer layer of the nanoparticle.

In particular embodiments, the apolipoprotein stabiliser as described elsewhere herein may be fused to a targeting body that allows targeting the nanoparticle as taught herein to a different target than it would bind when the apolipoprotein stabiliser was not fused to the targeting body (or in other words, a different target than that to which it would have innately bound), and/or to bind to its intended target with a higher affinity, and may as a result reduce off-target effects. For example, the targeting body may allow the nanoparticle to bind a non-myeloid cell, such as a lymphocyte, like a T cell, a B cell or a natural killer (NK) cell, or an endothelial cell, or to a myeloid cell with higher affinity.

The targeting body may be an antibody or an antigen binding fragment thereof, a rerouting peptide or a rerouting protein, such as receptor binding peptides, ligand mimicking peptides, a receptor ligand, a receptor, or interacting protein. It is envisioned that any type of antigen binding molecule can in principle be used as a targeting body. Non-limiting examples of targeting bodies include programmed cell death protein 1 (PD1), signal-regulatory protein alpha (SIRPa). CD40L, GP120, an antibody or antigen binding fragment thereof (specifically) binding to CD8 (e.g. VHHCD8), a targeting body capable of binding to Factor VIII-related antigen such as Factor VIII, a targeting body capable of binding to CD31/PECAM-1 such as CD31, a targeting body capable of binding to Angiotensin-converting enzyme (ACE/CD143) such as angiotensin, a targeting body capable of binding to CD34 such as L-selectin or a targeting body capable of binding to endoglin (CD105).

In the embodiment where the stabiliser is derived from a hydrophilic polymer, a polyethylene glycol (PEG), a polysaccharide, a polysarcosine or a poly(2-oxazoline), then these polymers have been modified with hydrophobic moieties. These can for example be hydrophobic polymers, hydrophobic C12-18 alkyl chains or phospholipids with chains derived from C14, C16 or C18 fatty acids. The dimyristoyl phosphatidylethanolamine (DMPE), dipalmitoyl phosphatidylethanolamine (DPPE) and distearoyl phosphatidylethanolamine (DSPE) phospholipids are particularly useful for this purpose, as they are primary amine functional, so they can be connected to oligomeric or polymeric units of PEG, polysaccharide, polysarcosine or poly(2-oxazoline. The molecular weights of the oligomers or polymers can be about 200 Dalton or higher, about 500 Dalton or higher, about 1000 Dalton or higher, or about 2000 Dalton or higher. At lower molecular weights, the oligomers may be discrete. For example, DPPE or DSPE may be connected to an oligo-ethylene glycol chain with 4, 6, 12 or 24 ethylene glycol units. Typically, the oligo-glycols or PEGs are capped with methoxy or hydroxy groups.

c. The Nucleic Acid

The purpose of the nanoparticles described herein is to deliver a nucleic acid to a cell. The nucleic acid may be for example an mRNA encoding a peptide or protein of interest which is to be expressed in the cell, or may comprise a short nucleic acid such as an siRNA, shRNA intended to interfere in gene expression (e.g. gene silencing), or it may comprise a component of the CRISPR-Cas or a related system to induce a mutation in the genome of the cell. Therefore, in general the mode of action of the nucleic acid (the payload of the nanoparticle) is in the nucleus. Therefore, the nanoparticle preferably has at least the following properties:
1) it allows targeting of the intended target cell, and
2) it allows delivery of the payload where it can assert its action (thus in most cases in the nucleus of the target cell).

Without wishing to be bound to theory, it is thought that the polyvalent molecules described herein play a role in the endosomal escape of the payload once the nanoparticles are absorbed by the target cell and thus allow nuclear delivery of the nucleic acid payload.

Many different types of RNA, DNA or synthetic oligonucleotides have been used as nucleic acid therapeutic. The present invention is not limited to a specific type of nucleic acid as the invention is envisioned to work with any type that can be loaded using a polyvalent molecule in the nanoparticles. Therefore, in an embodiment, the nucleic acid is RNA, DNA, or a nucleic acid analogue,
preferably wherein the RNA is microRNA (miRNA), small interfering RNA (siRNA), piwi-interacting RNA (piRNA), small nuclear RNA (snoRNA), transfer RNA (tRNA), tRNA-derived small RNA (tsRNA), small regulatory RNA (srRNA), messenger RNA (mRNA), modified mRNA, ribosomal RNA (rRNA), long non-coding RNA (lncRNA) or guide RNA (gRNA) or combinations thereof and/or modifications thereof; or
preferably wherein the DNA is single stranded or double stranded DNA; or
preferably wherein the antisense oligonucleotide is single strand DNA or RNA consisting of nucleotide or nucleoside analogues containing modifications of the phosphodiester backbone or the 2′ ribose, more preferably wherein the nucleotide or nucleoside analogues are selected from locked nucleic acid (LNA), bridged nucleic acid (BNA), morpholino or peptide nucleic acid (PNA).
   d. The Sterol

When used herein the term sterol refers to compounds that are derived from sterol (2,3,4,5,6,7,8,9,10,11,12,13,14,15,16,17-hexadecahydro-1H-cyclopenta[a]phenanthren-3-ol) by substituting other chemical groups for some of the hydrogen atoms, or modifying the bonds in the ring. Sterols and related compounds play essential roles in the physiology of eukaryotic organisms. For example, cholesterol forms part of the cellular membrane in animals, where it affects the cell membrane's fluidity and serves as secondary messenger in developmental signalling.

When used herein, sterol may for example refer to cholesterol, ergosterol, hopanoids, hydroxysteroid, phytosterol, steroids, hydrogenated cholesterol, or zoosterol. In the nanoparticle the sterol maintains or regulates the fluidity in the membrane (i.e. in the phospholipid monolayer surface barrier).

In an embodiment the sterol is selected from cholesterol, stigmasterol, or β-sitosterol, or combinations thereof. In an embodiment the sterol is selected from: cholesterol, ergosterol, hopanoids, hydroxysteroid, phytosterol, steroids, zoosterol, stigmasterol, or β-sitosterol, or combinations thereof. In a preferred embodiment the sterol is or comprises cholesterol.

e. The Phospholipid

Phospholipids, also known as phosphatides, are a class of lipids whose molecule has a hydrophilic head containing a phosphate group, and two hydrophobic tails derived from fatty acids, joined by a glycerol molecule. Marine phospholipids typically have omega-3 fatty acids EPA and DHA integrated as part of the phospholipid molecule. The phosphate group can be modified with simple organic molecules such as choline, ethanolamine, or serine.

Phospholipids are a key component of all cell membranes. They can form lipid bilayers because of their amphiphilic characteristic. In eukaryotes, cell membranes also contain another class of lipid, a sterol, that is interspersed among the phospholipids. The combination provides fluidity in two dimensions combined with mechanical strength against rupture.

Therefore, in an embodiment the phospholipid is selected from a phosphatidylcholine, a phosphatidylethanolamine, a phosphatidylserine and a phosphatidylglycerol, or combinations thereof.

The acyl groups in the phospholipid may, individually, be derived from medium chain or long chain fatty acids. In an embodiment at least one, preferably both, of the acyl groups in the phospholipid are derived from long chain fatty acids, preferably wherein said long chain fatty acids are selected from C14, C16 or C18 chains, i.e. from myristic acid, myristoleic acid, palmitic acid, palmitoleic acid, stearic acid, oleic acid and linoleic acid, or combinations thereof.

Lyso-phospholipids are phospholipids in which one of the acyl groups has been removed by hydrolysis, leaving an alcohol group. These molecules therefore have one instead of two fatty acid chains. These phospholipids can also be applied, for example to regulate the shape, function, and fluidity of the outer layers of the nanoparticle.

In a particularly preferred embodiment, the phospholipid is a neutral phospholipid, meaning it is zwitterionic at physiological pH (it has a nett neutral charge). Therefore, in a preferred embodiment the phospholipid is a phosphatidylcholine (PC) or a phosphatidylethanolamine (PE).

Accordingly, examples of phospholipids that can be used are dilauroylphosphatidylcholine (DLPC), dimyristoylphosphatidylcholine (DMPC), dipalmitoylphosphatidylcholine (DPPC), distearoylphosphatidylcholine (DSPC), dioleoylphosphatidylcholine (DOPC), dilauroylphosphatidylglycerol (DLPG), dimyristoylphosphatidylglycerol (DMPG), dipalmitoylphosphatidylglycerol (DPPG), distearoylphosphatidylglycerol (DSPG), dioleoylphosphatidylglycerol (DOPG), dilauroyl phosphatidylethanolamine (DLPE), dimyristoyl phosphatidylethanolamine (DMPE), dipalmitoyl phosphatidylethanolamine (DPPE), distearoyl phosphatidylethanolamine (DSPE), dilauroyl phosphatidylserine (DLPS), dimyristoyl phosphatidylserine (DMPS), dipalmitoyl phosphatidylserine (DPPS), distearoyl phosphatidylserine (DSPS), 1-palmitoyl-2-oleoyl-sn-glycero-3-phosphocholine (POPC), 1,2-dioleoyl-sn-glycero-3-phosphoethanolamine (DOPE), as well as mixtures thereof.

As lysophospholipids, 1-myristoyl-2-hydroxy-sn-glycero-3-phosphocholine (MHPC), 1-palmitoyl-2-hydroxy-sn-glycero-3-phosphocholine (PHPC) and 1-stearoyl-2-hydroxy-sn-glycero-3-phosphocholine (SHPC), or mixtures thereof can be employed.

f. The Filler Molecule

Nanoparticles as described herein may further, and optionally, comprise a filler molecule. Filler molecules are biocompatible molecules such as (any kind of) esters or amides. Esters are preferred, especially those derived from fatty acids or cholesterol, wherein the fatty acids may have saturated or unsaturated chains. Filler molecules are hydrophobic in nature and can for example be lipids, such as, but not limited to, triglyceride lipids. Therefore, in an embodiment the nanoparticle further comprises a filler molecule selected from a triglyceride, a diglyceride, an ester derived from a fatty acid, and a cholesteryl ester, or combinations thereof.

The nanoparticles as described herein may form nanodiscs or nanospheres, depending on the absence or presence, respectively, of a filler molecule. A filler may for example be a triglyceride which is included in the core of the particle together with the payload (the nucleic acid) and the polyvalent molecule. Without being bound to theory, it is understood that including more filler will render the nanoparticles larger, up to a certain extent where the particle may become instable. Without being bound to theory, inclusion of a certain amount of filler may contribute to stabilise the nanoparticles, or it may stabilise the inclusion of the payload, or it may modulate or enhance the delivery of the nucleic acid to cells.

Therefore, in an embodiment the nanoparticle as described herein further comprises a filler molecule, preferably wherein the filler molecule is a glyceride molecule, or a cholesteryl ester, more preferably wherein the filler molecule is selected from a triglyceride and a cholesteryl ester, or combinations thereof. Preferably, triglycerides are derived from C6-C18 fatty acids, preferably C6-C12 fatty acids. Examples of cholesteryl esters are cholesteryl acetate, cholesteryl caprylate and cholesteryl oleate. Preferred filler molecules are tricaprylin, cholesteryl acetate, cholesteryl caprylate and cholesteryl oleate, more preferred is tricaprylin.

### Nanoparticle Features

The nanoparticles of the invention comprise a nucleic acid, a polyvalent molecule, a phospholipid, a sterol, a stabiliser, and optionally a filler molecule, wherein:
the amount of stabiliser, particularly a apolipoprotein, apolipoprotein derivative, apolipoprotein mimetic or apolipoprotein mimetic derivative ranges from 0.1 to 90 weight %; and/or
the amount of nucleic acid ranges from 0.01 to 90 weight %; and/or
the amount of phospholipid ranges from 0.1 to 95 weight %; and/or
the amount of sterol ranges from 0.1 to 95 weight %; and/or
the amount of polyvalent molecule ranges from 0.1 to 95 weight %,
the amount of optionally present filler comprises ranges from 0 to 95 weight %,
wherein these weight percentages are based on the combined amounts of these five components plus the optional sixth filler component, i.e. these five or six components add up to 100% of the weight of the nanoparticle.

In an embodiment, the amount of stabiliser, preferably an apolipoprotein, an apolipoprotein derivative, an apolipoprotein mimetic or an apolipoprotein mimetic derivative, ranges from 0.2 to 50 weight %, more preferably from 0.5 to 30 weight %, more preferably from 1 to 20 weight %, even more preferably from 5 to 20 weight %, such as from 5 to 15 weight %.

In an embodiment, the amount of nucleic acid ranges from 0.02 to 30 weight %, more preferably from 0.05 to 20 weight %, more preferably from 0.1 to 15 weight %, even more preferably from 0.5 to 10 weight %, such as from 0.5 to 5 weight %, from 1 to 5 weight %, or from 0.5 to 4 weight %, such as from 1.2 to 3 weight %.

In an embodiment, the amount of phospholipid ranges from 0.2 to 60 weight %, such as from 0.2 to 50 weight %, from 5 to 50 weight %, from 10 to 50 weight %, from 0.2 to 45 weight %, from 5 to 45 weight %, from 10 to 45 weight %, more preferably from 1 to 40 weight %, more preferably from 3 to 40 weight %, from 5 to 40 weight %, from 10 to 40 weight % or from 3 to 30 weight %, most preferably from 10 to 45 weight %, such as from 15 to 40 weight %.

In an embodiment, the amount of sterol ranges from 0.2 to 90 weight %, more preferably from 0.5 to 70 weight %, more preferably from 1 to 50 weight %, even more preferably from 3 to 30 weight % or from 5 to 25 weight %, such as from 8 to 20 weight %.

In an embodiment, the amount of polyvalent molecule ranges from 0.2 to 90 weight %, more preferably from 0.5 to 80 weight %, more preferably from 1 to 70 weight %, even more preferably from 1 to 30 weight %, such as from 5 to 30 weight %, from 5 to 20 weight %, from 10 to 20 weight %, from 5 to 10 weight % or from 15 to 25 weight %.

In an embodiment, the amount of optional filler molecule ranges from 0 to 90 weight %, more preferably from 0 to 80 weight %, more preferably from 0 to 70 weight %, even more preferably from 0 to 60 weight %.

The above relative amounts of the components are controlled by the ratios in which they are employed and mixed in the nanoparticle preparation protocol, as recoveries of the various components is usually quite high. By employing methods that are known in the art, the level of incorporation of various components of the nanoparticle can be assessed after the particle preparation has been concluded. Accordingly, the above ranges of incorporation of components in the nanoparticles can be assessed, either by evaluating the employed mixed-in amounts of components, or by measured values of components in the prepared nanoparticle formulation. For example, assay kits are commercially available to assess phospholipid, cholesterol, or apo A1 levels in nanoparticle samples. Furthermore, the amount of siRNA loaded and retained inside the nanoparticles can be assessed by using the RiboGreen assay.

The outer layer of the nanoparticle is composed of phospholipids, stabiliser (e.g. apolipoprotein) and sterol. In order to assemble stable nanoparticles preferably the ratio of stabiliser (e.g. apolipoprotein) to phospholipid based on weight is between 2:1 and 1:10. Therefore, in an embodiment, the employed ratio of apolipoprotein to phospholipid based on weight is between 2:1 and 1:10, more preferably between 1:1 and 1:5 even more preferably between 15:1 and 1:4 or between 2:3 and 1:4, such as 4:10.

Within the nanoparticles, the polyvalent molecule binds to the nucleic acid. The polyvalent molecule is polyvalent in positively ionizable and/or cationic groups (#N), while the nuclei acid bears multiple phosphate groups (P). The ratio between the number of N-groups (#N) and the number of P-groups (#N:P) can be varied for the nanoparticles of the invention. The N-groups (#N): P-groups (P) ratio (#N:P ratio) can be varied between 100:1 and 1:10. However, equimolar amounts or an excess of #N-groups are preferred, and #N:P ratios vary from about 50:1 to about 1:1. More preferred are #N:P ratios between about 20:1 and about 1:1. Even more preferred are #N:P ratios between about 15:1 and about 1:1.

It was found that the nanoparticles according to the invention have a relatively defined and constant size. The average size is largely determined by the core components, namely the amount and type of nucleic acid, amount of polyvalent molecule and amounts of sterol and filler molecule. It is understood that the filler is optional, and particle size can be increased by including increasing amounts of filler. In an embodiment the nanoparticles according to invention have an average diameter of about 10 to about 300 nm, preferably from about 20 to about 200 nm, more preferably from about 30 to about 100 nm.

The sizes of the nanoparticles of the invention can be assessed by methods that are known in the art. For example, dynamic light scattering (DLS) can be employed to measure the diameters of the nanoparticles. Cryo-TEM measurements can also be employed for this purpose. Both techniques may also be used to assess the distribution in diameters of prepared nanoparticle formulations. Cryo-TEM measurements can furthermore be used to assess the shape of the nanoparticles (round spheres, or other shapes). It also can assess further features of the nanoparticles (e.g. presence of outer layer or layers; presence of apolipoprotein; uniformity in nanoparticle appearance).

The nanoparticles of the invention may have a certain surface charge, that may be (slightly) negative, (slightly) positive or essentially neutral. Zeta potential analyses, or other methods that are known in the art, may be employed to assess the charged state of the nanoparticles of the invention.

The in vitro activity of the nanoparticles containing siRNA of the invention can be assessed using dual-reporter cells (e.g. RAW264.7 macrophages) transfected with Pmir-Glo plasmid that contains Firefly luciferase and Renilla luciferase gene expressing sequences. In silencing experiments, the macrophages are exposed to either nanoparticles loaded with Firefly luciferase siRNA or to nanoparticles loaded with non-specific siRNA. Next, one can assess and compare the knockdown of the luciferase activity by either of these nanoparticles. In dose dependant gene knockdown studies, one can assess the activity of the Firefly luciferase as a function of the used concentration of the Firefly luciferase siRNA. This assay reports on the potency of the employed nanoparticle formulation.

The nanoparticles as defined herein comprise a hydrophobic core and a hydrophilic surface (as a result of the incorporated phospholipid and stabiliser components), and therefore may be dissolved in water or aqueous solution such as a saline solution or buffer. The inherent properties resulting from the constituents of the nanoparticles as defined by the invention result in the nanoparticles being stable in suspension for months. Suitable aqueous buffers are known in the field, such as Phosphate Buffered Saline (PBS), Tris Buffered Saline (TBS). Suitable saline solutions are known, and non-limiting examples include aqueous solutions of NaCl or KCl. When the nanoparticle is intended to be administered to a subject, the nanoparticles should be suspended in a physiologically acceptable carrier for the purpose. For example, if the nanoparticle is intended for intravenous delivery, the physiologically acceptable carrier is typically a fluid isotonic with blood. For example, a solution of sodium chloride at 0.9% w/v concentration, a 5% w/v dextrose solution, Ringer's solution, Ringer's lactate or Ringer's acetate may be used, but other suitable carriers are known.

Therefore, in an aspect the invention relates to a composition comprising the nanoparticle according to the invention and a physiologically acceptable carrier. In an embodiment the composition is a pharmaceutical composition. It is understood that the composition may further comprise additional components, such as but not limited to pharmaceutical drugs or biopharmaceutical drugs. This may an attractive option for a combination therapy of a nucleic acid (comprised in the nanoparticle) and a drug. A drug may be a small compound, an antibody or antigen binding fragment, a further nanoparticle, etc.

It is further envisioned that the nanoparticles as described herein are used to deliver a nucleic acid therapy to a subject. Therefore, in an aspect the invention relates to the nanoparticle according to the invention, or the composition according to the invention for use as a medicament.

It is understood that the nucleic acid therapy comprising nanoparticles may be administered to a subject in need thereof. Depending on the target cells or tissue, the administration may be parenteral, e.g. intravenous, intramuscular or subcutaneous. The administration may further be oral, sublingual, topical, rectal, nasal (inhaled) or vaginal. Further the targeting of the target tissue or cells is determined by the proper choice of apolipoprotein. In an embodiment, the use of the nanoparticle or composition according to the invention, comprises delivering a nucleic acid to the myeloid compartment or the spleen. This may for example be achieved by intravenous parenteral administration. Preferably the apolipoprotein is preferably a myeloid compartment targeting apolipoprotein such as apo A1.

A further aspect provides the nanoparticle as taught herein, or the composition as taught herein for use in immunotherapy.

In an aspect the invention relates to the nanoparticle according to the invention, or the composition according to the invention for use in the treatment of a disease by stimulating or inhibiting an innate immune response, preferably wherein said disease is a disease that would benefit from stimulating or inhibiting the innate immune response in a subject, such as a disease characterized by a defective innate immune response, more preferably wherein said disease to be treated is a cancer, a cardiovascular disease, an autoimmune disorder or xenograft rejection. Therefore the nanoparticles according to the invention may be used in the treatment of any disease relating the immune system such as any immune disorder, or for the treatment of any disease or disorder where modulating the immune response is deemed a viable treatment option. By targeting the myeloid compartment, a nucleic acid therapy can successfully be delivered to progenitor cells of the different blood cell types, as opposed to already differentiated cells present in blood and tissue, such as T cells and macrophages. In doing so, the innate immune response may be modulated, e.g. stimulated or inhibited, by the nucleic acid therapy, depending on the desired result. For example, in autoimmune disorders, cardiovascular disease or xenograft rejection (prevention of), inhibition of the autoimmune response is desirable, while in cancer, stimulation of the immune response to target cancer cells is desirable.

In a further aspect the invention relates to a method for the in vivo delivery of a nucleic acid, the method comprising administering the nanoparticle according to the invention or the composition according to the invention to a subject.

In a further aspect, the invention relates to a method for treating a disease or disorder in a subject in need thereof by stimulating or inhibiting an innate immune response, the method comprising administering a therapeutically effective amount of the nanoparticle according to the invention or the composition according to the invention to the subject. In particular embodiments, the disease or disorder is a disease or disorder characterized by a defective innate immune response. In an embodiment, the disease or disorder is selected from cancer, cardiovascular disease, autoimmune disorder or xenograft rejection.

The present invention provides apolipoprotein-based nanoparticles (aNPs) with nucleic acids. Until now it was not possible to include nucleic acids in such nanoparticles as the core of such particles is hydrophobic and thus not suitable for incorporation of nucleic acids due to their hydrophilic nature. Although the use of some multivalent molecules together with nucleic acids has been described as a tool for intracellular delivery of a nucleic acid, simply combining a multivalent molecule and a nucleic acid with other lipid components does not result in the formation of the lipid nanoparticles as described herein. The present invention revolves around the realization that the nucleic acid can be incorporated in the nanoparticles by use of the polyvalent molecule described herein, and by using a two-step nanoparticle preparation process.

Therefore, in an aspect the invention relates to a method for producing a nanoparticle, comprising the step of:
a) mixing, preferably rapid mixing, of (lipid) components in organic solvent with a nucleic acid in an aqueous buffer to produce nanoparticles, wherein the (lipid) components comprise a phospholipid, a sterol, a polyvalent molecule, and optionally a filler molecule (e.g. a glyceride, for example a triglyceride); and wherein the aqueous buffer has a pH of 5.5 or lower, preferably 5.0 or lower; and
b) mixing, preferably rapid mixing, of lipid nanoparticles with a stabiliser, preferably an apolipoprotein, an apolipoprotein derivative, an apolipoprotein mimetic and/or an apolipoprotein mimetic derivative, to produce the nanoparticle at a pH between 5.5 and 9.0, preferably at a pH between 6.0 and 8.0, more preferably at a pH between 6.5 and 8.0.

Rapid mixing is known in the field and has for example been described in Hirota et al. BIOTECHNIQUES VOL. 27, NO. 2, p 286-289; Jeffs et al., Pharm Res 22, 362-372 (2005); Kulkarni et al., ACS Nano 2018, 12, 5, 4787-4795.

The aqueous buffer in step a) has a lower pH to ensure that the polyvalent molecule is positively charged, allowing binding of the nucleic acid and the polyvalent molecule within the particle. For example the buffer may have a pH of 5.5 or lower, such as 5.4, 5.3, 5.2, 5.1, or the buffer may have a pH of 5.0 or lower, such as 4.9, 4.8, 4.7, 4.6, 4.5, 4.4, 4.3, 4.2, 4.1, 4.0, 3.9, 3.8, 3.7, 3.6, 3.5 or lower. The aqueous buffer may be any buffer that does not damage the nucleic acid. An exemplary buffer is sodium acetate at pH 4.0. Next, the nanoparticle is than taken in an aqueous buffer with a pH around 6 to 8, preferably 7 to 8 more preferably around 7.4. This may for example be achieved by dialysis with an aqueous buffer in the indicated pH range. A non-limiting example of an aqueous buffer suitable for this step is 150 mM PBS at pH 7.4, but it is understood that any buffer may be used that does not damage the nucleic acid.

In step b) the nanoparticle in an aqueous buffer at pH between 6 to 8, preferably 7 to 8 is rapidly mixed with apolipoprotein in an aqueous buffer at pH between 6 to 8, preferably 7 to 8, to obtain the nanoparticles according to the invention.

In an aspect the invention relates to a nanoparticle, such as a nanoparticle according to the invention, obtained by or obtainable by the method for producing a nanoparticle as taught herein.

It is understood that nanoparticles according to the invention are able to deliver the nucleic acid in a target cell or tissue. The target cell or tissue may be in a subject, or may be in vitro or ex vivo. Therefore, in an aspect the invention relates to an in vivo, in vitro or ex vivo method for introducing a nucleic acid in a cell, the method comprising contacting the nanoparticle according to the invention or the composition according to the invention with a cell. In an embodiment the method is an in vitro or ex vivo method. In particular embodiments, the cell is a cell of the myeloid compartment or myeloid cell.

In a further aspect the invention relates to a method for the in vivo delivery of a nucleic acid, the method comprising administering the nanoparticle according to the invention or the composition according to the invention to a subject.

In a further aspect, the invention relates to a method for treating a disease or disorder in a subject in need thereof by stimulating or inhibiting an innate immune response, the method comprising administering a therapeutically effective amount of the nanoparticle according to the invention or the composition according to the invention to the subject. In an embodiment, the disease is selected from cancer, cardiovascular disease, autoimmune disorder or xenograft rejection.

The present application also provides aspects and embodiments as set forth in the following Statements:
Statement 1. A nanoparticle comprising a core and an outer layer, wherein the core comprises: —a nucleic acid;
   a polyvalent molecule;
   and wherein the outer layer comprises:
   a stabiliser;
   a phospholipid;
   a sterol; and optionally
   a filler molecule,
   wherein the polyvalent molecule has formula (I):

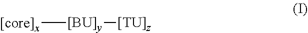

wherein the core is a nitrogen or is a C₁-C₁₈ linear, branched or cyclic group that contains 1 to 15 nitrogen heteroatoms and that optionally contains 1 to 4 oxygen heteroatoms; and
wherein x represents the number of connections from the core to the branching units BU (or to the terminal units TU, when y=0), where x is 1, 2, 3, 4, 5, 6, 7, 8, 9, 10 or 12, and where these connections are all made from nitrogen atoms of the core to carbon atoms of the branching unit BU (or of the terminal unit TU); and
wherein the branching unit BU has formula (IIa), (IIb), (IIc) or (IId):

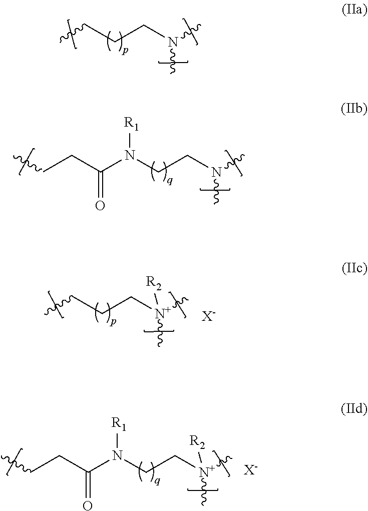

wherein (IIa) represents an n-alkylene-nitrogen spacer that is connected at the CH₂-end to the core or to a BU that is closer to the core, and that is connected at the N-end to two terminal units (TUs) or to two branching units (BUs) that are closer to the TUs, where the connections are given in wavy bonds, and wherein p is 1, 2, 3 or 4; and
wherein (IIb) represents an amide containing n-alkylene-nitrogen spacer that is connected at the CH₂-end to the core or to a BU that is closer to the core, and that is connected at the N-end to two terminal units (TUs) or to two branching units (BUs) that are closer to the TUs, where the connections are given in wavy bonds, wherein q is 1, 2, 3, 4 or 5, and wherein R₁ is a hydrogen, a methyl, an ethyl, an n-propyl or an iso-propyl group; and
wherein (IIc) and (IId) are defined as (IIa) and (IIb), respectively, and wherein R₂ is a methyl, ethyl, n-propyl, benzyl, acetamide (—CH₂—C(O)NH₂) or 2-hydroxy-ethylene group, and wherein X⁻ is the counter anion to the quaternary amine cation moiety; and
wherein y represents the specific and discrete generation number of the polyvalent molecule, where this number indicates how many successive BU layers are incorporated in the polyvalent molecule, as counted from traveling from the inner core to the outer TU groups, and where y is 0, 1, 2, 3, 4 or 5; and
wherein for each separate generational layer the BU can independently be selected from formulas (IIa), (IIb), (IIc) or (IId), i.e. the first BU emanating from the core, representing the first generational layer BU, can be different from the second BU emanating further away from the core (representing the second generational layer BU), and this one may be different from the third, the fourth and the fifth BU emanating further away from the core; and
wherein the terminal units (TU) are individually and independently chosen from a hydrogen and a C₁-C₃₀ alkyl, aryl, arylene-alkyl or alkylene-aryl group that optionally contains 1 to 8 heteroatoms that are individually and independently selected from the group consisting of O and N, with the proviso that not all TUs in the polyvalent molecule are hydrogens; and
wherein z represents the total number of TU groups that are attached to the polyvalent molecule, where z is 1 to 128; and
wherein the polyvalent molecule as according to formula (I) is polyvalent in positively ionizable and/or cationic groups, where N represents the cumulative number of positively ionizable and cationic groups in the polyvalent molecule, and where N is 2 or higher.
Statement 2. The nanoparticle according to statement 1, wherein the polyvalent molecule has formula (I):

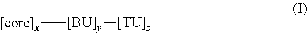

wherein y=0, 1, 2, or 3.
Statement 3. The nanoparticle according to any one of the preceding statements, wherein the polyvalent molecule is a first, second or third generation dendrimer selected from a poly(propylene imine) (PPI) dendrimer or a polyamidoamine (PAMAM) dendrimer, most preferably a PPI dendrimer, or a modification thereof.
Statement 4. The nanoparticle according to any of the preceding statements, wherein the polyvalent molecule has a structure selected from:

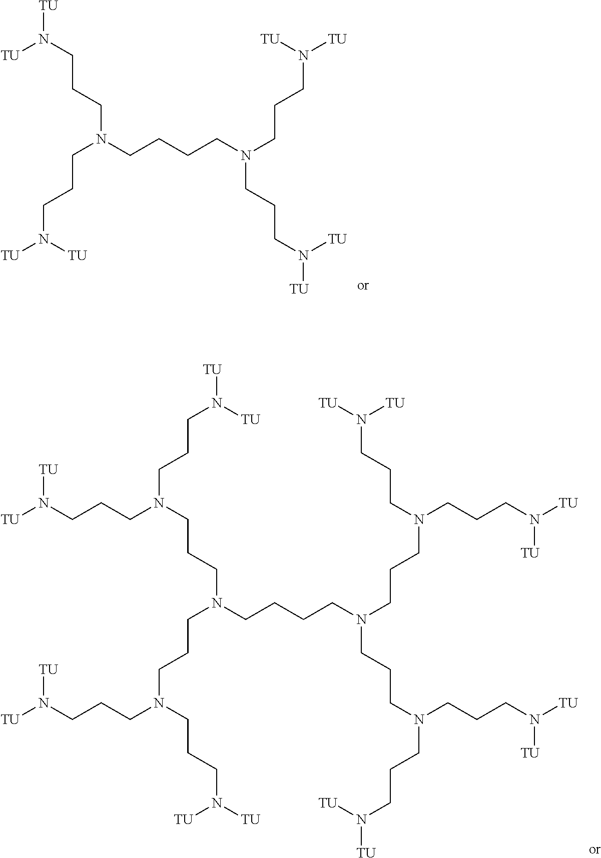

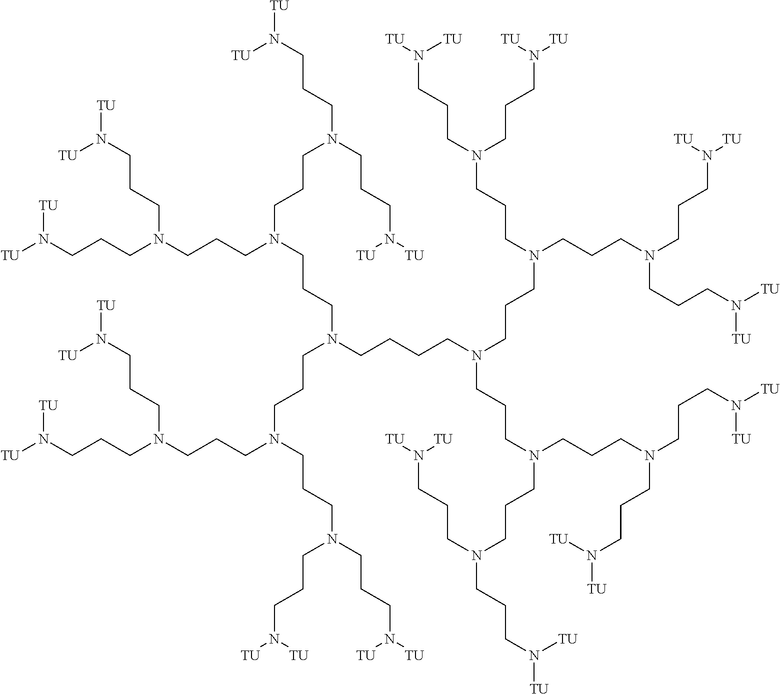

wherein the terminal units (TU) are individually and independently chosen from a hydrogen and a C₁-C₃₀ alkyl, aryl, arylene-alkyl or alkylene-aryl group that optionally contains 1 to 8 heteroatoms that are individually and independently selected from the group consisting of O and N, with the proviso that not all TUs in the polyvalent molecule are hydrogens.
Statement 5. The nanoparticle according to any one of the preceding statements, wherein the stabiliser is:
   an apolipoprotein, an apolipoprotein derivative, an apolipoprotein mimetic and/or an apolipoprotein mimetic derivative;
   a polyethylene glycol or polyethylene glycol modified molecule;
   a hydrophilic polymer;
   a polysaccharide or polysaccharide modified molecule; or
   a polysarcosine or polysarcosine modified molecule.
Statement 6. The nanoparticle according to any one of the preceding statements, wherein the apolipoprotein, apolipoprotein derivative, apolipoprotein mimetic or apolipoprotein mimetic derivative is selected form apo A1, apo A1-Milano, apo A2, apo A4, apo A5, apo B48, apo B100, apo C-I, apo C-II, apo C-III, apo C-IV, apo D, apo E, apo F, apo H, apo L and apo M or a mimetic or derivatives thereof,
preferably selected from apo A1, apo A2, apo A4, apo A5, apo B100, apo C-I, apo C-II, apo C-III, apo C-IV and apo E or a mimetic or derivatives thereof,
more preferably selected from apo A1, apo A4, apo A5, apo B100, apo C-III and apo E or a mimetic or derivatives thereof,
most preferably selected from apo A1, apo B100 and apo E or a mimetic or derivatives thereof.
Statement 7. The nanoparticle according to any one of the preceding statements, wherein the nucleic acid is RNA, DNA or a nucleic acid analogue,
   preferably wherein the RNA is microRNA (miRNA), small interfering RNA (siRNA), piwi-interacting RNA (piRNA), small nuclear RNA (snoRNA), transfer RNA (tRNA), tRNA-derived small RNA (tsRNA), small regulatory RNA (srRNA), messenger RNA (mRNA), modified mRNA, ribosomal RNA (rRNA), self-amplifying RNA (saRNA), circular RNA (circRNA), long non-coding RNA (lncRNA), or guide RNA (gRNA) or combinations thereof and/or modifications thereof; or
   preferably wherein the DNA is single stranded or double stranded DNA; or
   preferably wherein the nucleic acid is an antisense oligonucleotide which is single stranded DNA or RNA consisting or comprising of nucleotide or nucleoside analogues containing modifications of the phosphodiester backbone or the 2′ ribose, more preferably wherein the nucleotide or nucleoside analogues are selected from locked nucleic acid (LNA), bridged nucleic acid (BNA), morpholino or peptide nucleic acid (PNA).
Statement 8. The nanoparticle according to any one of the preceding statements, wherein the sterol is preferably selected from sterol, cholesterol, ergosterol, hopanoids, hydroxysteroid, phytosterol, steroids, zoosterol, stigmasterol, or β-sitosterol, or combinations thereof.
Statement 9. The nanoparticle according to any one of the preceding statements, wherein:
the phospholipid is selected from a phosphatidylcholine, a phosphatidylethanolamine, a phosphatidylserine and a phosphatidylglycerol or combinations thereof, preferably wherein at least one, more preferably both, of the acyl groups in the phospholipid are derived from long chain fatty acids, even more preferably wherein said long chain fatty acids are selected from lauric acid, lauroleic acid, myristic acid, myristoleic acid, palmitic acid, palmitoleic acid, stearic acid, oleic acid and linoleic acid, or combinations thereof.
Statement 10. The nanoparticle according to any one of the preceding statements, further comprising a filler molecule, preferably wherein the filler molecule is a glyceride molecule, more preferably wherein the filler molecule is selected from a triglyceride, a modified triglyceride, and a cholesteryl ester, or combinations thereof, preferably wherein the triglyceride is derived from C6-C18 fatty acids, preferably tricaprylin and/or wherein the cholesteryl ester is cholesteryl acetate, cholesteryl caprylate and/or cholesteryl oleate.
Statement 11. The nanoparticle according to any one of the preceding statements, wherein:
the amount of apolipoprotein, apolipoprotein derivative, apolipoprotein mimetic or apolipoprotein mimetic derivative ranges from 0.1 to 90 weight %; and/or
the amount of nucleic acid ranges from 0.01 to 90 weight %;
the amount of phospholipid ranges from 0.1 to 95 weight %; and/or
the amount of sterol ranges from 0.1 to 95 weight %; and/or
the amount of polyvalent molecule ranges from 0.1 to 95 weight %,
the amount of optionally present filler comprises ranges from 0 to 95 weight %,
wherein the weight percentages are based on the combined amounts of these five components plus the optional sixth filler component, i.e. these five or six components add up to 100% of the weight of the nanoparticle.
Statement 12. A composition comprising the nanoparticle according to any one of the preceding statements and a physiologically acceptable carrier, preferably wherein the composition is a pharmaceutical composition.
Statement 13. The nanoparticle according to any one of statements 1 to 11, or the composition according to statement 12 for use as a medicament.
Statement 14. The nanoparticle or composition for use according to statement 13, the use comprising delivering a nucleic acid to the myeloid compartment or the spleen.
Statement 15. Method for producing a nanoparticle, comprising the step of:
   a) mixing, preferably rapid mixing, of (lipid) components in organic solvent with a nucleic acid in an aqueous buffer to produce nanoparticles, wherein the (lipid) components comprise a phospholipid, a sterol, a polyvalent molecule, and optionally a filler molecule; and wherein the aqueous buffer has a pH of 5.5 or lower, preferably 5.0 or lower; and
   b) mixing, preferably rapid mixing, of lipid nanoparticles with a stabiliser, preferably an apolipoprotein, an apolipoprotein derivative, an apolipoprotein mimetic and/or an apolipoprotein mimetic derivative, to produce the nanoparticle at a pH between 5.5 and 9.0, preferably at a pH between 6.0 and 8.0, more preferably at a pH between 6.5 and 8.0.
   All cited references are herewith incorporated by reference in their entirety.

### EXAMPLES

### Example 1—Design and Synthesis of Polyvalent Molecules

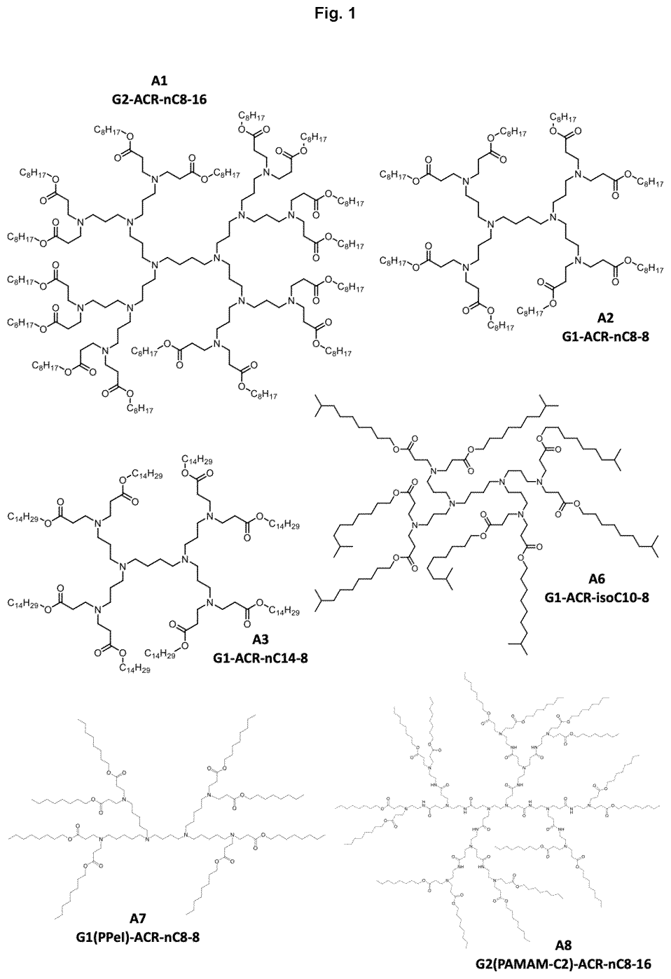

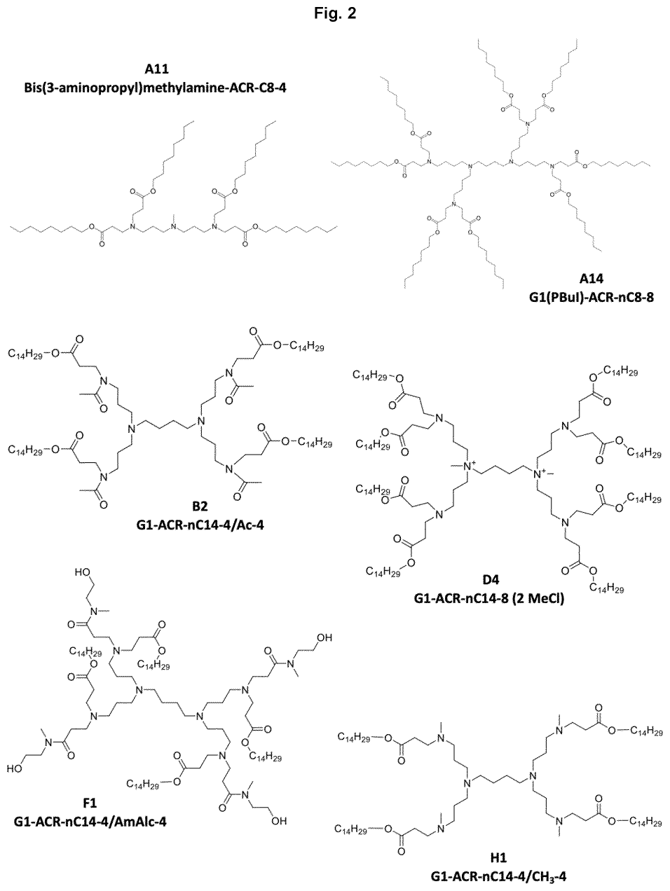

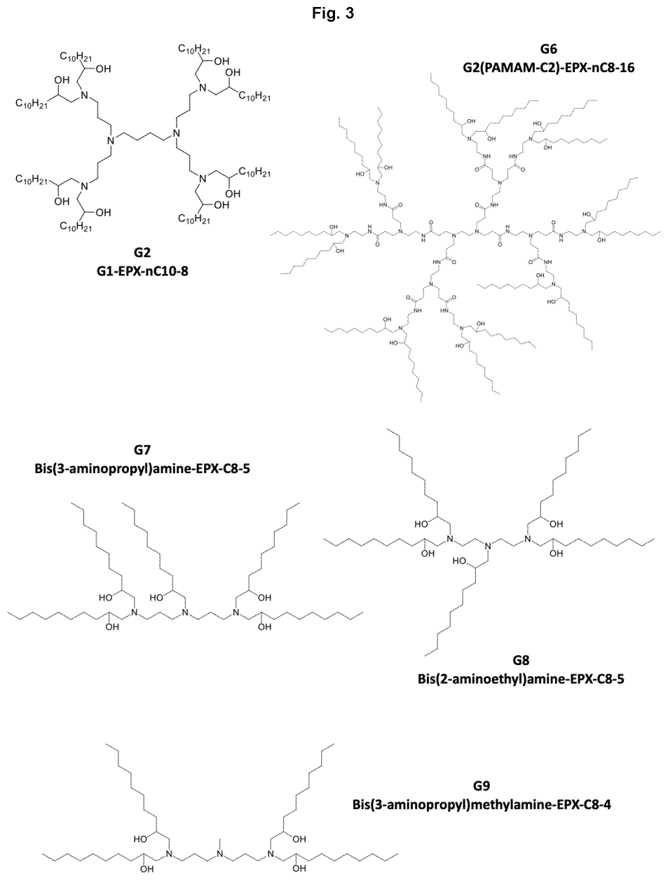

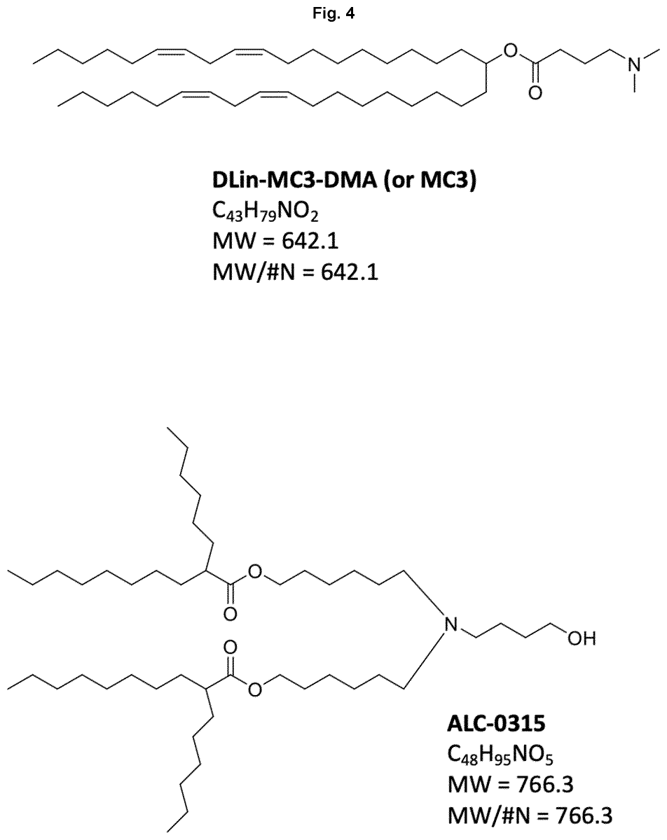

In Table S1 and S2 an overview of features of the prepared polyvalent molecules is provided. FIGS. 1, 2, and 3 provide molecular structures of a series of polyvalent molecules.

TABLE S1
Polyvalent molecule features.

| | Polyvalent | MW | #N | MW/#N | | | | |
|---|---|---|---|---|---|---|---|---|
| Entry | material | (Dalton) | (—) | (Dalton) | BU | x | y | z |
| 1 | A1 | 3722 | 14 | 266 | IIa | 4 | 2 | 16 |
| 2 | A2 | 1791 | 6 | 298 | IIa | 4 | 1 | 8 |
| 3 | A3 | 2464 | 6 | 411 | IIa | 4 | 1 | 8 |
| 4 | A4 | 1342 | 6 | 224 | IIa | 4 | 1 | 8 |
| 5 | A5 | 5068 | 14 | 362 | IIa | 4 | 2 | 16 |
| 6 | A6 | 2015 | 6 | 336 | IIa | 4 | 1 | 8 |
| 7 | A7 | 1903 | 6 | 317 | IIa (p = 3) | 4 | 1 | 8 |
| 8 | A8 | 4378 | 14 | 313 | IIb | 4 | 2 | 16 |
| 9 | A9 | 1053 | 3 | 351 | None | 5 | 0 | 5 |
| 10 | A10 | 1025 | 3 | 342 | None | 5 | 0 | 5 |
| 11 | A11 | 882 | 3 | 294 | None | 4 | 0 | 4 |
| 12 | A12 | 1053 | 3 | 351 | None | 5 | 0 | 5 |
| 13 | A13 | 1791 | 6 | 298 | IIa | 4 | 1 | 8 |
| 14 | A14 | 1847 | 6 | 308 | IIa (p = 2) | 4 | 1 | 8 |
| 15 | A15 | 1990 | 6 | 332 | IIb | 4 | 1 | 8 |
| 16 | B1 | 1326 | 2 | 663 | IIa | 4 | 1 | 8 |
| 17 | B2 | 1559 | 2 | 779 | IIa | 4 | 1 | 8 |
| 18 | B3 | 2792 | 6 | 465 | IIa | 4 | 2 | 16 |
| 19 | B4 | 3257 | 6 | 543 | IIa | 4 | 2 | 16 |
| 20 | C1 | 7359 | 30 | 245 | IIa | 4 | 3 | 32 |
| 21 | C2 | 1735 | 6 | 289 | IIa | 4 | 1 | 8 |
| 22 | D1 | 717 | 2 | 359 | IIa | 4 | 1 | 8 |
| 23 | D2 | 818 | 2 | 409 | IIc (MeCl) | 4 | 1 | 8 |
| 24 | D3 | 418 | 6 | 70 | IIc (MeCl) | 4 | 1 | 8 |
| 25 | D4 | 2565 | 6 | 428 | IIc (MeCl) | 4 | 1 | 8 |
| 26 | D5 | 1574 | 6 | 262 | IIa | 4 | 2 | 16 |
| 27 | D6 | 1877 | 6 | 313 | IIc (MeCl) | 4 | 2 | 16 |
| 28 | D7 | 1368 | 14 | 98 | IIc (MeCl) | 4 | 2 | 16 |

MW = molecular weight; #N = number of ionizable and/or cationic groups in the material; BU = branching unit as defined by Formula (II); x, y and z as according to Formula (I). All (IIa) are with p = 1, except where indicated. All (IIb) and (IIc) are with q = 1 and p = 1, respectively. (IIc) are methylated dendrimers either with chloride or iodide counter anions, as indicated with MeCl or Mel.

TABLE S2
Polyvalent molecule features.

| | Polyvalent | MW | #N | MW/#N | | | | |
|---|---|---|---|---|---|---|---|---|
| Entry | material | (Dalton) | (—) | (Dalton) | BU | X | Y | z |
| 29 | E1 | 1350 | 6 | 225 | IIa | 4 | 1 | 8 |
| 30 | E2 | 1115 | 3 | 372 | None | 4 | 0 | 4 |
| 31 | E3 | 1343 | 3 | 448 | None | 5 | 0 | 5 |
| 32 | E4 | 1072 | 3 | 357 | None | 5 | 0 | 5 |
| 33 | E5 | 1057 | 2 | 529 | None | 4 | 0 | 4 |
| 34 | F1 | 1907 | 6 | 318 | IIa | 4 | 1 | 8 |
| 35 | G1 | 1567 | 6 | 261 | IIa | 4 | 1 | 8 |
| 36 | G2 | 1791 | 6 | 299 | IIa | 4 | 1 | 8 |
| 37 | G3 | 3722 | 14 | 266 | IIa | 4 | 2 | 16 |
| 38 | G4 | 7585 | 30 | 253 | IIa | 4 | 3 | 32 |
| 39 | G5 | 1903 | 6 | 317 | IIa (p = 3) | 4 | 1 | 8 |
| 40 | G6 | 3930 | 14 | 281 | IIb | 4 | 2 | 16 |
| 41 | G7 | 913 | 3 | 304 | None | 5 | 0 | 5 |
| 42 | G8 | 885 | 3 | 295 | None | 5 | 0 | 5 |
| 43 | G9 | 770 | 3 | 257 | None | 4 | 0 | 4 |
| 44 | H1 | 1446 | 6 | 241 | IIa | 4 | 1 | 8 |
| 45 | J1 | 1270 | 2 | 635 | IIa | 4 | 1 | 8 |
| 46 | J2 | 1554 | 2 | 777 | IIc (Mel) | 4 | 1 | 8 |
| 47 | J3 | 1270 | 2 | 635 | IIc (MeCl) | 4 | 1 | 8 |
| 48 | J4 | 5502 | 14 | 393 | IIa | 4 | 3 | 32 |
| 49 | J5 | 7489 | 14 | 535 | IIc (Mel) | 4 | 3 | 32 |
| 50 | J6 | 6209 | 14 | 443 | IIc (MeCl) | 4 | 3 | 32 |
| 51 | J7 | 821 | 2 | 411 | IIa | 4 | 1 | 8 |
| 52 | J8 | 1022 | 2 | 511 | IIb | 4 | 1 | 8 |
| 53 | J9 | 1887 | 6 | 315 | IIa | 4 | 1 | 8 |
| 54 | J10 | 1887 | 6 | 315 | IIa | 4 | 1 | 8 |
| 55 | J11 | 1999 | 6 | 333 | IIa | 4 | 1 | 8 |

MW = molecular weight; #N = number of ionizable and/or cationic groups in the material; BU = branching unit as defined by Formula (II); X, Y and z as according to Formula (I). All (IIa) are with p = 1, except where indicated. All (IIb) and (IIc) are with q = 1 and p = 1, respectively. (IIc) are methylated dendrimers either with chloride or iodide counter anions, as indicated with MeCl or Mel.

### Materials and Methods

All reagents, chemicals, materials and solvents were obtained from commercial sources and were used as received. All solvents were of AR quality. DCM=dichloromethane; CHCl₃=chloroform; MeCN=acetonitrile; MeOH is methanol; EtOH=ethanol; IPA=iso-propanol; DMSO=dimethyl-sulfoxide; THF=tetrahydrofuran; 1,1,1,3,3,3-hexafluoroisopropanol=HFIP; DMF=dimethylformamide. In the synthetic procedures, equivalents (eq) are molar equivalents.

NMR spectra were recorded on a Bruker Avance III HD spectrometer at 298 K (400 MHz and 100 MHz for ¹H-NMR and ¹³C-NMR, respectively). Chemical shifts are reported in ppm downfield from TMS at room temperature. Abbreviations used for splitting patterns are s=singlet, t=triplet, q=quartet, m=multiplet and br=broad.

HPLC-PDA/ESI-MS was performed using a Shimadzu LC-10 AD VP series HPLC coupled to a photodiode array detector (Finnigan Surveyor PDA Plus detector, Thermo Electron Corporation) and an ion-trap mass spectrometer (LCQ Fleet, Thermo Scientific), applying electrospray ionization (ESI). HPLC-analyses were performed using a Alltech Alltima HP C18 3μ column using an injection volume of 1-4 μL, a flow rate of 0.2 mL min-1 and typically a gradient (5% to 100% in 10 min, held at 100% for a further 3 min) of MeCN in H₂O (both containing 0.1% formic acid) at 298 K.

MALDI-TOF-MS was measured on an Autoflex Speed (Bruker) spectrometer using α-cyano-4-hydroxycinnamic acid (CHCA), 2,5-dihydroxybenzoic acid (DHB) or trans-2-[3-(4-tert-butylphenyl)-2-methyl-2-propenylidene]malononitrile (DCTB) matrices.

### Building Blocks

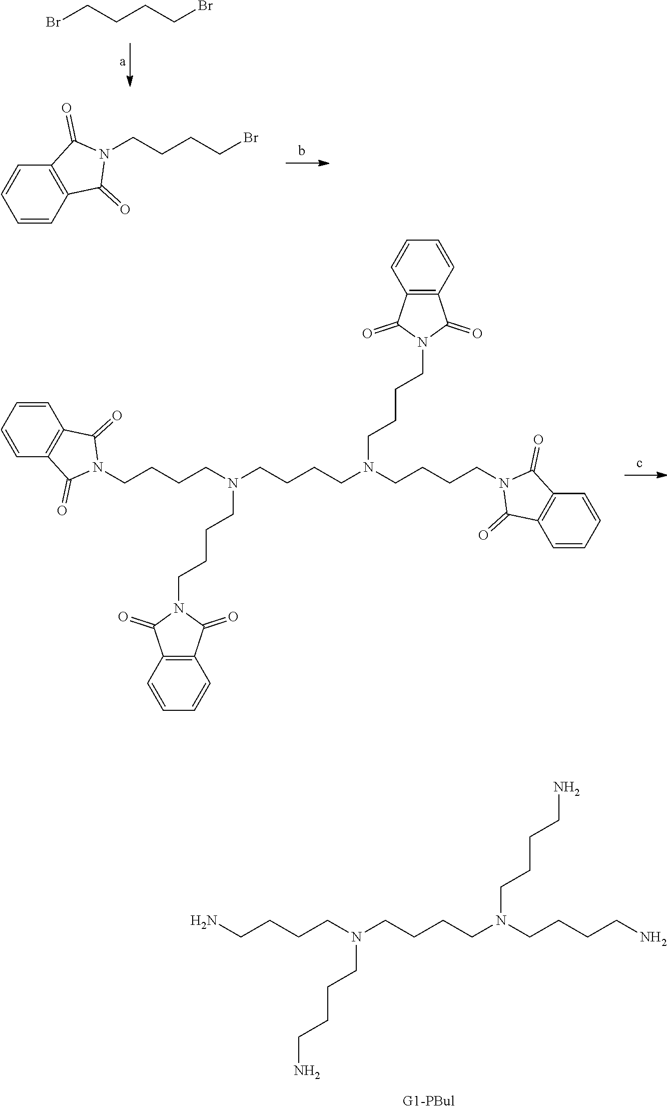 [SMILES: BrCCCCBr ; O=C1c2ccccc2C(=O)N1CCCCBr ; O=C1c2ccccc2C(=O)N1CCCCN(CCCCN(CCCCN1C(=O)c2ccccc2C1=O)CCCCN1C(=O)c2ccccc2C1=O)CCCCN1C(=O)c2ccccc2C1=O ; NCCCCN(CCCCN)CCCCN(CCCCN)CCCCN]

Synthesis of G1 poly(butylene imine) dendrimer. (a) Potassium phthalimide, DMF, 48h, rt; (b) 1,4-diaminobutane, K₂CO₃, MeCN, 40h, reflux; (c) N₂H₄·H₂O, EtOH, 16h, reflux.

### N-(4-Bromobutyl)phthalimide

1,4-Dibromobutane (86.4 g, 0.4 mol, 4 eq) and potassium phthalimide (18.5 g, 0.1 mol, 1 eq) were dissolved in DMF (75 mL). (Potassium phthalimide was not completely dissolved). The reaction mixture was stirred for 48 h at room temperature. The reaction mixture was then poured into water (600 mL), transferred to an extraction funnel and extracted with diethyl ether (2×50 mL). The combined ether layers were washed with a saturated KCl-solution (2×25 mL). The ether layer was dried using Na₂SO₄, the suspension was filtered, and the filtrate was concentrated in vacuo. The product was purified using silica column chromatography using mixtures of heptane/DCM (0-100% DCM) yielding a white solid. The last impurities were removed by stirring the solid in n-pentane for 16 h at room temperature, filtration, washing and drying of the residue in vacuo yielding the product (14.9 g 52.7 mmol, 53%). Alternatively the crude product could be purified via recrystallisation from n-heptane.

¹H NMR (400 MHz, Chloroform-d) δ 7.85 (m, 2H), 7.72 (m, 2H), 3.73 (t, J=6.6 Hz, 2H), 3.45 (t, J=6.3 Hz, 2H), 1.97-1.80 (m, 4H).

### N,N,N′,N′-Tetra(4-phthalimidobutyl)-1,4-diaminobutane

N-(4-Bromobutyl)phthalimide (1.41 g, 5 mmol, 5 eq), 1,4-diaminobutane (88 mg, 1 mmol, 1 eq) and K₂CO₃ (691 mg, 5 mmol, 5 eq) were mixed together in MeCN (5 mL) and stirred for 40 h at reflux. The reaction mixture was cooled down, the suspension was filtered, and the filtrate was concentrated in vacuo. The product was purified using silica column chromatography eluting with mixtures of MeOH/CHCl₃ (0-10% MeOH) to give the product as an amber viscous oil (222 mg, 0.25 mmol, 25%).

¹H NMR (399 MHz, Chloroform-d) δ 7.86-7.77 (m, 7H), 7.75-7.64 (m, 8H), 3.68 (t, J=7.0 Hz, 8H), 2.69 (sb, 4H), 2.39 (s, 8H), 1.68 (s, 13H), 1.40 (d, J=45.6 Hz, 10H). ¹³C NMR (100 MHz, CDCl₃) δ 168.19, 133.73, 132.03, 123.00, 53.77, 53.25, 37.73, 26.42, 24.74, 24.25. ESI-MS: m/z Calc. for C₅₂H₅₆N₆O₈ 892.42; Obs. 893.58 [M+H]⁺ 447.42, [M+H]²⁺.

### N,N,N′,N′-Tetra(4-aminobutyl)-1,4-diaminobutane (G1-PBuI)

Hydrazine hydrate (0.96 mL, 19.8 mmol, 20 eq) was added to a stirring suspension of N,N,N′,N′-tetra(4-phthalimidobutyl)-1,4-diaminobutane (884 mg, 0.99 mmol, 1 eq) in EtOH (30 mL). The reaction mixture was heated to reflux for 16h giving a white suspension. The suspension was filtered over a glass fritted funnel and the residue was washed with ethanol. The filtrate was concentrated in vacuo giving a white sticky material (100 mg, 0.27 mmol, 27%) which was a mixture of product and 2,3-dihydrophthalazine-1,4-dione.

¹H NMR (399 MHz, Chloroform-d) δ 2.75-2.65 (m, 8H), 2.46-2.34 (m, 12H), 2.24 (s br, 8H), 1.56-1.42 (m, J=4.4 Hz, 16H), 1.42-1.32 (m, 4H). ESI-MS: m/z Calc. for C₂₀H₄₈N₆ 372.39; Obs. 373.50 [M+H]+.

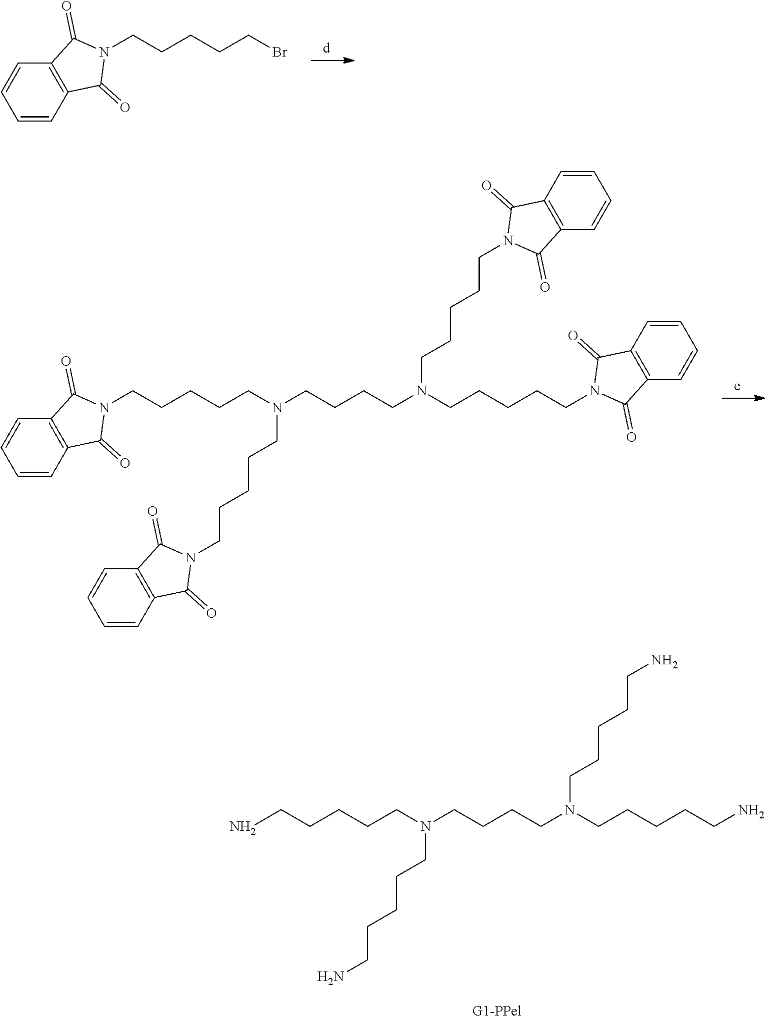 [SMILES: O=C1c2ccccc2C(=O)N1CCCCCBr ; O=C1c2ccccc2C(=O)N1CCCCCN(CCCCCN1C(=O)c2ccccc2C1=O)CCCCN(CCCCCN1C(=O)c2ccccc2C1=O)CCCCCN1C(=O)c2ccccc2C1=O ; NCCCCCN(CCCCCN)CCCCN(CCCCCN)CCCCCN]

Synthesis of G1 poly(pentylene imine) dendrimer. (d) 1,4-diaminobutane, K₂CO₃, MeCN, 40h, reflux; (e) N₂H₄·H₂O, EtOH, 16h, reflux; (f) propan-2-ol, 60° C., 112h.

### N,N,N′,N′-Tetra(5-phthalimidopentyl)-1,4-diaminobutane

N-(5-Bromopentyl)phthalimide (14.8 g, 50 mmol, 5 eq), 1,4-diaminobutane (882 mg, 10 mmol, 1 eq) and K₂CO₃ (12 mg, 80 mmol, 8 eq) were mixed in MeCN (50 mL) and were stirred for 40 h at reflux. The reaction mixture was cooled down, the suspension was filtered, and the filtrate was concentrated in vacuo. The product was purified using silica column chromatography eluting with mixtures of MeOH/CHCl₃ (0-10% MeOH) to give an amber very viscous oily product (3.12 g, 3.28 mmol, 33%).

¹H NMR (400 MHz, Chloroform-d) δ 7.82 (dd, J=5.4, 3.0 Hz, 8H), 7.74-7.65 (m, 8H), 3.67 (t, J=7.2 Hz, 8H), 2.41 (m, 12H), 1.68 (p, J=7.4 Hz, 8H), 1.48 (m, 8H), 1.39 (m, 4H), 1.37-1.20 (m, 8H). ¹³C NMR (101 MHz, CDCl₃) δ 168.29, 133.81, 132.07, 123.07, 53.62, 53.50, 37.77, 28.36, 25.89, 24.62, 24.24. ESI-MS: m/z Calc. for C₅₆H₆₄N₆O₈ 948.48; Obs. 949.58 [M+H]⁺ 475.50, [M+H]²⁺.

### N,N,N′,N′-Tetra(5-aminopentyl)-1,4-diaminobutane (G1-PPeI)

Hydrazine hydrate (3.08 g, 61.5 mmol, 20 eq) was added to a stirring suspension of N,N,N′,N′-tetra(5-phthalimidopentyl)-1,4-diaminobutane (2.92 g, 3.07 mmol, 1 eq) in EtOH (50 mL). The reaction mixture was heated to reflux for 16h giving a white suspension. The suspension was filtered over a glass fritted funnel and washed with ethanol. The filtrate was concentrated in vacuo and redissolved in chloroform giving a suspension. The suspension was filtered and the filtrate was concentrated in vacuo. To remove the last traces of 2,3-dihydrophthalazine-1,4-dione, the residue was redissolved in a mixture of n-pentane/chloroform 1:1, filtered and the filtrate was concentrated in vacuo giving the product as a light yellow viscous oil (950 mg, 2.22 mmol, 72%).

¹H NMR (400 MHz, Chloroform-d) δ 2.69 (t, J=7.0 Hz, 8H), 2.39 (m, 12H), 1.67 (s br, 8H), 1.45 (m, 20H), 1.37-1.26 (m, 8H). ESI-MS: m/z Calc. for C₂₄H₅₆N₆ 428.46; Obs. 429.58 [M+H]+.

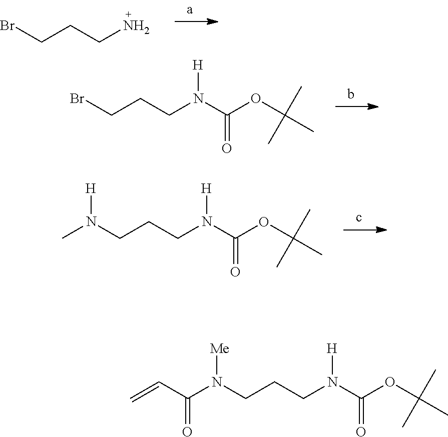

Synthesis of tert-butyl (3-(N-methylacrylamido)propyl)carbamate. (a) (Boc)₂O, DCM, 0° C. to r.t., 72h, 95%; (b) 33 w/w % methylamine in MeOH, KI, EtOH, 50° C., 20 h, 63%; (c) acryloyl chloride, TEA, DCM, 0° to r.t., 16 h, 68%.

### tert-Butyl (3-bromopropyl)carbamate

To an ice cooled solution of 3-bromopropylamine hydrobromide salt (8.6 g, 39.3, 1 eq) in MeOH (40 mL) was added triethylamine (4.2 g, 41.3 mmol, 1.05 eq). Then di-tert-butyl carbonate was added portion wise to the reaction mixture. The temperature of the reaction mixture was slowly raised to room temperature and stirred for another 16 h. The mixture then was concentrated in vacuo, re-dissolved in a mixture of dichloromethane and water, transferred to a separation funnel and the water layer was extracted with dichloromethane (3×). The collected dichloromethane layers were washed with a 0.5M citric acid solution (1×25 mL), and with brine (1×25 mL). The dichloromethane layers were dried using Na₂SO₄ and the suspension was filtered. Evaporation of the solvent in vacuo yielded the product as a colorless oil (8.86 g, 37.2 mmol, 95%).

### tert-Butyl (3-(methylamino)propyl)carbamate

To a stirring solution of tert-butyl (3-bromopropyl)carbamate (8.86 g, 37.2 mmol, 1 eq) in ethanol (30 mL) was added a 40% w/w solution of methylamine in MeOH (45 mL, 446 mmol, 10 eq) and KI (0.74 g, 4.46 mmol, 0.1 eq) and the temperature was raised to 50° C. After 20 h at 50° C., the reaction mixture was concentrated in vacuo. The residue was re-dissolved in DCM and water and the water layer was extracted with DCM (3×25 mL). The collected DCM-layers were dried using Na₂SO₄, the suspension was filtered and the filtrate was concentrated in vacuo giving a colorless oily by-product. The water layer was basified with 6M NaOH-solution to pH>10 and extracted with CHCl₃ (3×50 mL). The collected CHCl₃-layers were dried using Na₂SO₄ and the suspension was filtered. The filtrate was concentrated in vacuo to yield a colorless oil (4.84 g, 25.7 mmol, 58%).

¹H NMR (399 MHz, Chloroform-d) δ 5.06 (s, 1H), 3.20 (q, J=6.3 Hz, 2H), 2.63 (t, J=6.7 Hz, 2H), 2.42 (s, 3H), 1.65 (p, J=6.7 Hz, 2H), 1.44 (s, 11H), 1.34 (s, 2H). ESI-MS: m/z Calc. for C₉H₂₀N₂O₂ 188.15. Measured [M+H]⁺ 189.17.

### tert-Butyl (3-(N-methylacrylamido)propyl)carbamate

A solution of acryloyl chloride (2.36 g, 26.1 mmol, 1.1 eq) in DCM (10 mL) was added dropwise to an ice cooled solution of tert-butyl (3-(methylamino)propyl)carbamate (4.43 g, 23.5 mmol, 1 eq) and triethylamine (3.9 mL, 28.2 mmol, 1.2 eq) in DCM (50 mL). After addition the reaction temperature was allowed to raise to room temperature. The reaction mixture was stirred for 20 h at room temperature. The reaction mixture was transferred to a extraction funnel and the DCM-layer was washed with water (1×25 mL), 1M KHSO₄-solution (1×25 mL), water (1×25 mL), 1 M NaHCO₃-solution and water (1×25 mL). The DCM-layer was dried using Na₂SO₄, the suspension was filtered and the filtrate was concentrated in vacuo yielding crude product as a yellow oil. The acryl-amide product was further purified using column chromatography eluting with mixtures of MeOH and DCM (0 to 5% MeOH in DCM) yielding a slightly yellow oil (3.85 g, 15.9 mmol, 68%).

¹H NMR (399 MHz, Chloroform-d) δ 6.57 (m, 1H), 6.32 (dd, J=16.7, 2.0 Hz, 1H), 5.70 (m, 1H), 5.42 (s, 0.74H), 4.63 (s, 0.26H), 3.51 (t, J=6.4 Hz, 1.42H), 3.41 (t, J=7.7 Hz, 0.65H), 3.11 (m, 4H), 3.06 (s, 2.33H), 3.00 (s, 0.87H), 1.80 (p, J=7.2 Hz, 0.57H), 1.71 (p, J=6.3 Hz, 1.47H), 1.44 (s, 9H). ESI-MS: m/z Calc. for C₁₂H₂₂N₂O₃ 242.16. Measured [M+Na]+265.17.

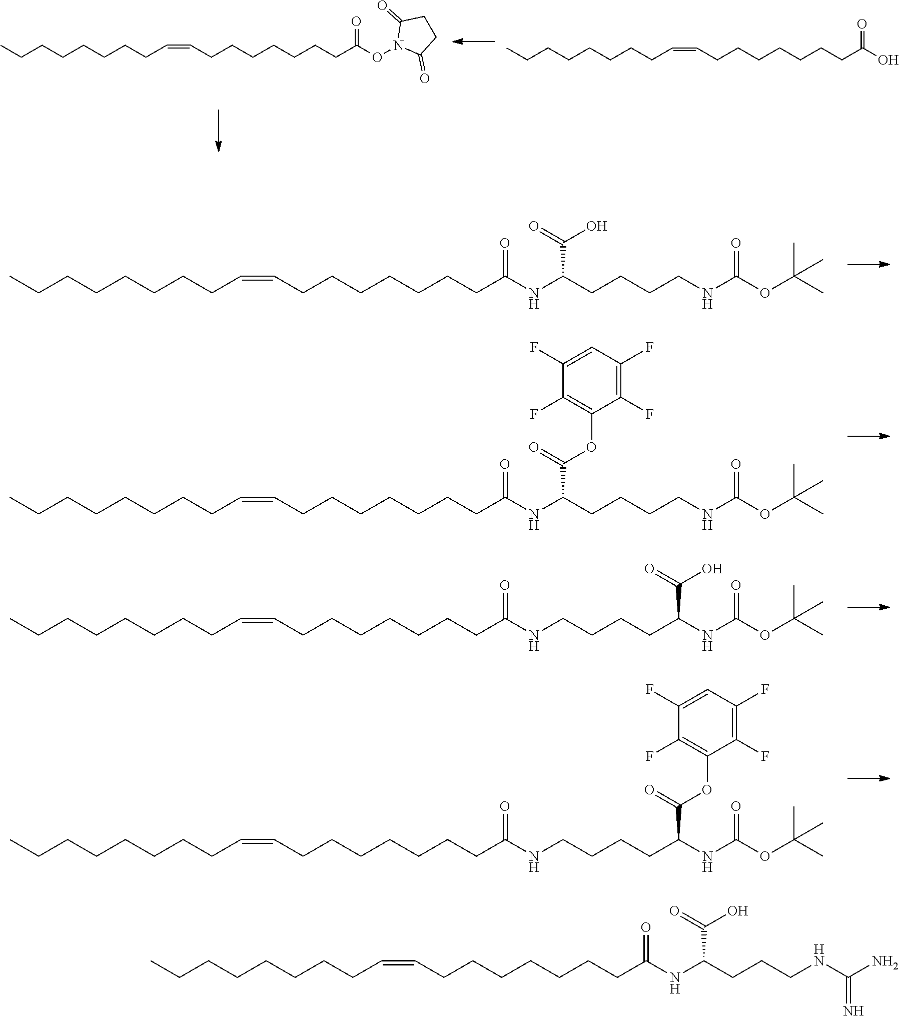

### The Synthesis of Oleyl-Amino-Acid Building Blocks

### 2,5-Dioxopyrrolidin-1-yl oleate (oleyl-NHS ester)

N-Hydroxy-succinimide (3.37 g, 29.3 mmol, 1.14 eq) and DIPEA (8.0 mL) were dissolved in THF (60 mL) in a nitrogen atmosphere. A solution of oleoyl chloride (7.74 g, 25.7 mmol, 1 eq) in THF (50 mL) was dropwise added at 0° C. After 20 h at room temperature the reaction mixture was evaporated till dryness. The crude product was dissolved in chloroform (200 mL) and washed with aqueous 0.2M NaOH (500 mL). The water layer was extracted with chloroform (100 mL). The combined chloroform fractions were washed with brine (200 mL). Purification by column chromatography on silica (elution by chloroform:ethyl acetate 80:20). Yield: 6.0 g (61%) of a white solid.

¹H-NMR (400 MHz, Chloroform-d) δ 5.72-4.84 (m, 2H), 2.83 (s, 4H), 2.60 (t, J=7.5 Hz, 2H), 2.16-1.89 (m, 4H), 1.75 (m, J=7.4 Hz, 2H), 1.69-1.51 (m, 2H), 1.51-1.10 (m, 18H), 0.99-0.45 (m, 3H, J=6.8 Hz). MALDI-TOF-MS (CHCA matrix, positive reflector mode): Obs. m/z=(M+H)⁺ 380.28. Calculated: C₂₂H₃₇NO₄ (exact mass 379.27; molecular weight 379.54).

### N⁶-(tert-Butoxycarbonyl)-N²-oleoyl-L-lysine

N-Epsilon-t-butyloxycarbonyl-L-Lysine (551 mg, 2.23 mmol, 1.0 eq) was dissolved in DCM (17 mL) in an inert atmosphere of nitrogen. 2,5-Dioxopyrrolidin-1-yl oleate (851 mg, 2.21 mmol, 0.99 eq) and triethylamine (0.70 mL; 2 eq) were added and stirring was continued for 19 h. DCM (50 mL) was added and the organic phase was washed with aqueous 1M KHSO₄ (3×50 mL), water (3×50 mL), and brine (100 mL). After evaporation to dryness, the crude product was purified by column chromatography on silica using chloroform:methanol:acetic acid (10:10:1) eluent. Yield: 813 mg (71%) of a white solid.

¹H NMR (400 MHz, Chloroform-d) δ 6.60-6.40 (m, 1H), 5.47-5.22 (m, 2H), 4.67 (s, 1H), 4.51 (s, 1H), 3.13 (m, 2H), 2.3-1.5 (m, 12H), 1.44 (s, 9H), 1.5-1.2 (m, 22H), 0.88 (t, J=6.9 Hz, 3H). MALDI-TOF-MS (CHCA matrix, positive reflector mode): Obs. m/z=(M+Na)⁺ 533.39. Calculated: C₂₉H₅₄N₂O₅ (exact mass 510.40; molecular weight 510.74).

### 2,3,5,6-Tetrafluorophenyl N⁶-(tert-butoxycarbonyl)-N²-oleoyl-L-lysinate

N⁶-(tert-Butoxycarbonyl)-N²-oleoyl-L-lysine (623 mg, 1.21 mmol, 1.00 eq) and 2,3,5,6-tetrafluorophenol (302 mg, 1.82 mmol, 1.50 eq) were dissolved in DCM (10 mL) and stirred in a nitrogen atmosphere. The clear solution was cooled with an ice/water bath and EDC·HCl (302 mg, 1.57 mmol, 1.3 eq) was added. Chloroform (75 mL) was added 18h later, and the solution was washed with water (4×50 mL), saturated sodium bicarbonate solution (50 mL), followed by water (50 mL) and brine (50 mL). Purification by column chromatography on silica, elution with 9:1 chloroform:ethyl acetate. Yield: 470 mg (59%) of a white solid.

¹H-NMR (400 MHz, Chloroform-d) δ 7.02 (m, 1H), 6.41-5.98 (m, 1H), 5.34 (m, 2H), 4.93 (m, 1H), 4.61 (m, 1H), 3.15 (m, 2H), 2.26 (t, J=7.6 Hz, 2H), 2.00 (m, 4H), 1.95 (m, 2H), 1.65 (m, 2H), 1.52 (m, 4H), 1.44 (s, 9H), 1.35-1.22 (m, 22H), 0.88 (t, J=6.9 Hz, 3H). ¹⁹F-NMR (Chloroform-d) 5-138.6 (m, 2F), −152.4 (m, 2F).

### N²-(tert-Butoxycarbonyl)-N⁶-oleoyl-L-lysine

(tert-Butoxycarbonyl)-L-lysine (245 mg, 0.99 mmol) was stirred with dry DCM (6 mL) in a nitrogen atmosphere. 2,5-Dioxopyrrolidin-1-yl oleate (380 mg, 1.0 mmol) was added followed by triethylamine (202 mg, 2.0 mmol), and the turbid mixture became clear during stirring overnight. Chloroform (50 mL) was added and the mixture was washed with aqueous 1M KHSO₄ (20 mL), water (20 mL), brine (50 mL), and dried over magnesium sulfate. It was purified by column chromatography on silica, using chloroform:methanol 20:1 as eluent. Yield: 342 mg (67%) of an oil.

¹H NMR (400 MHz, Chloroform-d) δ 5.70 (t, 1H), 5.45-5.17 (m, 2H), 5.25 (m, 1H), 4.30-4.10 (m, two s, 1H), 3.25 (m, 2H), 2.17 (t, J=9.7 Hz, 2H), 1.99 (m, 4H), 1.86 (m, 1H), 1.73 (m, 1H), 1.65-1.15 (m, 31H), 0.87 (t, J=6.9 Hz, 3H).

### 2,3,5,6-Tetrafluorophenyl N²-(tert-butoxycarbonyl)-N⁶-oleoyl-L-lysinate

To a solution of N²-(tert-Butoxycarbonyl)-N⁶-oleoyl-L-lysine (349 mg, 0.67 mmol) and 2,3,5,6-tetrafluorophenol (205 mg, 1.23 mmol, 1.85 eq) in DCM (15 mL) was added EDC·HCl (169 mg, 0.94 mmol, 1.41 eq) in a nitrogen atmosphere. After 21h, DCM (50 mL) was added and the organic phase was washed with aqueous saturated sodium bicarbonate (3×50 mL) followed by brine (50 mL). The solution was evaporated till dryness and the crude product was purified by column chromatography on silica using a chloroform:ethyl acetate gradient from 10:1 to 20:7. Yield: 295 mg (67%) of a white powder.

¹H NMR (400 MHz, CDCl₃) δ 7.02 (m, 1H), 5.49 (t, 1H), 5.40-5.25 (m, 2H), 5.17 (m, 1H), 4.62 and 4.44 (two s, 1H), 3.29 (m, 2H), 2.16 (t, J=9.7 Hz, 2H), 2.00 (m, 4H), 1.89 (m, 2H), 1.65-1.15 (m, 31H), 0.88 (t, J=6.9 Hz, 3H).

### Oleoyl-L-Arginine

THF (32 mL) was added to a vigorously stirred solution of arginine (1.09 g, 6.26 mmol, 1.00 eq) in water (32 mL). Sodium bicarbonate (896 mg, 10.6 mmol, 1.69 eq) was added and after 15 minutes a clear solution was obtained. A solution of 2,5-dioxopyrrolidin-1-yl oleate (2.64 g, 6.95 mmol, 1.11 eq) in THF (32 mL) was added dropwise during 2h. Stirring was continued for 23h, resulting in a clear solution. The THF was removed on a rotary evaporator, and the resulting water phase was transferred to a separatory funnel. A solution of KHSO₄ in water (1.49 g in 260 mL) was added, and shaken with chloroform (590 mL). Water layer pH=3. The chloroform phase was shaken with brine (300 mL). Removal of the chloroform on a rotary evaporator resulted in crude mixture (3.05 g). It was purified by column chromatography on a Grace Reveleris X2 Flash Chromatography System (ELSD detection) with a BQchi 80 g HP column, elution with chloroform:methanol 1:1. The product was dissolved in THF and concentrated (two times: 120 ml, and 80 ml THF) in order to remove residual methanol (necessary before the treatment with HCl). Yield: 2.0 g (TLC, NMR, and MALDI-TOF confirmed the product was pure). Preparation of the HCl salt: the product was dissolved by stirring with DCM (90 ml) and THF (20 ml) in an argon atmosphere, and then cooled on an ice/water bath. A solution of 2M HCl in ether (4.5 mL) was added dropwise in 5 minutes resulting in a clear solution. The solvents were removed on a rotary evaporator and the residue was dried in vacuo. Yield: 2.09 g of a white foam.

¹H NMR (400 MHz, Chloroform-d+TFA) δ 7.13 (d, J=7.5 Hz, 1H), 6.54 (s, 1H), 6.13 (s, 4H), 5.42-5.29 (m, 2H), 4.69 (m, 1H), 3.31 (m, 2H), 2.1-1.55 (m, 12H), 1.48-1.08 (m, 20H), 0.87 (t, J=6.9 Hz, 3H). MALDI-TOF-MS (CHCA matrix, positive reflector mode): Obs. m/z=(M+H)⁺ 439.36. Calculated: C₂₄H₄₆N₄O₃ (exact mass: 438.36; molecular weight of the HCl salt: 475.10).

### A. Functionalization with an Acrylate-Ester (Michael Addition)

### General Example A1. G2-ACR-nC8-16

This general example describes the Michael-addition reaction of G2-PPI—(NH₂)₈ dendrimer with n-octyl acrylate, leading to fully functionalized PPI-dendrimer with 16 octyl-ester groups.

G2 PPI dendrimer (n-butylene core; 0.20 g; 0.258 mmol; 2.06 mmol primary amine groups) was dissolved in iso-propanol (1 mL). An excess of n-octyl acrylate (1.73 mL, 1.53 g, 8.27 mmol; 32 moleqs) was added and the reaction mixture was stirred in a closed vial at 55° C. under an inert atmosphere of nitrogen. The progress of the reaction was monitored with ¹H-NMR. The mixture was stirred for 4 days, after which conversion was complete. The mixture was evaporated in vacuo to dryness, and the residue was stirred in MeCN (15 mL) at 4° C. After a 3-4 hours, the supernatant was decanted (or pipetted off) to remove the excess of acrylate. Washing with MeCN was repeated two more times at 4° C., and one final time at −20° C. (with longer-chain acrylates this can lead to precipitation of the acrylate). The product was dried in vacuo to yield a slightly yellowish oil. Yield: 515 mg (54%). The ¹H-NMR spectrum was in agreement with the desired structure.

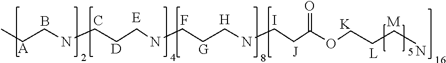

¹H-NMR (400 MHz, Chloroform-d) δ 4.04 (t, J=6.8 Hz, 32H, K), 2.77 (t, J=7.3 Hz, 32H, I), 2.54-2.27 (m, 84H, B, C, E, F, H, J), 1.58 (dt, J=22.2, 6.9 Hz, 60H, A, D, G, L), 1.30 (dd, J=14.0, 7.7 Hz, 160H, M), 1.08-0.64 (m, 48H, N).

MALDI-TOF-MS (CHCA matrix, positive reflector mode): Obs. m/z=(M+H)⁺ 3722.19. Calculated: C₂₁₆H₄₁₆N₁₄O₃₂ (exact mass 3719.14; molecular weight 3721.77).

### Example A2. G1-ACR-nC8-8

The reaction between G1-PPI—(NH₂)₄ (n-butylene core) and n-octyl acrylate was performed in a similar way as done for Example A1. Yield: 758 mg (67%). The ¹H-NMR spectrum was in agreement with the desired structure.

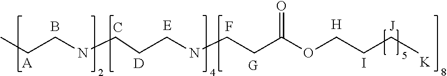

¹H NMR (400 MHz, Chloroform-d) δ 4.05 (t, J=6.8 Hz, 16H, H), 2.77 (t, J=7.4 Hz, 16H, F), 2.40 (dt, J=23.2, 7.5 Hz, 36H, B, C, E, G), 1.58 (dt, J=23.6, 6.7 Hz, 28H, A, D, 1), 1.47-1.07 (m, 80H, J), 1.07-0.57 (m, 24H, K).

MALDI-TOF-MS (CHCA matrix, positive reflector mode): Obs. m/z=(M+H)⁺ 1791.52, (M+Na)⁺ 1813.52, (M+K)⁺ 1829.49. Calculated: C₁₀₄H₂₀₀N₆O₁₆ (exact mass 1789.50; molecular weight 1790.77).

### Example A3. G1-ACR-nC14-8

The reaction between G1-PPI-(NH₂)₄(n-butylene core) and n-tetradecyl acrylate was performed in a similar way as done for Example A1. However, HFIP was used as solvent. Work-up: after evaporation in vacuo of the HFIP at 50° C. the reaction mixture was co-evaporated with toluene three times at 50° C. The residue was purified by treatment with MeCN in a similar way as described in Example 1. Yield: 615 mg (75%). ¹H-NMR was in agreement with the desired structure.

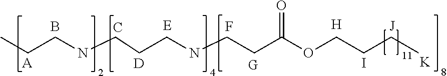

¹H NMR (399 MHz, Chloroform-d) δ 4.04 (t, J=6.9 Hz, 16H, H), 2.91-2.70 (m, 16H, F), 2.43 (t, J=7.3 Hz, 36H, B, C, E, G), 1.61 (t, J=7.0 Hz, 28H, A, D, 1), 1.26 (m, 176H, J), 0.99-0.74 (m, 24H, K).

MALDI-TOF-MS (CHCA matrix, positive reflector mode): Obs. m/z (M+H)⁺ 2464.31, (M+Na)⁺ 2489.30. Calculated: C₁₅₂H₂₉₆N₆O₁₆ (exact mass 2462.25; molecular weight 2464.07).

### Example A4. G1-ACR-nC4-8

The reaction between G1-PPI—(NH₂)₄(n-butylene core) and n-butyl acrylate was performed in the a similar way as done for Example A1. Work-up: the acrylate excess was removed by evaporation under vacuum. The acrylate could also be removed by stirring the reaction mixture with amine-functionalized silica scavenger (excess butyl acrylate gets immobilized by reaction with the silica-bound amine groups). Yield: 450 mg (53%). ¹H-NMR was in agreement with the desired structure.

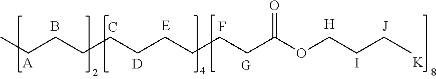

¹H NMR (399 MHz, Chloroform-d) δ 4.06 (t, J=6.7 Hz, 16H, H), 2.77 (t, J=7.3 Hz, 16H, F), 2.55-2.14 (m, 36H, B, C, E, G), 1.87-1.46 (m, 28H, A, D, 1), 1.46-1.29 (m, 16H, J), 0.93 (t, J=7.4 Hz, 24H, K).

MALDI-TOF-MS (CHCA matrix, positive reflector mode): Obs. m/z=(M+H)⁺ 1342.02, (M+Na)⁺ 1364.02. Calculated: C₇₂H₁₃₆N₆O₁₆ (exact mass 1341.00; molecular weight 1341.91).

### Example A5. G2-ACR-nC14-16

The reaction between G2-PPI—(NH₂)₈ (n-butylene core) and n-tetradecyl acrylate was performed in a similar way as done for Example A3. Yield: 580 mg (60%). ¹H-NMR was in agreement with the desired structure.

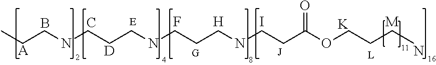

¹H NMR (399 MHz, Chloroform-d) δ 4.04 (s, 32H, K), 2.76 (t, J=7.4 Hz, 32H, I), 2.41 (h, J=8.0, 7.3 Hz, 84H, B, C, E, F, H, J), 1.60 (q, J=7.0 Hz, 60H, A, D, G, L), 1.26 (s, 352H, M), 0.88 (s, 48H, N).

MALDI-TOF-MS (CHCA matrix, positive reflector mode): Obs. m/z=(M+H)⁺ 5068.95. Calculated: C₃₁₂H₆₀₈N₁₄O₃₂ (exact mass 5064.64; molecular weight 5068.36).

### Example A6. G1-ACR-isoC10-8

The reaction between G1-PPI (DAB-Am-4) and iso-decyl acrylate was performed in a similar way as done for Example A1. Yield: 0.85 g (67%). The ¹H-NMR spectrum was in agreement with the desired structure.

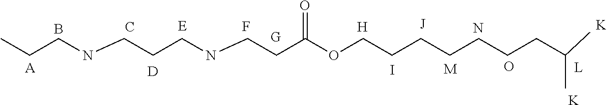

¹H NMR (399 MHz, Chloroform-d) δ 4.23-3.74 (m, 16H, H), 2.77 (t, J=7.3 Hz, 16H, F), 2.63 (s, 1H), 2.53-2.28 (m, 35H, G, C, E, B), 2.06-1.73 (m, 2H), 1.73-0.97 (m, 90H, A, D, I, J, M, O, L), 0.97-0.54 (m, 75H, K, N, P). MALDI-TOF-MS (CHCA matrix, positive reflector mode): Obs. m/z=(M+H)⁺ 2015.17. Small peak with m/z=M+H⁺, 2029.76 (extra CH₂). Calculated: C₁₂₀H₂₃₂N₆O₁₆ (exact mass 2013.75; molecular weight 2015.20).

### Example A7. G1(PPeI)-ACR-C8-8

N,N,N′,N′-Tetra(5-aminopentyl)-1,4-diaminobutane (G1-PPeI, see above; 200 mg, 0.47 mmol, 1 eq) and n-octyl acrylate (1.6 mL, 1.4 g, 7.6 mmol, 16 eq) were dissolved in propan-2-ol (IPA; 1 mL). The mixture was heated to 60° C. for 112h and the reaction was monitored using ¹H-NMR. The mixture was cooled down and concentrated in vacuo. The viscous oil was stirred in MeCN, cooled down to −20° C. and the oil was allowed to settle at the bottom of the flask. The supernatant was decanted, and the procedure was repeated three times to give oily G1(PPeI)-ACR-C8-8 product.

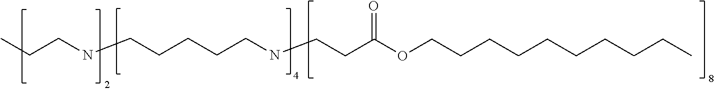

¹H NMR (400 MHz, Chloroform-d) δ 4.05 (t, J=6.8 Hz, 16H), 2.76 (t, J=7.3 Hz, 16H), 2.41 (q, J=8.6, 8.1 Hz, 36H), 1.61 (p, J=6.9 Hz, 16H), 1.43 (p, J=7.5 Hz, 20H), 1.37-1.15 (m, 88H), 0.92-0.84 (t, J=6.8 Hz, 24H). MALDI-TOF-MS (CHCA matrix, positive reflector mode): Obs. m/z=(M+H)⁺ 1903.63, (M+Na)⁺ 1925.60 Calculated: C₁₁₂H₂₁₆N₆O₁₆ (exact mass 1901.63; molecular weight 1902.99).

### Example A8. G2(PAMAM-C2)-ACR-C8-16

To a reaction tube with a stirred solution of G2-PAMAM-C2 (ethylene-diamine core; 8 amine end groups; 200 mg, 0.14 mmol) in propan-2-ol (IPA; 1 mL) was added n-octyl-acrylate (825 mg, 4.48 mmol, 32 eq). The reaction mixture was heated to 60° C. for 304 h. The reaction mixture was concentrated in vacuo. The residue was stirred in MeCN and cooled down to −20° C., allowing the product to settle on the bottom of the flask. The supernatant was decanted and the procedure was repeated four times yielding a yellow viscous oil (301 mg, 68.8 μmol, 49%).

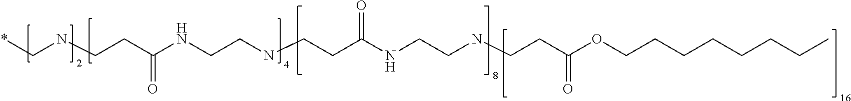

¹H NMR (400 MHz, Chloroform-d) δ 7.72 (s, 12H), 4.04 (t, J=6.8 Hz, 32H), 3.27 (q, J=5.8 Hz, 24H), 2.79 (dt, J=13.8, 6.8 Hz, 56H), 2.57 (dt, J=12.1, 5.9 Hz, 28H), 2.42 (t, J=6.9 Hz, 32H), 2.35 (d, J=6.7 Hz, 24H), 1.62 (p, J=6.8 Hz, 32H), 1.41-1.18 (m, 160H), 0.95-0.82 (m, 48H). MALDI-TOF-MS (DCBT matrix, positive reflector mode): Obs. m/z=(M+H)⁺ 4378.32 Calculated: C₂₃₈H₄₄₈N₂₆O₄₄ (exact mass 4375.36; molecular weight 4378.34).

### Example A9. Bis(3-Aminopropyl)Amine-ACR-C8-5

n-Octyl-acrylate (1.84 g, 10 mmol, 10 eq) was added to a stirred solution of bis(3-aminopropyl)amine (131 mg, 1 mmol) in iso-propanol (1 mL). The reaction mixture was heated to 60° C. for 136h and was thereafter concentrated in vacuo. The residue was stirred in MeCN and was cooled down to −20° C. allowing the product to phase separate from the MeCN layer. The supernatant was carefully removed using a pipette and the procedure was repeated five times. The residue was dried in vacuo yielding a clear colorless oil (769 mg, 0.73 mmol, 73%).

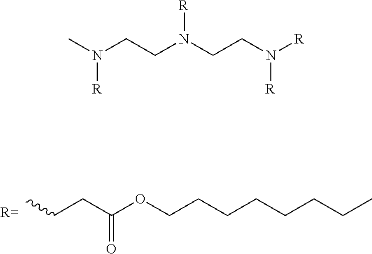

¹H NMR (400 MHz, Chloroform-d) δ 4.05 (t, J=6.8 Hz, 10H), 2.76 (td, J=7.4, 3.8 Hz, 10H), 2.41 (dt, J=10.7, 6.8 Hz, 18H), 1.74-1.48 (m, 14H), 1.42-1.16 (m, 50H), 0.95-0.80 (m, 15H). ¹³C NMR (101 MHz, CDCl₃) δ 172.9, 172.7, 64.6, 64.6, 51.8, 51.8, 49.3, 49.2, 32.6, 32.3, 31.8, 29.3, 29.2, 28.7, 28.7, 25.9, 24.8, 22.7, 14.1. MALDI-TOF-MS (CHCA matrix, positive reflector mode): Obs. m/z=(M+H)⁺ 1052.85, (M+Na)⁺ 1074.84, (M+K)⁺ 1090.80. Calculated: C₆₁H₁₁₇N₃O₁₀ (exact mass 1051.87; molecular weight 1052.62).

### Example A10. Bis(2-aminoethyl)amine-ACR-C8-5

n-Octyl-acrylate (1.84 g, 10 mmol, 10 eq) and bis(2-aminoethyl)amine (103 mg, 1 mmol) were reacted in iso-propanol in a similar way as described for Example A9. Yield: 674 mg (0.66 mmol, 66%).

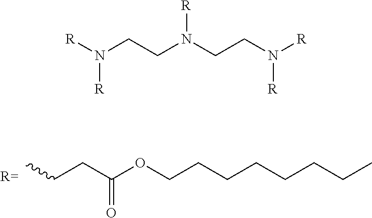

¹H NMR (399 MHz, Chloroform-d) δ 4.05 (t, J=6.8 Hz, 10H), 2.78 (t, J=7.2 Hz, 10H), 2.51 (s, 8H), 2.43 (t, J=7.3 Hz, 10H), 1.62 (p, J=7.0 Hz, 10H), 1.39-1.19 (m, 50H), 1.00-0.76 (m, 15H). ¹³C NMR (100 MHz, CDCl₃) δ 172.7, 172.6, 64.6, 52.9, 52.3, 50.5, 49.8, 32.7, 32.7, 31.8, 29.3, 29.2, 28.7, 28.7, 25.9, 22.7, 14.1. MALDI-TOF-MS (CHCA matrix, positive reflector mode): Obs. m/z=(M+H)⁺ 1024.82, (M+Na)⁺ 1046.80, (M+K)⁺ 1082.78. Calculated: C₅₉H₁₁₃N₃O₁₀ (exact mass 1023.84; molecular weight 1024.56).

### Example A11. Bis-(3-aminopropyl)methylamine-ACR-C8-4

n-Octyl-acrylate (1.47 g, 8 mmol, 8 eq) and bis-N,N-(3-aminopropyl)methylamine (145 mg, 1 mmol) were reacted in iso-propanol in a similar way as described for Example A9. Yield: 552 mg (0.63 mmol, 63%).

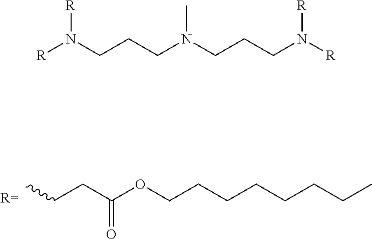

¹H NMR (400 MHz, Chloroform-d) δ 4.05 (t, J=6.8 Hz, 8H), 2.77 (t, J=7.3 Hz, 8H), 2.43 (td, J=7.2, 2.8 Hz, 12H), 2.29 (t, J=7.3 Hz, 4H), 2.18 (s, 3H), 1.61 (q, 6.7 Hz, 12H), 1.30 (m, 40H), 0.99-0.77 (m, 12H). ¹³C NMR (101 MHz, CDCl₃) δ 172.7, 64.6, 55.8, 51.9, 49.3, 42.1, 32.7, 31.8, 29.3, 29.2, 28.7, 25.9, 25.1, 22.7, 14.1. MALDI-TOF-MS (CHCA matrix, positive reflector mode): Obs. m/z=(M+H)⁺ 882.71, (M+Na)⁺ 904.72. Calculated: C₅₁H₉₉N₃O₈ (exact mass 881.74; molecular weight 882.37).

### Example A12. Bis(3-aminopropyl)amine-ACR-(2-ethyl-hexyl)-5

2-Ethyl-hexyl-acrylate (1.4 g, 7.5 mmol, 10 eq) was added to a stirred solution of bis(3-aminopropyl)amine (100 mg, 0.75 mmol) in iso-propanol (2 mL). Components were reacted in a similar way as described for Example A9. Yield after work-up: 278 mg (0.26 mmol, 35%).

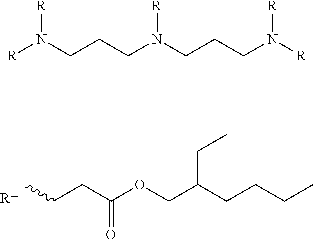

¹H NMR (399 MHz, Chloroform-d) δ 4.19-3.78 (m, 10H), 2.77 (t, J=7.3 Hz, 10H), 2.56-2.25 (m, 17H), 2.01 (s, 3H), 1.67 (s, 1H), 1.64-1.48 (m, 9H), 1.48-1.11 (m, 40H), 0.89 (td, J=7.1, 2.8 Hz, 30H). MALDI-TOF-MS (CHCA matrix, positive reflector mode): Obs. m/z=(M+H)⁺ 1052.88, (M+Na)⁺ 1074.86. Calculated: C₅₉H₁₁₃N₃O₁₀ (exact mass 1051.87; molecular weight 1052.62).

### Example A13. G1-ACR-(2-ethyl-hexyl)-8

The reaction between G1-PPI (DAB-Am-4) and 2-ethyl-hexyl acrylate was performed in a similar way as done for Example A1. Yield: 0.71 g (0.4 mmol, 73%). The ¹H-NMR spectrum was in agreement with the desired structure.

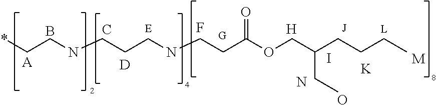

¹H NMR (399 MHz, Chloroform-d) δ 4.36-3.57 (m, 16H, H), 2.78 (t, J=7.4 Hz, 16H, F), 2.41 (dt, J=25.0, 7.5 Hz, 35H, G, C, E, B), 1.69-1.48 (m, 16H), 1.48-1.11 (m, 68H), 0.89 (td, J=7.1, 2.8 Hz, 48H, M, O). MALDI-TOF-MS (CHCA matrix, positive reflector mode): Obs. m/z=(M+H)⁺ 1791.55, (M+Na)⁺ 1813.53. Calculated: C₁₂₀H₂₃₂N₆O₁₆ (exact mass 1789.50; molecular weight 1790.77).

### Example A14. G1(PBuI)-ACR-C8-8

N,N,N′,N′-Tetra(4-aminobutyl)-1,4-diaminobutane (G1-PBuI) and n-octyl acrylate were dissolved in propan-2-ol (IPA; 1 mL). The reaction and work-up was done similarly as described for Example A7 to provide oily G1(PBuI)-ACR-C8-8 product.

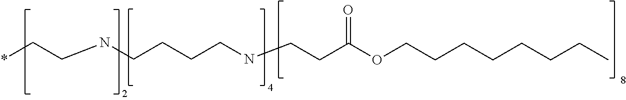

¹H NMR (400 MHz, Chloroform-d) δ 4.05 (t, 16H), 2.76 (t, 16H), 2.41 (q, 36H), 1.61 (p, 16H), 1.43 (p, 20H), 1.37-1.15 (m, 80H), 0.92-0.84 (t, 24H). MALDI-TOF-MS (CHCA matrix, positive reflector mode): Obs. m/z=(M+H)⁺ 1847, (M+Na)⁺ 1869. Calculated: C₁₀₈H₂₀₈N₆O₁₆ (exact mass 1846; molecular weight 1846).

### Example A15. G1(PAMAM-C2)-ACR-(2-ethyl-hexyl)-8

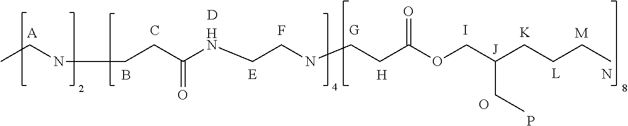

To a reaction tube with a stirred solution of G1-PAMAM-C2 (ethylene-diamine core; 4 amine end groups; 165 mg, 0.32 mmol) in propan-2-ol (IPA; 2 mL) was added 2-ethyl-hexylacrylate (589 mg, 3.2 mmol, 10 eq). More 2-ethyl-hexylacrylate was later added (600 mg, 3.3 mmol, 10 eq). The reaction mixture was heated to 60° C. for 408 h. The reaction mixture was concentrated in vacuo. The residue was evaporated further with oil pump to remove excess of 2-ethyl-hexylacrylate (at 90° C. and 0 mbar). The crude product still contained a small amount of acrylate that was further removed using a short silica column (starting 2% MeOH/CHCl₃ to 10% MeOH/CHCl₃) yielding a yellow viscous oil (270 mg, 0.13 mol, 41%).

¹H NMR (400 MHz, Chloroform-d) δ 7.20 (t, J=5.5 Hz, 3H), 4.19-3.78 (m, 16H, I), 3.27 (q, J=6.0 Hz, 8H, B), 2.78 (dt, J=15.0, 6.9 Hz, 22H, F, G), 2.57 (t, J=6.4 Hz, 9H, C), 2.52 (s, 3H, A), 2.44 (t, J=7.1 Hz, 15H, H), 2.34 (t, J=6.4 Hz, 7H, E), 1.63-1.47 (m, 8H, J), 1.44-1.12 (m, 64H, K, L, M, O), 0.89 (td, J=7.5, 7.0, 3.0 Hz, 48H, N, P). MALDI-TOF-MS (CHCA matrix, positive reflector mode): Obs. m/z=(M+H)⁺ 1991.56, (M+Na)⁺ 2013.53. Calculated: C₁₁₀H₂₀₃N₁₀O₂₀ (exact mass 1989.56; molecular weight 1990.92).

### B. Selective Functionalization of Primary Amines with an Acrylate-Ester (Michael Addition) Followed by Acylation of the Remaining Secondary Amines

The basis for the below materials was found by the observation that reactions of acrylates with primary amine dendrimers proceeded much faster than those with the secondary amine dendrimers that were formed. Accordingly, this selectivity could be used to cap the formed secondary amine intermediates by acylation.

### General Example B1. G1-ACR-Citro-4/Ac-4

This general example describes the Michael-addition reaction of G1-PPI—(NH₂)₄ dendrimer with (S)-3,7-dimethyloct-6-en-1-yl acrylate, followed by reaction with acetic anhydride, leading to G1 PPI-dendrimer with 4 (S)-citronellyl ester groups and 4 acyl-amide groups.

G1 PPI dendrimer (n-butylene core; 0.20 g; 0.631 mmol) was dissolved in iso-propanol (1 mL) and (S)-3,7-dimethyloct-6-en-1-yl acrylate (0.513 g; 2.44 mmol) was added. The mixture was stirred overnight in a closed vial at 55° C. under an inert atmosphere of nitrogen which resulted in full conversion of the acrylate. According to ¹H-NMR the acrylate reacted much faster with primary amines, thus resulting in (almost) exclusive mono-functionalization of each of the primary amines. The mixture was evaporated to dryness and the residue was dissolved in dichloromethane (DCM; 1 mL). Subsequently, triethylamine (0.36 mL, 0.268 g; 2.65 mmol) was added, followed by acetic anhydride (0.271 g; 2.65 mmol). The mixture was stirred in a closed vial under an atmosphere of nitrogen at 20° C. for 4 hours, was then diluted with DCM and subsequently washed with NaHCO₃ (aq.) and brine. The organic layer was dried over Na₂SO₄, filtered, and the filtrate was evaporated to dryness. Co-evaporation with DCM was done to remove traces of acetic anhydride and acetic acid. Yield: 623 mg (74%). The ¹H-NMR spectrum was in agreement with the desired structure.

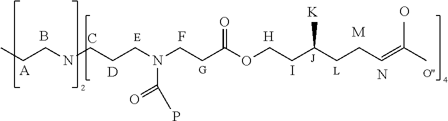

¹H NMR (399 MHz, Chloroform-d) δ 5.08 (t, J=7.0, 1.4 Hz, 4H, N), 4.31-3.87 (m, 8H, H), 3.58 (dt, J=10.9, 7.2 Hz, 8H, F), 3.31 (q, J=8.9, 8.3 Hz, 8H, E), 2.70-2.50 (m, 8H, C), 2.42 (d, J=7.3 Hz, 12H, B, G), 2.18-2.07 (m, 12H, P), 2.07-1.92 (m, 8H, M), 1.89-1.63 (m, 24H, O, O″), 1.63-1.08 (m, 32H, A, D, I, J, L), 0.91 (dd, J=6.6, 4.4 Hz, 12H, K).

MALDI-TOF-MS (CHCA matrix, positive reflector mode): Obs. m/z=(M+H)⁺ 1326.02, (M+Na)⁺ 1348.00. Calculated: C₇₆H₁₃₆N₆O₁₂ (exact mass 1325.01; molecular weight 1325.95).

### Example B2. G1-ACR-nC14-4/Ac-4

The reaction between G1-PPI—(NH₂)₄ (n-butylene core) and n-tetradecyl acrylate and acetic anhydride was performed in a similar way as done for Example E1. Yield: 689 mg (70%). The ¹H-NMR spectrum was in agreement with the desired structure. MALDI-TOF-MS (CHCA matrix, positive reflector mode): Obs. m/z=(M+H)⁺ 1558.34, (M+Na)⁺ 1580.33 (dominant peak). Calculated: C₉₂H₁₇₆N₆O₁₂ (exact mass 1557.33; molecular weight 1558.45). Also observed: small peak with m/z=1786.33 (5 tetradecyl tails and 3 acetyl groups).

### Example B3. G2-ACR-Citro-8/Ac-8

The reaction between G2-PPI—(NH₂)₈ (n-butylene core) and (S)-3,7-dimethyloct-6-en-1-yl acrylate and acetic anhydride was performed in a similar way as done for Example 1. Yield: 520 mg (72%). The ¹H-NMR spectrum was in agreement with the desired structure. MALDI-TOF-MS (CHCA matrix, positive reflector mode): Obs. m/z=(M+H)⁺ 2792.18, (M+Na)⁺ 2814.16. Calculated: C₁₆₀H₂₈₈N₁₄O₂₄ (exact mass 2790.17; molecular weight 2792.14).

### Example B4. G2-ACR-nC14-8/Ac-8

The reaction between G2-PPI—(NH₂)₈ (n-butylene core) and n-tetradecyl acrylate and acetic anhydride was performed in a similar way as done for Example E1. Yield: 631 mg (75%). The ¹H-NMR spectrum was in agreement with the desired structure.

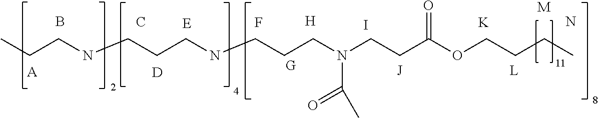

¹H NMR (399 MHz, Chloroform-d) δ 4.06 (dt, J=13.5, 6.8 Hz, 16H, K), 3.58 (dt, J=13.9, 6.7 Hz, 16H, I), 3.31 (q, J=9.5, 9.0 Hz, 16H, H), 2.60 (ddd, J=10.4, 6.1, 2.4 Hz, 16H, J), 2.40 (p, J=9.9, 8.5 Hz, 36H, B, C, E, F), 2.10 (d, J=10.7 Hz, 24H, O), 1.86-1.46 (m, 44H, A, D, G, L), 1.46-1.07 (m, 176H, M), 1.07-0.58 (m, 24H, N).

MALDI-TOF-MS (CHCA matrix, positive reflector mode): Obs. m/z=(M+H)⁺ 3257.80, (M+Na)⁺ 3279.79. Calculated: C₁₉₂H₃₆₈N₁₄O₂₄ (exact mass 3254.80; molecular weight 3257.13). Also observed: small peak with m/z=3484.01 (9 tetradecyl tails and 7 acetyl groups).

### C. Functionalization of Primary Amines with a First Acrylate-Ester and then with Another Second Acrylate-Ester (Michael Additions)

### General Example C1. G3-ACR-nC14-16/C1-16

This general example describes the Michael-addition reaction of G3-PPI—(NH₂)₁₆ dendrimer with n-tetradecyl acrylate, followed by reaction with methyl acrylate, leading to PPI-dendrimer with 16 n-tetradecyl-ester groups and 16 methyl ester groups.

G3 PPI dendrimer (0.20 g, 0.119 mmol) was dissolved in isopropanol (1 mL) and tetradecyl acrylate (0.513 g, 1.91 mmol, 16 moleqs) was added. The mixture was stirred in a closed vial at 55° C. under an inert atmosphere of nitrogen for 5 hours which resulted in full conversion of the primary amines (a slight amount of acrylate was still present). According to the ¹H-NMR, the acrylate reacts much faster with primary amines, thus resulting in almost exclusive mono-functionalization of each of the primary amine groups. Subsequently, methyl acrylate was added (0.27 mL, 0.244 g, 2.83 mmol, 24 moleqs), and the mixture was stirred at 55° C. The progress of the reaction was monitored with ¹H-NMR. After 1 day, an extra amount of methyl acrylate (0.14 mL, 0.122 g, 1.42 mmol, 12 eq.) was added. After stirring at 55° C. for a total of 4 days the reaction was complete, and the mixture was evaporated to dryness. The crude product was stirred in MeCN and was put at 0° C. for a few hours to induce full phase separation. Subsequently, the supernatant was decanted. This procedure was repeated three times to remove any unreacted acrylate monomer. Yield: 575 mg (66%). The ¹H-NMR spectrum was in agreement with the desired structure.

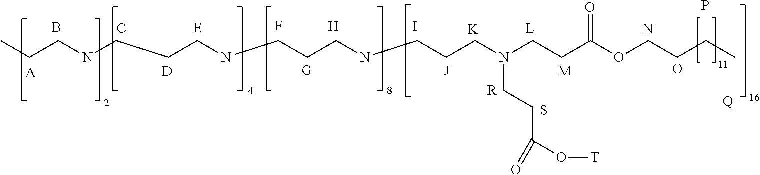

¹H NMR (400 MHz, Chloroform-d) δ 4.04 (t, J=6.9 Hz, 32H, N), 3.65 (s, 48H, T), 2.76 (t, J=7.3 Hz, 64H, L, R), 2.63-2.18 (m, 180H, B, C, E, F, H, I, K, M, S), 1.80-1.44 (m, 92H, A, D, G, J, O), 1.26 (s, 352H, P), 0.88 (t, J=6.7 Hz, 48H, Q). MALDI-TOF-MS: too high MW for measurement. Calculated: C₄₂₄H₃₁₆N₃₀O₆₄ (exact mass 7354.15; molecular weight 7359.34).

### Example C2. G1-ACR-nC14-4/C1-4

The reaction between G1-PPI—(NH₂)₄ (n-butylene core) and n-tetradecyl acrylate and methyl acrylate was performed in a similar way as Example C1. Yield: 464 mg (84%). The ¹H-NMR spectrum was in agreement with the desired structure.

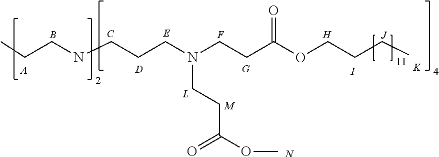

¹H NMR (399 MHz, Chloroform-d) δ 4.05 (t, J=6.8 Hz, 8H, H), 3.66 (s, 12H, N), 2.77 (t, J=7.3 Hz, 16H, F, L), 2.41 (ddt, J=22.6, 14.6, 7.0 Hz, 32H, C, E, G, M), 1.90-1.50 (m, 20H, A, D. I), 1.50-1.20 (m, 96H, I, J), 1.03-0.72 (m, 12H, K).

MALDI-TOF-MS (CHCA matrix, positive reflector mode): Obs. m/z=(M+H)⁺ 1735.44. Small peak with m/z=M+H)⁺ 1917.63 (5 C14-chains and 3 C1 chains). Calculated: C₁₀₀H₁₉₂N₆O₁₆ (exact mass 1733.44; molecular weight 1734.66).

### D. The Functionalization of Amine-Terminal PPI-Dendrimers with an Acrylate-Ester (Michael Addition), with Quaternization of Interior Amines

### Example D1. G1-Boc-4

G1 PPI dendrimer (0.513 g, 1.62 mmol) was dissolved in DCM (20 mL) and the solution was cooled in an ice-salt bath of −10° C. Boc-anhydride (1.48 g, 6.79 mmol) was added. A precipitate formed almost immediately. The mixture was stirred for 4 hours at room temperature after which the reaction was complete. The mixture was filtered and the filtrate was stirred with amine-functionalized silica scavenger overnight to remove excess Boc-anhydride. After filtration, the filtrate was evaporated to dryness to yield the product. Yield: 1.16 gram (81%). The ¹H-NMR spectrum was in agreement with the desired structure.

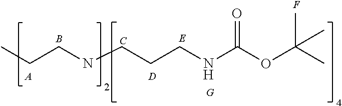

¹H NMR (400 MHz, Chloroform-d) δ 5.59-4.89 (m, 4H, G), 3.13 (q, J=6.4 Hz, 8H, E), 2.40 (dt, J=22.0, 6.6 Hz, 12H, B, C), 1.61 (p, J=6.7 Hz, 8H, D), 1.40 (s, 40H, A, F).

### Example D2. G1-Boc-4 (2 MeCl)

Boc-protected G1 PPI dendrimer G1-Boc-4 (0.75 g, 1.04 mmol) was dissolved in DCM (3 mL) and methyl iodide (1.78 g, 0.78 mL, 12.55 mmol) was added. The mixture was stirred at 45° C. overnight, and then evaporated to dryness yielding a foam which was dissolved in CHCl₃ and precipitated into pentane. The supernatant was decanted and the residue was stirred with pentane again and decanted. The product contained iodide as the counter anion, and these were exchanged for chlorides using an ion exchange column (Lewatit monoplus M600 resin 1.3 meq/mL), applying methanol as eluent. The product was checked for the presence of iodide by dissolving about 5 mg in demi-water (1 mL) and adding a few drops of 35% H₂O₂(aq). No yellow color, indicating iodine I₂ formation, was observed; to this mixture a fresh starch solution (1 mL) was added, and no blue discoloring was observed either. Yield: 715 mg (84%). The ¹H-NMR spectrum was in agreement with the desired structure.

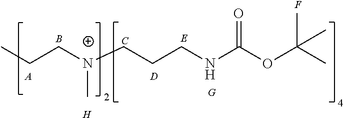

¹H NMR (399 MHz, Chloroform-d) δ 5.51 (s, 4H, G), 3.67 (s, 4H, B), 3.39 (s, 8H, C), 3.27 (s, 8H, E), 3.16 (s, 6H, H), 2.10 (d, J=14.2 Hz, 12H, A, D), 1.44 (s, 36H, F).

### Example D3. G1-NH₂-4 (2 MeCl)

G1-Boc-4 (2 MeCl), about 800 mg, was mixed with 4M HCl in dioxane (about 5 mL), and this gave a precipitate. In order to increase solubility, 3M HCl in methanol was added (about 5 mL). The reaction mixture was stirred overnight at room temperature. ¹H-NMR indicated complete deprotection. The mixture was evaporated to dryness, the residue was co-evaporated with iso-propanol to yield a hygroscopic solid (the compound has a poor solubility in iso-propanol). Yield: 500 mg (122%), due to included HCl and/or water. The ¹H-NMR spectrum was in agreement with the desired structure.

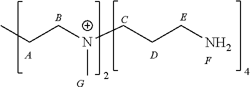

¹H NMR (400 MHz, Methanol-d4) δ 3.86-3.63 (b, F), 3.63-3.44 (m, 12H, B, C), 3.19 (s, 6H, G), 3.10 (t, J=7.3 Hz, 8H, E), 2.26 (d, J=10.7 Hz, 8H, D), 1.97 (s, 4H, A).

### Example D4: G1-ACR-nC14-8 (2 MeCl)

G1-NH₂ (2 MeCl) from Example D3 (300 mg, 1.77*10⁻⁵ mol) was dissolved in HFIP (2 mL) and n-tetradecyl acrylate (2.04 mL, 2 eq. per primary amine) was added. The reaction mixture was a clear solution. The mixture was stirred at 55° C. for one hour, but no reaction took place. Triethylamine (1.6 mL) was added as base and the mixture was stirred for 24 hours. The reaction was complete according to ¹H-NMR. The reaction mixture was evaporated to dryness. The residue was stirred in MeCN, put at 0° C., and the supernatant was decanted. This procedure was performed three times. The residue was then stirred in 1/1 EtOAc/hexane. The precipitate was filtered off, and this still contained 1% of tetradecyl acrylate which was removed by stirring in EtOAc and filtration. The residue was then put on a ion exchange resin to exchange residual iodide for chloride (1/1 CHCl₃/MeOH as eluent). Yield: 468 mg (102%). The ¹H-NMR spectrum was in agreement with the desired structure.

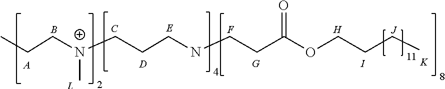

¹H NMR (399 MHz, Chloroform-d) δ 4.02 (t, J=6.8 Hz, 16H, H), 3.84 (d, J=7.5 Hz, 4H, B), 3.38 (dd, J=10.4, 6.2 Hz, 8H, C), 3.19 (s, 6H, L), 2.71 (td, J=6.7, 2.5 Hz, 16H, F), 2.57 (t, J=6.0 Hz, 8H, E), 2.40 (t, J=6.6 Hz, 16H, G), 2.19 (d, J=7.7 Hz, 4H, A), 2.02 (dt, J=13.2, 6.4 Hz, 8H, D), 1.62 (q, J=6.9 Hz, 16H, 1), 1.52-1.02 (m, 176H, J), 0.88 (t, J=6.7 Hz, 24H, K).

### Example D5. G2-Boc-8

The reaction between G2 PPI-dendrimer (DAB-Am-8) and Boc-anhydride was performed in a similar way as shown in Example D1. Yield: 796 mg (99%). The ¹H-NMR spectrum was in agreement with the desired structure.

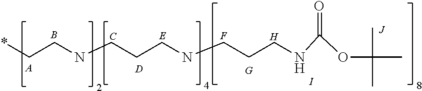

¹H NMR (399 MHz, Methanol-d₄) δ 7.90 (s, 4H, 1), 3.07 (t, J=6.8 Hz, 16H, H), 2.46 (p, J=8.5, 7.6 Hz, 35H, B, E, F), 1.63 (p, J=6.8 Hz, 24H, D, G), 1.43 (s, 77H, A, J).

### Example D6. G2-Boc-8 (6 MeCl)

The reaction between G2-Boc-8 and methyl iodide with subsequent anion exchange was performed in a similar way as shown in Example D2. Yield: 699 mg (73%). The ¹H-NMR spectrum was in agreement with the desired structure.

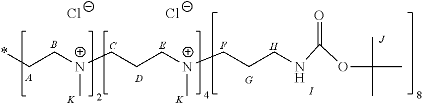

¹H NMR (399 MHz, Methanol-d₄) δ 3.71 (d, J=8.4 Hz, 4H, 1), 3.66-3.38 (m, 34H, B, C, E, F), 3.29-3.04 (m, 43H, K, H, G), 2.43 (q, J=8.2 Hz, 8H, D), 1.99 (h, J=8.5, 6.9 Hz, 21H, A, G), 1.44 (s, 79H, J).

### Example D7. G2-NH₂-8 (6 MeCl)

The reaction between G2-Boc-8 (6 MeCl) and HCl was performed in a similar way as shown in Example D3. Yield: 478 mg (93%). The ¹H-NMR spectrum was in agreement with the desired structure. This product can be used to react with acryl-esters, or acryl-amides or epoxides, etc.

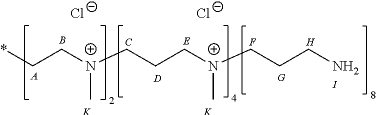

¹H NMR (399 MHz, Deuterium Oxide) δ 3.58 (t, J=8.9 Hz, 36H, B, C, E, F), 3.25 (d, J=4.4 Hz, 18H) 1, J, 3.15 (t, J=7.7 Hz, 16H, H), 2.42 (s, 8H, D), 2.23 (dd, J=14.2, 5.5 Hz, 16H, G), 1.98 (s, 4H, A).

### E. The Functionalization of Amines with Acryl-Amides (Michael-Addition)

### General Example E1. G1-ACRAmAlc-8

This general example describes the Michael-addition reaction of G1-PPI—(NH₂)₄ dendrimer with N-(2-hydroxyethyl)-N-methyl acrylamide, leading to fully functionalized PPI-dendrimer with 8 N-(2-hydroxyethyl)-N-methyl amide groups.

N-(2-hydroxyethyl)-N-methyl acrylamide was prepared as according to Moszner, Macromol. Chem. Phys 2007, 208, 529-540 DOI:10.1002/MACP.200600513. G1 PPI dendrimer (0.10 g, 0.315 mmol) was dissolved in iso-propanol (1 mL) and an excess of N-(2-hydroxyethyl)-N-methylacrylamide (0.515 g, 3.98 mmol, 3 moleqs per primary amine) was added. The reaction mixture was stirred at a temperature of 55° C. for 2 days in a closed vial and under a nitrogen atmosphere, with monitoring of the conversion by ¹H-NMR. The mixture was evaporated to dryness, the residue was stirred in MeCN, and the mixture was then put at 4° C. for several hours. The supernatant was decanted to remove excess of acrylamide reactant. This washing step with MeCN was repeated twice at 4° C. and one time at −20° C. The product was dried in vacuo affording a slightly yellowish oil. Yield: 367 mg (86%). The ¹H-NMR spectrum was in agreement with the desired structure.

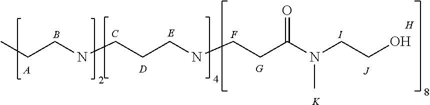

¹H NMR (399 MHz, Chloroform-d) δ 4.89 (s, 8H, H), 3.71 (t, J=5.4 Hz, 16H, J), 3.50 (dt, J=10.3, 5.1 Hz, 16H, 1), 3.12 (s, 12H, K), 2.93 (s, 12H, K), 2.79 (d, J=7.0 Hz, 16H, F), 2.63-2.54 (m, 8H, E), 2.49 (d, J=7.5 Hz, 24H, B, C, G), 1.79 (s, 8H, D), 1.46 (s, 4H, A).

MALDI-TOF-MS (CHCA matrix, positive reflector mode): Obs. m/z=(M+H)⁺ 1349.98, (M+Na)⁺ 1371.96. Calculated: C₆₄H₁₂₈N₁₄O₁₆ (exact mass 1348.96; molecular weight 1349.81).

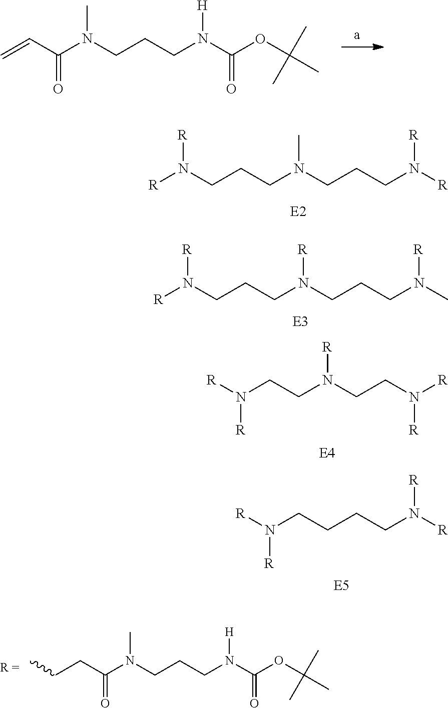

### Synthesis of E2 to E5. (a) Isopropanol/HFIP or Isopropanol, Heating, 64-480h

### Example E2

HFIP (252 mg, 1.5 mmol, 3 eq) was added to a stirring solution of N,N-bis(3-aminopropyl)methylamine (65.6 mg, 0.5 mmol, 1 eq) and tert-butyl (3-(N-methyl-acryl-amido)propyl)carbamate (1.21 g, 5 mmol, 10 eq) in propan-2-ol. The reaction mixture was heated to 60° C. for 64 h. The mixture was then cooled down and concentrated in vacuo. The crude viscous oil was purified using silica column chromatography eluting with mixtures of MeOH/CHCl₃ (0-20%) yielding E2 as a viscous oil.

HPLC-ESI-MS: m/z Calc. for C₅₅H₁₀₇N₁₁O₁₂ 1113.81. Measured [M+H]+ 1114.83, [M+H]²+558.08, [M+H]²⁺−Boc 508.00 [M+H]²⁺−2×Boc 458.00, [M+H]²⁺−3×Boc 408.00, [M+H]²⁺−4×Boc 357.92.

### Example E3

Di-n-propylene-triamine (65.6 mg, 0.5 mmol, 1 eq) and tert-butyl (3-(N-methylacrylamido)propyl)carbamate (1.21 g, 5 mmol, 10 eq) were dissolved in propan-2-ol and HFIP (1 mL). The reaction mixture was heated to 60° C. for 480 h. Although the reaction was not completed after 480h the reaction mixture was cooled down and concentrated in vacuo. The crude viscous oil was purified using silica column chromatography eluting with mixtures of MeOH/CHCl₃ (0-20%) yielding E3 as a viscous oil.

ESI-MS: m/z Calc. for C₆₆H₁₂₇N₁₃O₁₅ 1341.96. Measured [M+H]⁺ 1343.00, [M+Na]+1364.92, [M+H]²+672.08, [M+H]²⁺−Boc 622.08 [M+H]²⁺−2×Boc 572.08, [M+H]²⁺−3×Boc 522.08, [M+H]²⁺−4×Boc 472.00, [M+H]²⁺−5×Boc 472.00.

### Example E4

Diethylenetriamine (103 mg, 1 mmol, 1 eq) and tert-butyl (3-(N-methylacrylamido)propyl)carbamate (2.42 g, 10 mmol, 10 eq) were dissolved in propan-2-ol, and heated to reflux. Although after 305h at reflux, the reaction was still not completed, the reaction was stopped at that point. The reaction mixture was concentrated in vacuo giving a viscous amber oil. The crude product E4 was purified using silica column chromatography, using mixtures of MeOH/CHCl3 (0-20%).

### Example E5

In a flask of 5 ml, 1,4-diaminobutane (88 mg, 1 mmol, 1 eq) and tert-butyl (3-(N-methylacrylamido)propyl)carbamate (1.21 g, 5 mmol, 5 eq) were dissolved in propan-2-ol (2 ml). The reaction mixture was heated to 55° C. After 350h the reaction was almost complete. The reaction mixture was concentrated in vacuo. The crude product was purified using silica column chromatography, eluting with mixtures of CHCl₃/MeOH (0-20%) to obtain E5 as an amber viscous oil (756 mg, 0.72 mmol, 72%).

¹H NMR (400 MHz, Chloroform-d) δ 5.81-5.20 (m, 4H), 3.43 (t, J=6.4 Hz, 6H), 3.36 (t, J=7.8 Hz, 2H), 3.16 (q, J=6.4 Hz, 2H), 3.06 (q, J=6.3 Hz, 6H), 3.02 (s, 9H), 2.91 (s, 3H), 2.81 (t, J=7.3 Hz, 8H), 2.48 (m, 12H), 1.80 (p, J=6.9 Hz, 2H), 1.66 (p, J=6.4 Hz, 6H), 1.43 (m, 40H). ¹³C NMR (101 MHz, CDCl₃) δ 172.4, 171.6, 156.2, 156.1, 79.0, 78.8, 77.5, 77.4, 77.2, 76.8, 54.1, 49.5, 49.4, 47.6, 44.6, 44.5, 38.0, 36.9, 35.4, 35.3, 33.3, 30.9, 30.4, 29.1, 28.4, 28.4, 27.2, 24.8. ESI-MS: m/z Calc. for C₅₂H₁₀₀N₁₀O₁₂ 1056.75. Measured [M+H]⁺ 1057.92, [M+Na]+1079.92, [M+H]²+529.58, [M+H]²⁺−Boc 479.50 [M+H]²⁺−2×Boc 429.50, [M+H]²⁺−3×Boc 379.50.

### F. The Functionalization of Primary Amines with an Acrylate-Ester, Followed by Functionalization of the Remaining Secondary Amines with an Acryl-Amide (Michael-Additions). Amides can Also be Reacted First, and Esters Second

### General Example F1: G1-ACR-nC14-4/AmAlc-4

This general example describes the subsequent Michael-addition reactions of G1-PPI—(NH₂)₄ dendrimer with n-tetradecyl acrylate and N-(2-hydroxyethyl)-N-methyl acrylamide, leading to PPI-dendrimer with 4 n-tetradecyl-ester and 4 N-(2-hydroxyethyl)-N-methyl amide groups.

PPI dendrimer G1 (0.10 g, 0.315 mmol) was dissolved in iso-propanol (1 mL) and tetradecyl acrylate (0.339 g, 4 moleqs per dendrimer) was added. The mixture was stirred at an oil bath temperature of 55° C. for 24 hours which resulted in full conversion of the primary amines. A slight excess of acrylate reactant was still present. According to ¹H-NMR the acrylate had reacted selectively with the primary amines, resulting in mono functionalization of all primary amine groups. Next, N-(2-hydroxyethyl)-N-methyl acrylamide (0.204 g, 1.57 mmol, 5 moleqs) was added. The mixture was stirred at 55° C. for a total of 6 days. Unreacted acrylate or acrylamide was then removed by stirring the reaction mixture with amine-functionalized silica scavenger. The mixture was filtered and the silica scavenger was washed with chloroform/MeOH (5%). The filtrate was evaporated to dryness, giving oil product that did not contain any acrylate or acrylamide anymore. Yield: 344 mg (50%; this lower yield could be due to the dendrimer sticking in part to the silica scavenger). The ¹H-NMR spectrum was in agreement with the desired structure.

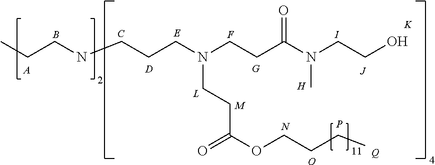

¹H NMR (399 MHz, Chloroform-d) δ 4.05 (t, J=6.8 Hz, 8H, N), 3.72 (q, J=5.8 Hz, 8H, J), 3.49 (dt, J=17.9, 4.7 Hz, 8H, I), 3.12-2.94 (singlets, 12H, H), 2.86 (t, J=3.2 Hz, 1H), 2.78 (q, J=7.5 Hz, 16H, F, L)), 2.68-2.26 (m, 36H, B, C, E, G, M), 1.60 (td, J=13.7, 7.0 Hz, 16H, D, O), 1.26 (s, 92H, A, P), 0.88 (t, J=6.8 Hz, 12H, K).

MALDI-TOF-MS (CHCA matrix, positive reflector mode): Obs. m/z=(M+H)⁺ 1907.62, (M+Na)⁺ 1929.6. Calculated: C₁₀₈H₂₁₂N₁₀O₁₆ (exact mass 1905.61; molecular weight 1906.94). Small peak also observed at m/z=2046.78 (5 tetradecyl acrylate and 3 acrylamide groups).

### G. The Functionalization of Amines with an Epoxide

### General Example G1: G1-EPX-nC8-8

This general example describes the reaction of G1-PPI—(NH₂)₄ dendrimer with (1,2-epoxy)-n-decane leading to PPI-dendrimer with 2-hydroxy C10 groups.

PPI dendrimer G1 (0.10 gram 0.315 mmol) was dissolved in iso-propanol (1 mL) and 1,2 epoxy-decane (0.43 g, 8.8 moleqs) was added. The reaction mixture was stirred in a closed vial at 90° C. under an inert atmosphere of nitrogen, and was then evaporated to dryness. The residue was stirred in MeCN and the suspension was put at −20° C. for several hours. The supernatant was decanted. This procedure was repeated two times, after which the residue did not contain any remaining 1,2-epoxydecane anymore. The residue was dried in vacuo, giving the product. Yield: 390 mg (80%). The ¹H-NMR spectrum was in agreement with the desired structure.

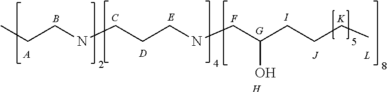

¹H NMR (399 MHz, Methanol-d4) δ 3.62 (tt, J=7.2, 3.8 Hz, 8H, G), 2.68-2.29 (m, 36H, B, C, E, F), 1.63 (p, J=7.1 Hz, 8H, D), 1.59-1.12 (m, 112H, I, J, K), 1.11-0.67 (m, 24H, L).

MALDI-TOF-MS (CHCA matrix, positive reflector mode): Obs. m/z=(M+H)⁺ 1567.57, (M+Na)⁺ 1589.56, (M+K)⁺ 1605.53. Calculated: C₉₆H₂₀₀N₆O₈ (exact mass 1565.54; molecular weight 1566.69).

### Example G2. G1-EPX-nC10-8

The reaction between G1-PPI—(NH₂)₄ (n-butylene core) and 1,2-epoxy-dodecane was performed in a similar way as Example G1. Yield: 447 mg (79%). The ¹H-NMR spectrum was in agreement with the desired structure. MALDI-TOF-MS (CHCA matrix, positive reflector mode): Obs. m/z=(M+H)⁺ 1791.83, (M+Na)⁺ 1813.82, (M+K)⁺ 1829.79. Calculated: C₁₁₂H₂₃₂N₆O₈ (exact mass 1789.79; molecular weight 1791.12).

### Example G3. G2-EPX-nC10-16

The reaction between G2-PPI—(NH₂)₈ (n-butylene core) and 1,2-epoxy-dodecane was performed in a similar way as Example G1. Yield: 697 mg (84%). The ¹H-NMR spectrum was in agreement with the desired structure. MALDI-TOF-MS (CHCA matrix, positive reflector mode): Obs. m/z=(M+H)⁺ 3722.78, (M+Na)⁺ 3744.75, (M+K)⁺ 3760.74. Calculated: C₂₃₂H₄₃₀N₁₄O₁₆ (exact mass 3719.72; molecular weight 3722.47).

### Example G4. G3-EPX-nC10-32

The reaction between G3-PPI—(NH₂)₁₆ (n-butylene core) and 1,2-epoxy-dodecane was performed in a similar way as Example G1. Yield: 413 mg (88%). The ¹H-NMR was in agreement with the desired structure.

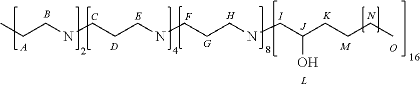

¹H NMR (399 MHz, Chloroform-d) δ 4.97-4.07 (m, 16H, L), 3.59 (s, 16H, J), 2.90-2.20 (m, 84H, B, C, E, F, H, I), 1.79-1.08 (m, 316H, A, D, G, K M, N), 0.88 (t, J=6.8 Hz, 48H, O).

### Example G5. G1(PPeI)-EPX-nC10-8

G1-PPeI (100 mg, 0.23 mmol, 1 eq) and 1,2-epoxydodecane (0.81 mL, 684 mg, 3.72 mmol, 16 eq) were dissolved in propan-2-ol (IPA; 1 mL). The mixture was heated to 60° C. for 16h (monitoring with ¹H-NMR). The mixture was cooled down and concentrated in vacuo. The viscous oil was stirred in MeCN, cooled down to −20° C. and allowed to settle on the bottom of the flask. The supernatant was decanted, and the procedure was repeated three times to give the product as a viscous oil (344 mg, 0.20 mol, 88%).

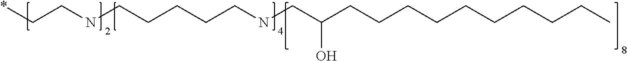

¹H NMR (399 MHz, Chloroform-d) δ 3.60 (m, 8H), 3.19 (m, 8H), 2.64-2.47 (m, 10H), 2.38 (p, J=7.8, 6.9 Hz, 26H), 1.65-1.16 (m, 172H), 0.88 (t, J=6.7 Hz, 24H). MALDI-TOF-MS (CHCA matrix, positive reflector mode): Obs. m/z=1903.99 (M+H)⁺, 1925.92 (M+Na)⁺ Calculated: C₁₂₀H₂₄₈N₆O₈ (exact mass 1901.92; molecular weight 1903.34).

### Example G6. G2(PAMAM-C2)-EPX-nC8-16

1,2-Epoxydecane (700 mg, 4.38 mmol, 32 eq) was added to a stirred solution of G2-PAMAM-C2 (ethylene diamine core, 8 amine end groups; 200 mg, 0.14 mmol) in propan-2-ol (1 mL). The reaction mixture was heated to 60° C. for 88 h, and was then concentrated in vacuo. The residue was stirred in MeCN and cooled down to −20° C., allowing the product to settle at the bottom of the flask. The supernatant was decanted and this trituration procedure was repeated three times yielding a yellow viscous oil (422 mg, 107 μmol, 77%).

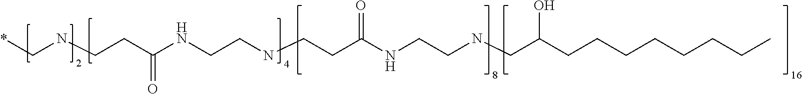

¹H NMR (399 MHz, Chloroform-d) δ 7.92 (s, 12H), 4.60 (d, J=29.0 Hz, 16H), 3.59 (s, 16H), 3.31 (t, J=41.3 Hz, 24H), 2.99-2.09 (m, 108H), 1.62-1.06 (m, 224H), 0.88 (t, J=6.7 Hz, 48H). MALDI-TOF-MS (DCBT matrix, positive reflector mode): Obs. m/z=(M+H)⁺ 3930.44. Calculated: C₂₂₂H₄₄₈N₂₆O₂₈ (exact mass 3927.44; molecular weight 3930.18).

### Example G7. Bis(3-aminopropyl)amine-EPX-C8-5

1,2-Epoxydecane (1.56 g, about 10 mmol, 10 eq) was added to a stirred solution of bis(3-aminopropyl)amine (131 mg, 1 mmol) in IPA (1 mL). The reaction mixture was heated to 60° C. for 40h, and was then concentrated in vacuo. The residue was stirred in MeCN and cooled down to −20° C., allowing the product to phase separate from the MeCN solvent. The supernatant was carefully removed using a pipette, and the trituration procedure was repeated three times. The residue was dried in vacuo yielding a clear colorless oil (950 mg).

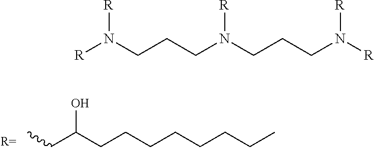

¹H NMR (399 MHz, Methanol-d₄) δ 7.89 (s, 1H), 3.63 (qd, J=7.2, 4.5, 3.6 Hz, 5H), 2.74-2.26 (m, 17H), 1.64 (q, J=7.3 Hz, 4H), 1.56-1.21 (m, 70H), 1.00-0.79 (m, 15H). MALDI-TOF-MS (ODCB matrix, positive reflector mode): Obs. m/z=(M+H)⁺ 912.87. Calculated: C₅₆H₁₁₇N₃O₅ (exact mass 911.90; molecular weight 912.57).

### Example G8. Bis(2-amino-ethyl)amine-EPX-C8-5

Bis(2-aminoethyl)amine (103 mg, 1 mmol) and 1,2-epoxydecane (1.56 g, about 10 mmol, 10 eq) were reacted in IPA (1 mL). A similar procedure was employed as described for the Example G7 material. Yield: 958 mg 1.08 mmol.

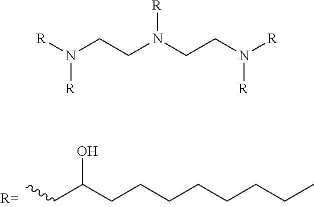

¹H NMR (399 MHz, Methanol-d₄) δ 3.83-3.55 (m, 5H), 2.91-2.29 (m, 18H), 1.61-1.10 (m, 70H), 1.02-0.79 (m, 15H). MALDI-TOF-MS (DCTB matrix, positive reflector mode): Obs. m/z=(M+H)⁺ 884.84, [M+Na]+906.82. Calculated: C₅₄H₁₁₃N₃O₅ (exact mass 883.87; molecular weight 884.51).

### Example G9. Bis(3-aminopropyl)methylamine-EPX-C8-4

1,2-Epoxydecane (269 mg, 1.68 mmol, about 6 eq) was added to a stirred solution of N,N-bis(3-aminopropyl)methylamine (42 mg, 0.29 mmol) in IPA (1 mL). The reaction mixture was heated to 60° C. for 16h, and was then concentrated in vacuo. The residue was stirred in MeCN and was cooled down to −20° C., allowing the product to phase separate from the MeCN solvent. The supernatant was carefully removed using a pipette and the trituration procedure was repeated three times. The residue was dried in vacuo yielding a clear colorless oil (170 mg, 0.22 mmol, 77%).

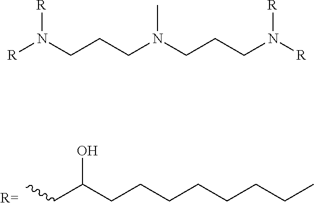

¹H NMR (400 MHz, Chloroform-d) δ 4.93-3.84 (m, 3H), 3.84-3.52 (m, 4H), 3.46 (s, 2H), 2.89-2.53 (m, 4H), 2.53-2.20 (m, 11H), 2.20-2.10 (m, 2H), 1.62 (dt, J=12.7, 6.4 Hz, 4H), 1.53-1.16 (m, 56H), 0.88 (t, J=6.7 Hz, 12H). MALDI-TOF-MS (CHCA matrix, positive reflector mode): Obs. m/z=(M+H)⁺ 770.73, Calculated: C₄₇H₉₉N₃O₄ (exact mass 769.76; molecular weight 770.33).

### H. The Functionalization of Primary Amines with an Acrylate-Ester, Followed by Alkylation of the Remaining Secondary Amines

### Example H1. G1-ACR-nC14-4/CH₃-4

This general example describes the Michael-addition reaction of G1-PPI—(NH₂)4 dendrimer with n-tetradecyl acrylate, followed by reaction with paraformaldehyde, leading to a PPI-dendrimer with 4 n-tetradecyl-ester groups and 4 methyl groups.

G1 PPI dendrimer DAB-Am-4 (0.21 g, 0.671 mmol) was dissolved in isopropanol (1 mL), and tetradecyl acrylate (0.72 g, 2.68 mmol, 4 moleqs) was added. The mixture was stirred in a closed vial at 55° C. under an inert atmosphere of nitrogen for 24 hours which resulted in full conversion of the primary amines. Almost exclusive mono-functionalization of each of the primary amine groups is observed. The reaction mixture is evaporated to remove the isopropanol and the residue is co-evaporated three times with chloroform. The residue is dissolved in formic acid (2 mL). Para-formaldehyde (121 mg, 4.0 mmol, 6 moleqs) is added, as well as THF (1 mL) for solubility. The mixture was stirred at 60° C. The progress of the reaction was monitored with ¹H-NMR. After 3.5 hours the reaction was complete, and the mixture was evaporated to dryness and co-evaporated 2 times with chloroform. The crude mixture was dissolved in chloroform and washed with 0.1 M NaOH (aq) followed by saturated NaCl (aq) and dried with Na₂SO₄. The crude product was stirred in MeCN and was put at 0° C. for a few hours to induce full phase separation. Subsequently, the supernatant was decanted. This procedure was repeated two times. The residue was put over a silica column starting with 5% MeOH/chloroform to remove impurities. The product is finally eluted from the column by changing the eluent to 5% MeOH and 1% TEA/chloroform. Yield: 560 mg (58%). The ¹H-NMR spectrum was in agreement with the desired structure.

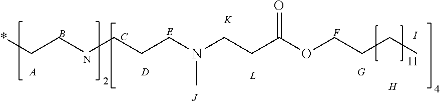

¹H NMR (399 MHz, Chloroform-d) δ 4.06 (td, J=6.8, 1.3 Hz, 8H, F), 2.69 (t, J=7.3 Hz, 8H, K), 2.62-2.29 (m, 25H, B, C, E, L), 2.23 (d, J=1.3 Hz, 12H, J), 1.81-1.55 (m, 14H, D, G), 1.55-1.01 (m, 90H, H), 0.99-0.73 (m, 12H, I). MALDI-TOF-MS (CHCA matrix, positive reflector mode): Obs. m/z=(M+H)⁺ 1446.37, Small peak with m/z=M+H)⁺, 1701.59 (5 C14-chains and 3 CH₃). Calculated: C₃₃H₁₇₆N₆O₈ (exact mass 1445.35; molecular weight 1446.41).

### J. The Amidation of Amines, Optionally Followed by Alkylation of Interior Tertiary Amines

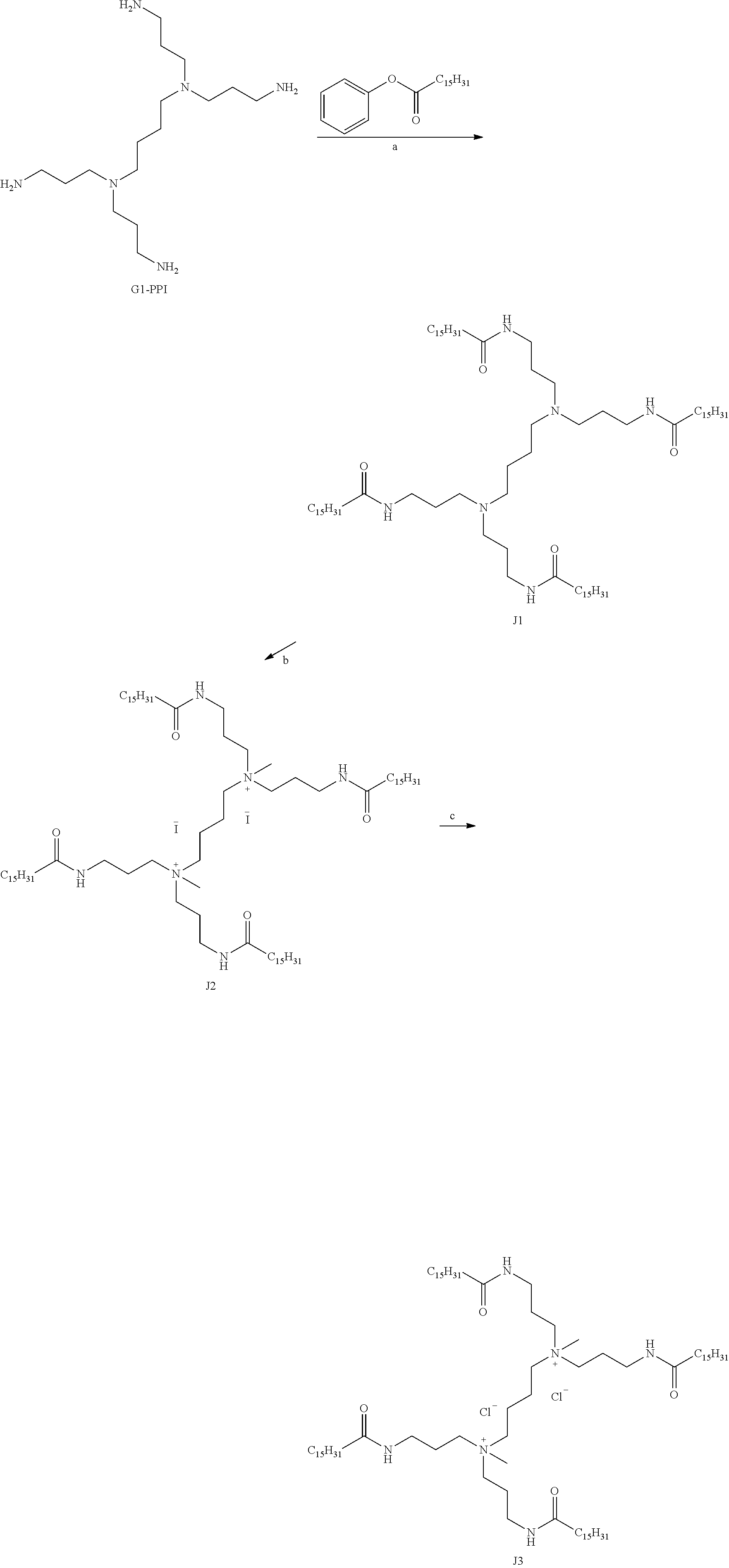

Synthesis of G1 PPI dendrimer amides, also with internal quaternization. (a) toluene, TEA, 90° C., 6h; (b) Mel, chloroform/MeOH, 50° C.; (c) Amberlite IRA 400 (Cl⁻).

### Example J1. G1-amide-nC16-4

N,N,N,N-((Butane-1,4-diylbis(azanetriyl))tetrakis(propane-3,1-diyl))tetrapalmitamide. N,N′-(Butane-1,4-diyl)bis(N1-(3-aminopropyl)propane-1,3-diamine) (PPI-G1, 1 g) was co-evaporated twice with a sufficient amount of toluene. Then, dissolved in toluene (4 mL), it was added to phenyl palmitate (6.3 g) in toluene (16 mL). Triethylamine (9 mL) was added and the mixture was stirred at 90° C. for 6 hours. The reaction mixture was concentrated in vacuo and the resulting waxy solid was triturated in acetonitrile (80 mL) for 3 hours. After filtrating and washing with acetonitrile the residue was dried and triturated in hexane. Filtration, washing with hexane and drying of the residue yielded 3.3 g (82%) of the pure product as an off-white solid. The ¹H-NMR spectrum was in agreement with the desired structure.

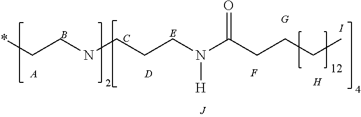

¹H NMR (399 MHz, Chloroform-d) δ 6.48 (t, J=5.7 Hz, 3H, J), 3.29 (q, J=6.5 Hz, 8H, E), 2.52-2.26 (m, 12H, B, C), 2.16 (t, J=7.7 Hz, 8H, F), 1.61 (dd, J=15.4, 8.6 Hz, 22H, A, D, G), 1.25 (s, 95H, H), 0.88 (t, J=6.7 Hz, 12H, 1). MALDI-TOF-MS (CHCA matrix, positive reflector mode): Obs. m/z=(M+H)⁺ 1270.31. Small peak with m/z=M+Na⁺, 1292.23. Calculated: C₃₀H₁₆₀N₆O₄ (exact mass 1269.25; molecular weight 1270.20).

### Example J2. G1-amide-nC16-4 (2 Mel)

N,N,N,N-((Butane-1,4-diylbis(azanetriyl))tetrakis(propane-3,1-diyl))tetrapalmitamide (2 Mel ammonium adduct). Example J1 (4 g) was stirred in a 1:1 chloroform/methanol mixture (80 mL) at 45° C. To this turbid mixture was added methyl iodide (3.14 mL). Raising the oil bath temperature to 50° C. and adding 25 mL of chloroform resulted in a clear mixture that was stirred for 16 hours. After adding an extra amount of methyl iodide (1 mL) stirring was continued for 4 hours. The mixture was then concentrated in vacuo and co-evaporated twice with methanol and once with chloroform. To get full methylation the product was again dissolved in chloroform/methanol and with 10 equivalents of methyl iodide and stirred for 1 hour at 90° C. in a capped vial under microwave irradiation. Concentrating the mixture in vacuo and co-evaporating with chloroform yielded the off white product quantitatively. The ¹H-NMR spectrum was in agreement with the desired structure.

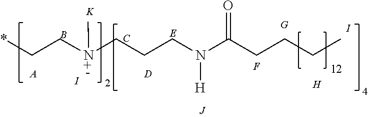

¹H NMR (400 MHz, Chloroform-d) δ 7.41 (t, J=6.1 Hz, 2H), 7.35 (s, 4H), 3.53 (s, 3H), 3.37 (p, J=1.6 Hz, 2H), 3.31 (p, J=5.7 Hz, 15H, C, E), 3.12 (s, 9H), 3.04 (s, 6H, K), 2.23 (t, J=7.6 Hz, 8H, F), 2.14-1.81 (m, 12H, A, D), 1.61 (p, J=7.2 Hz, 8H, G), 1.26 (s, 99H, H), 0.88 (t, J=6.7 Hz, 13H, 1).

### Example J3. G1-amide-nC16-4 (2 MeCl)

N,N,N,N-((Butane-1,4-diylbis(azanetriyl))tetrakis(propane-3,1-diyl))tetrapalmitamide (2 MeCl ammonium adduct). Example J2 (3 g) was dissolved in 150 mL of a chloroform/methanol mixture (2:1) to acquire a clear solution. Ion exchange resin (50 gram) Amberlite IRA 400 (Cl⁻) was stirred in water and poured on a 1 cm diameter column. The column was flushed with water and thereafter with methanol. Slowly the amount of chloroform in the flushing eluent was raised to 66%.

To avoid the resin beads to float a plug of wadding was placed on top of the resin. The solution of J2 was eluted over the resin with a chloroform/methanol mixture (2:1). All collected eluent was concentrated to give the desired product (2.4 g). The ¹H-NMR spectrum was in agreement with the desired structure. To show that all iodide ions were replaced, an organic solution of the product was extracted with a 1M NaBr solution. The aqueous layer was then used for a starch test which showed no coloration proving full exchange of the iodide anions. As a reference benchmark, iodide containing dendrimer solutions were tested as well. These solutions became blue.

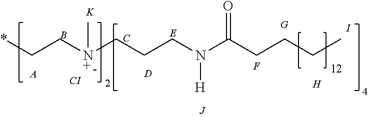

¹H NMR (400 MHz, CDCl₃) δ 3.45 (m, 12H, B, C), 3.37 (t, 8H, E), 3.06 (s, 6H, K), 2.41 (t, 8H, F), 2.15 (m, 8H, D), 1.96 (m, 4H, A), 1.62 (m, 8H, G), 1.25 (m, 96H, H), 0.88 (t, 12H, 1).

### Example J4. G3-amide-nC16-16

This material J4 was obtained by reacting PPI-dendrimer G3 (DAB-Am-16) with phenyl palmitate, and was performed in a similar way as done for Example J1. Yield: 5.88 g (90%). The ¹H-NMR spectrum was in agreement with the desired structure.

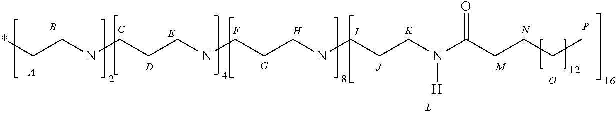

¹H NMR (400 MHz, Chloroform-d) δ 11.04 (d, J=207.4 Hz, 8H, NH), 7.60-7.16 (m, 9H), 4.06-2.63 (m, 113H, B, C, E, F, H, I, K), 2.46-2.08 (m, 54H, D, G, M), 2.08-1.70 (m, 36H, A, J), 1.57 (p, J=7.2, 6.7 Hz, 33H, N), 1.26 (d, J=9.1 Hz, 387H, O), 0.88 (t, J=6.7 Hz, 48H, P). MALDI-TOF-MS (DCTB matrix, positive reflector mode): Obs. m/z=(M+H)⁺ 5503.04. Calculated: C₃₄₄H₆₈₈N₃₀O₁₆ (exact mass 5497.39; molecular weight 5501.48).

### Example J5. G3-amide-nC16-16 (14 Mel)

This material J5 was obtained by reacting J4 with Mel, and was performed in a similar way as done for Example J2. Yield: 1.32 g (97%).The ¹H-NMR spectrum was in agreement with the desired structure.

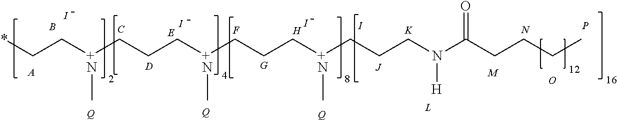

¹H NMR (400 MHz, CDCl₃/CD₃OD) δ 7.54 (d, J=16.5 Hz, 1H), 7.30 (s, 9H), 3.84 (d, J=61.2 Hz, 48H), 3.55 (d, J=35.9 Hz, 41H), 3.44 (s, 5H), 3.43-3.12 (m, 60H), 2.59 (s, 23H), 2.40 (s, 35H), 2.25 (t, J=7.8 Hz, 35H), 2.06 (d, J=10.4 Hz, 33H, M), 1.60 (p, J=7.2 Hz, 33H, N), 1.51-1.00 (m, 387H, O), 1.00-0.76 (m, 48H, P).

### Example J6. G3-amide-nC16-16 (14 MeCl)

This material J6 was obtained by eluting J5 over an ion exchange column, and was performed in a similar way as done for Example J3. Yield: 2.55 g (99%).The ¹H-NMR spectrum was in agreement with the desired structure.

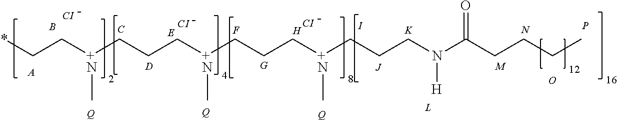

¹H NMR (400 MHz, CDCl₃/CD₃OD) δ 7.87 (s, 12H), 7.30 (t, J=7.4 Hz, 11H), 4.16-2.94 (m, 145H), 2.94-2.61 (m, 52H), 2.46 (s, 22H), 2.20 (dq, J=17.3, 8.2 Hz, 30H), 1.96 (s, 33H), 1.60 (s, 28H), 1.25 (dt, J=20.6, 9.1 Hz, 354H, O), 0.88 (dt, J=16.5, 7.5 Hz, 48H, p).

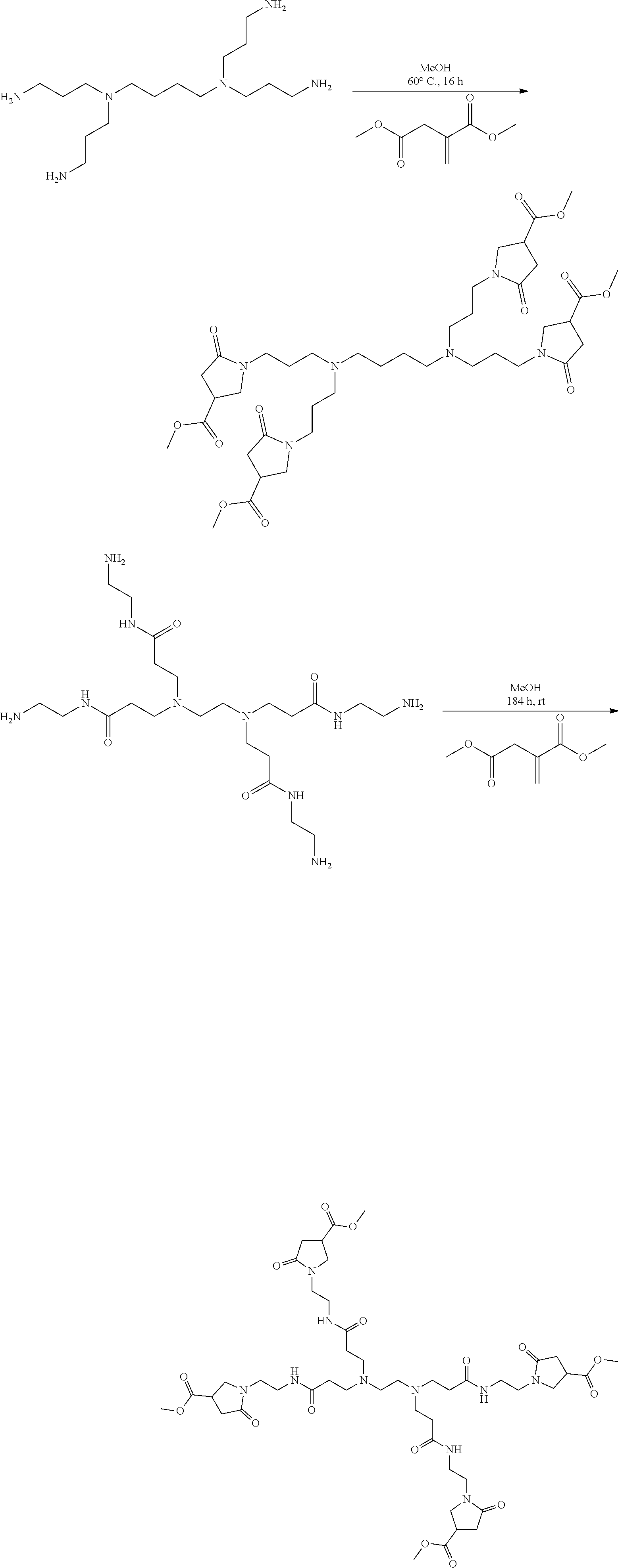

### Synthesis of Dendrimers G1-Dimethylitaconate-4 and G1(PAMAM-C2)-Dimethylitaconate-4. Transesterification (by Longer Chain Alcohols) or Amidation (by Longer Chain Primary or Secondary Amines) of the 4 Methyl-Ester End Groups Will Afford More Apolar Structures

### Example J7. G1-Dimethylitaconate-4

Dimethyl itaconate (0.66 g, 4.2 mmol, 4.2 eq) was added to a stirred solution of G1-PPI—(NH₂)₄ (316 mg, 1 mmol) in IPA (2 mL) was. The reaction mixture was heated to 60° C. for 16h, and was then poured into water (15 mL). The water layer was washed with diethyl ether (4×5 mL). Traces of solvent were removed in vacuo at room temperature. The water layer was lyophilized giving an amber oil. The product was purified by silica column chromatography using MeOH/CHCl₃ (20-30% MeOH) eluents. Yield: a colorless oil (414 mg, 0.50 mmol, 50%).

¹H NMR (400 MHz, Chloroform-d) δ 3.74 (s, 12H), 3.70-3.53 (m, 8H), 3.39-3.17 (m, 12H), 2.77-2.60 (m, 8H), 2.53-2.27 (m, 12H), 1.66 (m, 8H), 1.39 (m, 4H). ESI-MS: m/z Calc. for C₄₀H₆₄N₆O₁₂ 820.46; Obs. [M+H]²+411.50, [M+H]⁺ 820.50, [M+Na]+853.50.

### Example J8. G1(PAMAM-C2)-Dimethylitaconate-4

Dimethylitaconate (514 mg, 3.25 mmol, 4.9 eq) was added to a solution of G1-PAMAM-C2 (ethylene diamine core, 4 amine end groups; about 340 mg, 0.66 mmol) in MeOH (2 mL). The mixture was stirred at room temperature and the reaction progress was monitored using HPLC-MS. After 184h the reaction was completed. The mixture was poured into water (15 mL), and the water layer was washed with diethyl ether (4×5 mL). Traces of solvent were removed in vacuo and the water layer was lyophilized to yield a semi-solid colorless wax (834 mg) that still contained some ether.

¹H NMR (400 MHz, Chloroform-d) δ 7.71 (t, J=5.3 Hz, 4H), 3.74 (s, 16H), 3.66 (dd, J=9.9, 5.9 Hz, 4H), 3.55-3.39 (m, 10H), 3.39-3.17 (m, 12H), 2.74-2.55 (m, 16H), 2.52 (s, 4H), 2.32 (t, J=6.5 Hz, 8H). ESI-MS: m/z Calc. for C₄₆H₇₂N₁₀O₁₆ 1021.14; Obs. [M+H]²+511.58, [M+H]⁺ 1021.50, [M+Na]+1043.50.

### Example J9. G1-Lysine-N²-Oleate-4

G1 PPI dendrimer (35 mg; 0.11 mmol; 0.44 mmol primary amine groups) and triethylamine (200 μL) were dissolved in DCM (9 mL) and stirred at room temperature in a nitrogen atmosphere. 2,3,5,6-Tetrafluorophenyl N⁶-(tert-butoxy-carbonyl)-N²-oleoyl-L-lysinate (292 mg, 0.44 mmol, 4.00 eq) was added and the solution was stirred for 24h. Chloroform (50 mL) was added and the organic phase was washed with aqueous 1 N NaOH (20 mL), followed by water (20 mL) and brine (20 mL). The solution was co-evaporated with chloroform (3×20 mL) to remove triethylamine. Yield: 209 mg of a colorless oil. The oil was dissolved in DCM (10 mL) and TFA (1 mL) and the solution was stirred for 20 h at room temperature in a nitrogen atmosphere. The mixture was evaporated till dryness, and the residue was dissolved in CHCl₃ (70 mL). The solution was washed with aqueous 1N NaOH (20 mL) followed by brine (20 mL). The solution was evaporated till dryness and a white wax product was obtained. Yield: 180 mg (86%).

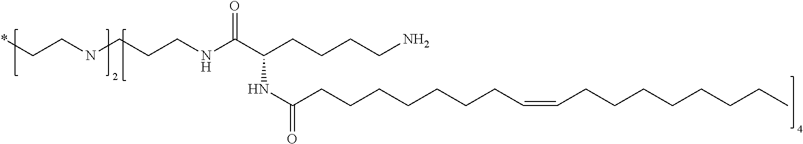

¹H NMR (400 MHz, Chloroform-d) δ 7.98 (s, 4H), 7.25-7.00 (s, 4H), 5.38-5.28 (m, 8H), 4.45 (s, 4H), 3.50-2.60 (m, 32H), 2.48 (s, 8H), 2.19 (s, 8H), 2.00 (m, 16H), 1.85-1.20 (m, 120H), 0.88 (t, J=6.9 Hz, 12H). FTIR (cm⁻¹) 3282 (m), 2924 (s), 2854 (m), 1692 (m), 1637 (vs), 1534 (m), 1455 (m), 1365 (m), 1248 (m), 1171 (s). MALDI-TOF-MS (CHCA matrix, positive reflector mode): Obs. m/z=(M+Na)⁺ 1909.66 (2^{nd} isotope peak). Calculated: C₁₁₂H₂₁₆N₁₄O₈ (exact mass 1885.69; molecular weight 1887.00).

### Example J10. G1-Lysine-N⁶-Oleate-4

A solution of G1 PPI dendrimer (31.0 mg, 0.098 mmol; 0.39 mmol primary amine groups) in DCM (1 mL) was added to a solution of 2,3,5,6-tetrafluorophenyl N²-(tert-butoxy-carbonyl)-N⁶-oleoyl-L-lysinate (282 mg, 0.43 mmol) and triethylamine (100 μL) in DCM (3 mL) in a nitrogen atmosphere. After 30h an ¹H-NMR analysis showed complete conversion. To remove the excess of the TFP-ester, Biotage MP-Trisamine resin (125 mg) was added and stirring continued for 30h. DCM (50 mL) was added and the mixture was filtered. The filtrate was washed with 1N NaOH (20 mL), water (20 mL) and brine (20 mL). After removal of the solvent the dendrimer was obtained as a white solid. Yield: 198 mg. The dendrimer (195 mg) was dissolved in DCM (4 mL) in a nitrogen atmosphere, and TFA (1 mL) was added. After 23 h the reaction mixture was concentrated on a rotary evaporator. The product was co-evaporated three times with toluene to remove excess TFA, and was then dissolved in chloroform (50 mL) and shaken with 1N NaOH (2 mL) followed by brine (10 mL). Removal of the solvent yielded a colorless oil. Yield: 180 mg (97%). The 1H-NMR spectrum was in agreement with the desired structure.

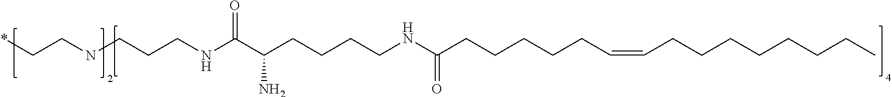

¹H NMR (400 MHz, Chloroform-d) δ 7.63 (s, 4H), 6.16 (t, 4H), 5.38-5.29 (m, 8H), 4.16 (s, 4H), 3.35-3.20 (m, 16H), 2.41 (t, 4H), 2.36 (t, 4H), 2.15 (t, J=9.7 Hz, 8H), 2.0 (m, 16H), 1.9-1.0 (m, 136H), 0.87 (t, J=6.9 Hz, 12H). FTIR (cm⁻¹) 3309 (s), 2923 (vs), 2854 (s), 1639 (vs), 1549 (s), 1466 (w). MALDI-TOF-MS (CHCA matrix, positive reflector mode): Obs. m/z=(M+Na)⁺ 1909.68 (2^{nd} isotope mass). Calculated: C₁₁₂H₂₁₆N₁₄O₈ (exact mass: 1885.69; molecular weight 1887.00).

### Example J11. G1-Arginine-Oleate-4

A solution of G1 PPI dendrimer (69 mg, 0.22 mmol, 0.87 mmol primary amine groups) in THF (2 mL) was added to oleoyl-L-arginine (463 mg, 0.97 mmol), followed by the addition of chloroform (2 mL). After stirring for several minutes, a gel-like mixture had formed. HFIP (100 μL) was added, and stirring continued at 40° C. resulted in the formation of a clear solution. After cooling to room temperature, DCC (399 mg, 1.93 mmol) was added. The resulting clear solution was heated to 40° C. and it turned turbid white (due to DCU formation). The reaction progress was monitored by MALDI-TOF-MS analyses of samples. After 6 days of reaction, more oleoyl-L-arginine (220 mg, 0.46 mmol) and DCC (156 mg, 0.76 mmol) were added. Three days later MALDI-TOF-MS indicated full conversion of the dendrimer and the reaction was stopped. Water (0.38 mL) and acetic acid (0.20 mL) were added. Concentration on a rotary evaporator resulted in 1.10 g of crude product.

Water (10 mL) was added to a part (500 mg) of the crude product, and the mixture was sonicated for 30 minutes. After standing overnight, the nearly clear water solution was separated from the white precipitate (mainly DCU) and was transferred to a dialyses tubing (CE 500-1000 Dalton). The solution was dialyzed versus water (1 L) containing acetic acid (2 mL), followed by water (1 L) with acetic acid (1 mL), followed by water (1 L) with 1N NaOH (2 mL), and finally with water (1 L). After lyophilization a white fluffy powder was obtained. Yield: 188 mg. The ¹H-NMR spectrum was in agreement with the desired structure.

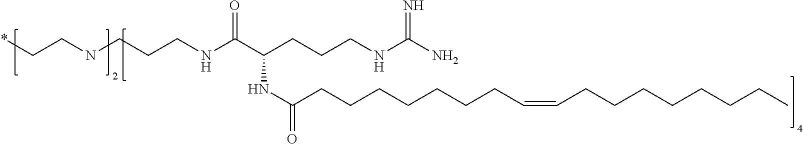

¹H NMR (400 MHz, Chloroform-d) δ 8.20 (s, 4H), 7.60-7.00 (broad peak, 20H), 5.38-5.28 (m, 8H), 4.40-4.10 (m, 4H), 3.30-3.10 (m, 16H), 2.50-1.10 (m, 152H), 0.88 (t, J=6.8 Hz, 12H). FTIR (cm⁻¹) 3280 (m), 3184 (m), 2984 (vs), 2854 (vs), 1644 (vs), 1549 (vs), 1456 (m), 1403 (m). MALDI-TOF-MS (CHCA matrix, positive reflector mode): Obs. m/z=(M+H)⁺ 1999.73 (2^{nd} isotope peak). Calculated C₁₁₂H₂₁₆N₂₂O₈ (1998.72 for 22^{nd} isotope peak); molecular weight 1999.06.

### K. Functionalization of Primary Amines with Acryl-Esters Followed by Acylation of the Remaining Secondary Amines (Here, the Products are Mixtures of Polyvalent Molecules)

### Example K1. G1 PPI Dendrimer Modified with n-Octyl-Acrylate and Acetic Anhydride

n-Octyl-acrylate (175 mg, 0.95 mmol, 3 eq) was added to a stirred solution of G1-PPI—(NH₂)₄ dendrimer (100 mg, 0.32 mmol) in IPA (1 mL). The reaction was heated for 16 h at 60° C., and was then concentrated in vacuo and dried. The residue was redissolved in DCM (1 mL) and triethylamine (185 μL, 134 mg, 1.33 mmol, 4.2 eq) and acetic anhydride (125 μL, 135 mg, 1.33 mmol, 4.2 eq) were added. The reaction mixture was stirred for 2 h at room temperature and was then diluted with chloroform. The chloroform layer was washed with a saturated NaHCO₃-solution (1×25 mL) and with brine (1×25 mL), was dried using Na₂SO₄ and concentrated in vacuo to yield the product (365 mg, 0.35 mmol. Terminating unit groups are a mixture of n-octyl-acrylate and acetyl groups.

¹H NMR (400 MHz, Chloroform-d) δ 6.63 (m, 1H), 4.07 (dtd, J=13.4, 6.8, 2.7 Hz, 6H), 3.58 (dt, J=14.3, 5.7 Hz, 6H), 3.30 (p, J=7.4 Hz, 7H), 2.76 (t, J=7.3 Hz, 1H), 2.67-2.52 (m, 6H), 2.52-2.29 (m, 12H), 2.17-2.03 (m, 9H), 1.96 (d, J=4.8 Hz, 3H), 1.63 (p, J=6.9 Hz, 14H), 1.30 (dd, J=14.1, 7.6 Hz, 34H), 0.88 (t, J=6.7 Hz, 9H). MALDI-TOF-MS (CHCA matrix, positive reflector mode): 3× n-octyl-acrylate Obs. m/z=(M+H)⁺ 1037.80, (M+Na)⁺ 1059.77. Calculated: C₅₇H₁₀₈N₆O₁₀ (exact mass 1036.81; molecular weight 1037.52). 4× octyl-acrylate Obs. m/z=(M+H)⁺ 1221.95, (M+Na)⁺ 1243.93. Calculated: C₆₈H₁₂₈N₆O₁₂ (exact mass 1220.96; molecular weight 1221.80).

### Example K2. G2 PPI Dendrimer Modified with n-Octyl-Acrylate and Acetic Anhydride

Similar to Example K1 G2-PPI—(NH₂)₈ (102 mg, 0.13 mmol) was reacted in step 1 with n-octyl-acrylate (169 mg, 0.92 mmol, 6.95 eq) in IPA (1 mL), and in step 2 with acetic anhydride (125 μL, 136 mg, 1.33 mmol, 10 eq) using DIPEA (231 μL, 172 mg, 1.33 mmol, 10 eq) in DCM (1 mL). Terminating unit groups are a mixture of n-octyl-acrylate and acetyl groups.

¹H NMR (400 MHz, Chloroform-d) δ 4.06 (dt, J=13.5, 6.8 Hz, 14H), 3.58 (dt, J=13.5, 7.5 Hz, 14H), 3.31 (q, J=9.4, 8.6 Hz, 15H), 2.76 (t, J=7.3 Hz, 1H), 2.70-2.52 (m, 14H), 2.40 (m, 36H), 2.10 (d, J=11.6 Hz, 21H), 1.96 (s, 3H), 1.85-1.44 (m, 38H), 1.45-1.10 (m, 74H), 0.88 (t, J=6.7 Hz, 21H). MALDI-TOF-MS (CHCA matrix, positive reflector mode): 6× n-octyl-acrylate Obs. m/z=(M+H)⁺ 2215.75, (M+Na)⁺ 2237.73. Calculated: C₁₂₂H₂₃₂N₁₄O₂₀ (exact mass 2213.76; molecular weight 2215.28). 7× octyl-acrylate Obs. m/z=(M+H)⁺ 2399.90, (M+Na)⁺ 2421.88. Calculated: C₁₃₃H₂₅₂N₁₄O₂₂ (exact mass 2397.90; molecular weight 2399.56).

### L. Functionalization of Primary Amines with Acryl-Amides Followed by Epoxidation of the Remaining Secondary Amines (Here, the Products are Mixtures of Polyvalent Molecules)

### Example L1. G1 PPI Dendrimer Modified with n-Octyl-Acryl-Amide and 1,2-Epoxydodecane

n-Octyl-acrylamide (174 mg, 0.95 mmol, 3 eq) was added to a stirring solution of G1-PPI—(NH₂)₄ (100 mg, 0.32 mmol) in IPA (1 mL). The reaction mixture was stirred at 60° C. for 160h to complete the first step. Then 1,2-epoxydodecane (582 mg, 3.16 mmol, 10 eq) and IPA (0.5 mL) were added and the reaction mixture was stirred for another 16 h at 60° C. The mixture was cooled down and concentrated in vacuo. The residue was stirred in MeCN and cooled down to −20° C. allowing the product to phase separate from the MeCN solvent. The supernatant was carefully removed using a pipette and the trituration procedure was repeated twice giving an amber oil (495 mg, 0.28 mmol, 76%). Terminating unit groups are a mixture of n-octyl-acrylamide and epoxide-derived groups.

¹H NMR (400 MHz, Chloroform-d) δ 6.79 (s, 3H), 3.76 (m, 5H), 3.62 (m, 5H), 3.20 (q, J=7.7, 6H), 3.01-2.77 (m, 3H), 2.77-2.12 (m, 37H), 1.68-1.06 (m, 138H), 0.88 (t, J=6.8 Hz, 24H). MALDI-TOF-MS (DCTB matrix, positive reflector mode): Obs. m/z=(M+H)⁺ 1788.71, Calculated: C₁₀₉H₂₂₃N₉O₈ (exact mass 1786.73; molecular weight 1788.04).

### Example L2. G2 PPI Dendrimer Modified with n-Octyl-Acryl-Amide and 1,2-Epoxydodecane

Similar to Example L1 G2-PPI—(NH₂)₈ (100 mg, 0.13 mmol) was reacted in step 1 with n-octyl-acryl-amide (142 mg, 0.78 mmol, 6 eq) in IPA (1 mL), and in step 2 with 1,2-epoxydodecane (477 mg, 2.59 mmol, 20 eq). Yield: an amber viscous oil (463 mg, 96%). Terminating unit groups are a mixture of n-octyl-acrylamide and epoxide-derived groups.

¹H NMR (400 MHz, Chloroform-d) δ 6.96 (s, 6H), 3.84 (d, J=8.1 Hz, 10H), 3.61 (s, 10H), 3.18 (t, J=7.0 Hz, 12H), 2.99-2.77 (m, 6H), 2.77-2.09 (m, 90H), 1.27 (d, J=5.6 Hz, 280H), 0.88 (t, J=6.7 Hz, 48H). MALDI-TOF-MS (DCTB matrix, positive reflector mode): Obs. m/z=(M+H)⁺ 3717.55, Calculated: C₂₂₆H₄₆₂N₂₀O₁₆ (exact mass 3713.60; molecular weight 3716.31).

### Example 2—Solubility of the Polyvalent Molecules

The solubility of various of the polyvalent molecules was tested in ethanol, in iso-propanol and in tri-caprylin, at a concentration of 20 mg material per gram of solvent. Spontaneous dissolution upon stirring at room temperature giving a clear solution is a mark for solubility. It was found that the polyvalent molecules of Examples A1, A2, A4, A6, A7, A8, A9, A10, A11, B1, B2, B3, B4, C2, E1, F1, G1, G2, G3, G5, G7, G8, H1, J10 and J11 were soluble in all three solvents at room temperature. Example C1 was soluble in isopropanol and tricaprylin. Example D4 was soluble in the three solvents upon heating to 37 degrees. Please note that not all prepared materials were tested.

These data show that the polyvalent molecules are well soluble for processing, for example in ethanol or iso-propanol. The data also show solubility in tri-caprylin, implying that the molecules presumably have affinity for the hydrophobic interior of the nanoparticles of the invention.

### Example 3—Constructing Polyvalent Molecule Comprising Nanoparticles

Nanoparticle formulations self-assemble based on ionic and hydrophobic interactions. The components are prepared at the desired concentrations in their respective organic solvent (lipids and other structural components) or aqueous buffer (nucleic acid payloads). The solutions are then brought together via rapid mixing techniques encompassing microfluidic or T-junction mixing. An excess of aqueous buffer is essential for the formation process. When used herein, an excess of aqueous buffer refers to a ratio of (aqueous buffer):(organic solvent) (based on volume) of at least 2:1 or higher, e.g. 2.2:1, 2.5:1, 2.8:1 or 3:1 or higher. After initial mixing the small fraction of organic solvent is removed, for example with dialysis or centrifugal filtration.

These steps yield lipid nanoparticles to which apolipoprotein (the stabiliser) is added via a rapid mixing technique such as microfluidic or T-junction mixing or using for example a drip method. After apolipoprotein addition, residual protein needs to be removed by dialysis or centrifugal filtration. Finally, the sample is concentrated to a desired concentration.

The nucleic acid nanoparticle thus produced comprise:
(a) a polyvalent molecule;
(b) an apolipoprotein stabiliser;
(c) a nucleic acid;
(d) a sterol;
(e) a phospholipid; and
(f) optionally a lipid.

Accordingly, different compositions as described in Table S3, Table S7 and Table S8, using the indicated polyvalent molecules were prepared, combined with the following control preparations:

LNP controls are “standard LNPs” as used for the clinically approved LNP-siRNA formulations as described in Akinc A, et al. The Onpattro story and the clinical translation of nanomedicines containing nucleic acid-based drugs. Nat Nanotechnol. 2019 December; 14(12):1084-1087. doi: 10.1038/s41565-019-0591-y). The composition is (mol %), thereby disregarding the presence of nucleic acid:
DLin-MC3-DMA 50%
DSPC 10%
Cholesterol 38.5%
PEG-DMG 1.5%

MC3-based aNP controls are apolipoprotein nanoparticles where instead of a polyvalent molecule as described herein DLin-MC3-DMA is used to complex nucleic acid. The compositions are listed in Table S3 and Table S8.

TABLE S3
Employed component amounts in the formulation of siRNA-containing
nanoparticles. The reference nanoparticles are LNPs and aNPs
(apolipoprotein nanoparticles) containing the ionizable cationic
lipid DLin-MC3-DMA. The aNPs of the invention have polyvalent
ionizable material to complex siRNA.

| | | | | aNP (exemplary | |
|---|---|---|---|---|---|
| | | | | aNP according to | |
| | LNP (MC3) | aNP (MC3) | | the invention) | |
| N/P ratio | 6 | 6 | | 6 | |
| Type of ionizable- | DLin- | DLin- | | Polyvalent | |
| cationic lipid | MC3-DMA | MC3-DMA | | material | |
| | (or MC3) | (or MC3) | | | |
| Ionizable-cationic | 2.42 mg | 2.42 mg | | 0.91-1.67 mg | |
| lipid | | | | (depending on the | |
| | | | | specific material) | |
| DSPC | 0.60 mg | — | | — | |
| DMPC | — | 2.50 | mg | 2.50 | mg |
| DMG-PEG2000 | 0.28 mg | — | | — | |
| Cholesterol | 1.12 mg | 1.35 | mg | 1.35 | mg |
| Tricaprylin (TG) | — | 10 | mg | 10 | mg |
| Apo A1 | — | 1.0 | mg | 1.0 | mg |
| siRNA | 0.20 mg | 0.20 | mg | 0.20 | mg |

The aNPs of the invention were prepared using the amounts as given in the Table above, in combination with 1.00 (for A1), 1.13 (A2), 1.60 (A3), 1.27 (A6), 1.20 (A7), 1.18 (A8), 1.11 (A11), 1.16 (A14), 0.98 (B2), 1.67 (D4), 1.20 (F1), 1.16 (G2), 1.48 (G6), 1.15 (G7), 1.11 (G8), 0.97 (G9) or 0.91 (H1) mg of the polyvalent material. DMG-PEG 2000 is a synthetic lipid and is a PEGylated myristoyl diglyceride. Tri-caprylin is glyceryl tri-n-octanoate and is a tri-glyceride (TG).

TABLE S7
DLS data of siRNA-aNPs of the invention containing polyvalent
ionizable molecule A2. The employed formulation composition
of the aNPs is as according to Table S3, apart from the single
mutations given in the first two columns. In all cases nanoparticles
with diameters smaller than 200 nm are formed.

| | | DLS | |
|---|---|---|---|
| | | Hydrodynamic | DLS |
| Variation in aNP composition | | diameter (nm) | PDI (—) |
| N/P = 3 | — | 173 | 0.79 |
| N/P = 9 | — | 62 | 0.24 |
| N/P = 6 | No Triglyceride | 122 | 0.16 |
| | Low Triglyceride (5 mg) | 60 | 0.08 |
| | Low Cholesterol (0.54 mg) | 95 | 0.07 |
| | High Cholesterol (3.37 mg) | 103 | 0.24 |
| | DMPC | 91 | 0.18 |
| | DSPC | 60 | 0.07 |
| | DPPC | 61 | 0.08 |
| | POPC | 123 | 0.08 |

TABLE S8
Employed component amounts in the formulation of mRNA-
loaded nanoparticles. The reference nanoparticles are
LNPs and aNPs (apolipoprotein nanoparticles), containing
the ionizable cationic lipid ALC-0315. The aNPs of the
invention contain polyvalent ionizable material A2.
In vitro experiments

| | | | aNP (exemplary |
|---|---|---|---|
| | LNP | aNP | aNP according to |
| | (ALC-0315) | (ALC-0315) | the invention) |
| N/P ratio | 6 | 6 | 6 |
| Type of ionizable- | ALC-0315 | ALC-0315 | Polyvalent |
| cationic lipid | | | material A2 |
| Ionizable-cationic | 1.43 mg | 1.43 mg | 0.55 mg |
| lipid | | | |
| DSPC | 0.30 mg | — | — |
| DMPC | — | 1.19 mg | 1.19 mg |
| PEG-DMG | 0.14 mg | — | — |
| Cholesterol | 0.56 mg | 0.65 mg | 0.65 mg |
| Tricaprylin (TG) | — | 2.39 mg | 2.39 mg |
| Apo A1 | — | 0.48 mg | 0.48 mg |
| mRNA (RIBOPRO | 0.10 mg | 0.10 mg | 0.10 mg |
| Fluc mRNA) | | | |

In vivo experiments

| | aNP (exemplary |
|---|---|
| | aNP according to |
| | the invention) |
| N/P ratio | 6 |
| Type of ionizable- | Polyvalent |
| cationic lipid | material A2 |
| Ionizable-cationic | 0.65 mg |
| lipid | |
| DSPC | — |
| DMPC | 1.40 mg |
| PEG-DMG | — |
| Cholesterol | 0.76 mg |
| Tricaprylin (TG) | 2.80 mg |
| Apo A1 | 0.58 mg |
| mRNA (TriLink | 0.10 mg |
| CleanCap ® Fluc | |
| mRNA (5 moU)) | |

### Example 4—Preparation of the Nanoparticles

Materials. 1,2-Dimyristoyl-sn-glycero-3-phosphocholine (DMPC), 1,2-dimyristoyl-rac-glycero-3-methoxypolyethylene glycol-2000 (DMG-PEG), 1,2-distearoyl-sn-glycero-3-phosphocholine (DSPC) were purchased from Avanti Polar Lipids New Jersey, US. Cholesterol and tri-caprylin were obtained from Sigma Aldrich. D-Lin-MC3-DMA (MC3) was obtained from the University of British Columbia, Centre for Organic Chemistry, Vancouver, Canada. ALC-0315 was synthesized in house. Recombinant apo A1 was expressed and purified from ClearColi BL21(DE3) cells in house. Firefly luciferase and negative control Dicer-substrate siRNAs were obtained from Integrated DNA Technologies (IDT), Iowa, US. 10.

Preparation of siRNA-Loaded Nanoparticles

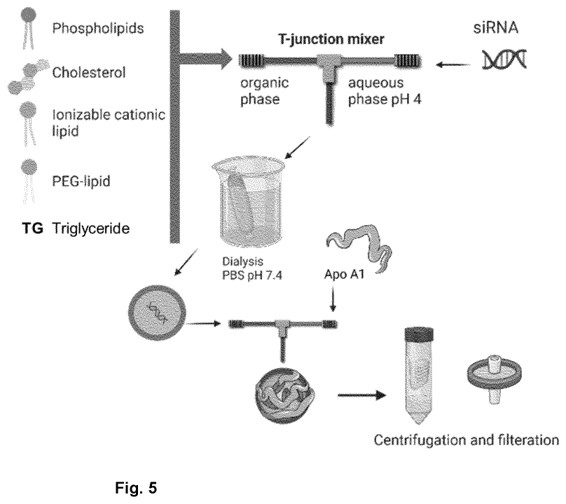

A scheme of the formulation process is found in FIG. 5. The specific formulation compositions of the aNPs of the invention, the aNP control particles and the LNP control particles can be found in Table S3. Variations in composition were also evaluated, see Table S7.

siRNA-loaded aNPs were formulated by rapid mixing using a T-junction device. The lipid molecules (phospholipid, cholesterol, triglyceride and ionizable material for the aNPs; phospholipid, cholesterol, ionizable material and PEG-lipid for the LNPs) were dissolved in ethanol and rapidly mixed with sodium acetate buffer (25 mM, pH 4) containing 200 μg anti-firefly luciferase siRNA or scrambled siRNA. Organic and aqueous phases were mixed and directly collected in a 12-14 kDa MWCO dialysis membrane (Spectra/Por™). Nanoparticle formulations were dialyzed against 1×PBS (137 mM NaCl, 2.7 mM KCl, 10 mM Na2HPO4 and 1.8 mM KH2PO4, pH 7.4) overnight at 4° C. and stirred at 150 rpm. PBS was refreshed after approximately 4 hours. The subsequent day, the formulation sample was collected from the dialysis bag and the volume was determined. In case control LNP particles were formulated, the apolipoprotein addition step was skipped. For the aNP particles, apolipoprotein A1 was dissolved in PBS and was added to the formulations by rapid T-junction-based mixing.

Resulting samples were incubated at room temperature for one hour. The siRNA-aNPs were filtered through an 0.2 μm filter and concentrated by centrifugal filtration in a 100,000 MWCO filter at 1100 G. Samples were concentrated to 1.5 mL and stored at 4° C. until further use. For use on cells, the samples were kept sterile after the 0.2 μm filtration step.

Preparation of mRNA-Loaded Nanoparticles

A scheme of the formulation process is found in FIG. 5. The specific formulation compositions of the aNPs of the invention, the aNP control particles and the LNP control particles can be found in Table S8.

mRNA-loaded aNPs were formulated by rapid T-junction mixing in a RNase free culture hood. The lipid molecules (phospholipid, cholesterol, triglyceride and ionizable material for the aNPs; phospholipid, cholesterol, ionizable material and PEG-lipid for the LNPs) were dissolved in ethanol and rapidly mixed with sodium acetate buffer (25 mM, pH 4) containing 100 μg mCherry mRNA (TriLink, CleanCap, 5 moU). Organic and aqueous phases were mixed and directly collected in a 12-14 kDa MWCO dialysis membrane (Spectra/Por™). Nanoparticle formulations were dialyzed against 1×PBS (137 mM NaCl, 2.7 mM KCl, 10 mM Na2HPO4 and 1.8 mM KH2PO4, pH 7.4) overnight at 4° C. and stirred at 150 rpm. PBS was refreshed after approximately 4 hours. The subsequent day, the formulation sample was collected from the dialysis bag and the volume was determined. In case control LNP particles were formulated, the apolipoprotein addition step was skipped. For the aNP particles, apolipoprotein A1 was dissolved in PBS and was added to the formulations by rapid T-junction mixing.

Resulting samples were incubated at room temperature for 15 minutes. The mRNA-aNPs were filtered through an 0.2 μm filter and concentrated by centrifugal filtration in a 100,000 MWCO filter at 1100 G. Samples were concentrated to 0.5 mL and stored at 4° C. until further use. For use on cells, the samples were kept sterile after the 0.2 μm filtration step.

### Examples 5—Characterizations of the Nanoparticles

Determination of the siRNA Encapsulation Efficiency

The Quant-iT RiboGreen assay (Thermo Fisher) was used to quantify the amount of siRNA loaded inside the formulated particles. The assay was performed using a black 96-well plate. The formulation sample with a theoretical siRNA concentration of 133.3 μg/mL, was diluted 200 times in TE buffer (10 mM Tris-HCl, 1 mM, EDTA, pH 7.5 in DEPC-treated water) and with a TE buffer containing 2% Triton™ X-100, in a black 96-well plate to a total volume of 100 μl. The Triton detergent will disrupt the lipid-based nanoparticles; therefore, all siRNA (retained plus unretained) becomes accessible for the Quant-iT RiboGreen reagent. Controls of siRNA of known concentration (28.6 μg/mL) were diluted 53.3 times in both TE buffer and TE buffer containing 2% Triton in a 96-well plate to a total volume of 100 μl. Next, RiboGreen reagent was diluted 200 times with TE buffer and with TE buffer containing Triton. 100 μl of this dilution was added to each well containing sample or control to bring the total volume in the plate to 200 μl. Subsequently, the samples' fluorescence was measured on a Tecan Spark® microplate reader at an excitation wavelength of 480 nm and emission wavelength of 520 nm.

The recovery of siRNA was determined as:

$$\frac{Amount of siRNA ( retained + u n r e t a i n e d )}{Total amount of siRNA ( used in the formulation )} \times 100 \% \\ ( 1 - ( \frac{Amount of unretained siRNA}{Amount of siRNA ( retained + u n r e t a i n e d )} ) ) \times 100 \%$$

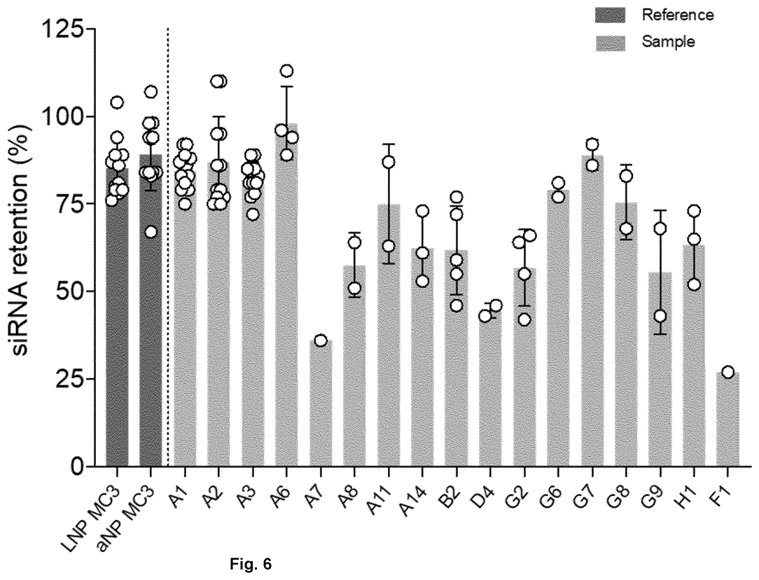

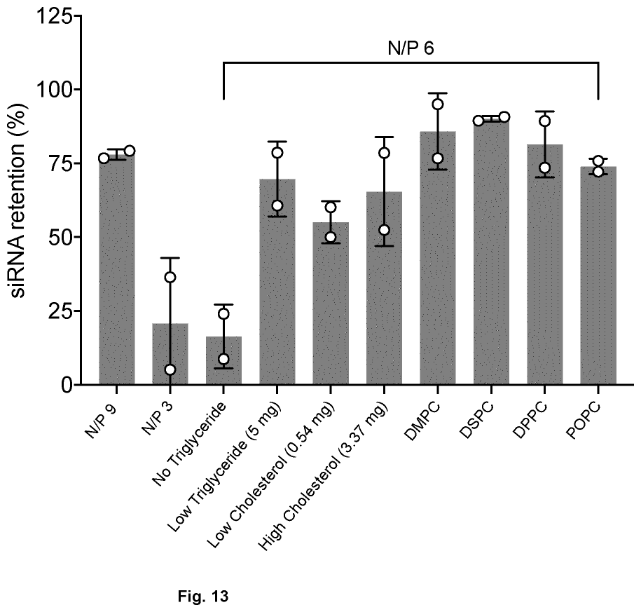

The siRNA retention=recovery×entrapment. Results are reported in FIG. 6 and FIG. 13.

Determination of the mRNA Encapsulation Efficiency

This was done in a similar way as described for determining the siRNA encapsulation efficiency. Results are reported in Table S9.

TABLE S9
Recovery, entrapment, and retention percentages
of mRNA in aNPs as prepared from the shown polyvalent
material and determined by Ribogreen assay.

| | | Polyvalent | Recovery | Entrapment | Retention |
|---|---|---|---|---|---|
| Entry | Experiment | Material | (%) | (%) | (%) |
| 1 | Vitro | A2 | 27% | 87% | 23% |
| 2 | Vivo | A2 | 57% | 88% | 50% |

### Determination of Phospholipid and Cholesterol Recovery

The recovery of phospholipid and cholesterol in the prepared nanoparticles is quantified by performing Phospholipid FS and Cholesterol FS assays (DiaSYS), which are enzymatic colorimetric tests. Buffer containing color reagent (190 μl) is added to 10 μl of the nanoparticle sample in a transparent 96-well plate and incubated at 37° C. for 30 minutes. The absorbance is measured at 600 nm using a Tecan Spark® plate reader. Results are reported in Table S4.

TABLE S4
Cholesterol, phospholipid, and apo-A1 recoveries in siRNA-
aNPs. The number n represents the number of individual repeats
of each formulation (A1 n = 8 means that A1 was formulated
8 different times, and the average of the recovery values
was determined). All particles show initial stability.

| | Polyvalent | Cholesterol | Phospholipid | Apo A1 |
|---|---|---|---|---|
| Entry | material | (recovery %) | (recovery %) | (recovery %) |
| 1 | A1 | 81% ± 12 | 89% ± 13 | 44% ± 17 |
| | | (n = 8) | (n = 10) | (n = 8) |
| 2 | A2 | 87% ± 8 | 96% ± 13 | 61% ± 16 |
| | | (n = 8) | (n = 8) | (n = 8) |
| 3 | A3 | 87% ± 6 | 77% ± 2 | 35% ± 13 |
| | | (n = 2) | (n = 2) | (n = 2) |
| 4 | A6 | — | 93% ± 4 | — |
| | | | (n = 2) | |
| 5 | A8 | — | 48% ± 6 | 34% |
| | | | (n = 2) | (n = 1) |
| 6 | A11 | — | 50% ± 11 | 27% |
| | | | (n = 2) | (n = 1) |
| 7 | A14 * | — | 66% ± 24 | — |
| | | | (n = 2) | |
| 8 | B2 * | — | 116% ± 40 | — |
| | | | (n = 2) | |
| 9 | D4 | 83% ± 6 | 78% ± 1 | 18% ± 8 |
| | | (n = 2) | (n = 2) | (n = 2) |
| 10 | G2 | 99% ± 1 | 90% ± 16 | 37% ± 8 |
| | | (n = 2) | (n = 3) | (n = 3) |
| 11 | G6 | — | 63% ± 3 | 26% |
| | | | (n = 2) | (n = 1) |
| 12 | G7 | — | 75% ± 4 | 31% |
| | | | (n = 2) | (n = 1) |
| 13 | G8 | | 63% ± 5 | 26% |
| | | | (n = 2) | (n = 1) |
| 14 | G9 | — | 65% ± 6 | 33% |
| | | | (n = 2) | (n = 1) |
| 15 | H1 * | — | 84% | — |
| | | | (n = 2) | |

* Indicates phase separation after two days of storage in the refrigerator at 4° C.

### Determination of Apolipoprotein a 1 (Apo a 1) Recovery

The amount of apolipoprotein A1 in the nanoparticle formulation was determined using the apolipoprotein A1 FS assay (DiaSYS)®. It is an immunoturbidimetric test based on the interaction between the anti-apo A1 antibody and apo A1 present in the sample. TRIS buffer (200 μl) was added to 10 μl of the nanoparticle sample in a transparent 96-well plate. After incubating the plate at 37° C. for 5 minutes, the absorbance was measured using a Tecan Spark® microplate reader at a wavelength 580 nm. TRIS (40 μl) containing apolipoprotein A1 antibody was added to the same 96-well plate, and the solution was incubated at 37° C. for 5 minutes before measuring the absorbance at the same setting. Results are reported in Table S4.

### Dynamic Light Scattering (DLS)

The hydrodynamic diameter of the formulated particles was determined by a number-weighted mean diameter provided by dynamic light scattering (DLS) using a Zetasizer Nano ZS in combination with a Malvern Zetasizer NanoSampler (Malvern Instruments, Worcestshire, UK). The size dispersity was measured as the poly dispersity index (PDI). For the DLS measurements, 100 μl of the formulated nanoparticle was diluted into 700 μl of PBS (137 mM NaCl, 2.7 mM KCl, 10 mM Na2HPO4 and 1.8 mM KH2PO4) and equilibrated at room temperature before the analysis. Each sample was measured 5 times, 10 runs of each 10 seconds, at wavelength 633 nm, without fixing the attenuator and measurement position. Results are reported in Table S5, Table S7 and Table S10.

TABLE S5
DLS (dynamic light scattering) analysis of siRNA-aNPs. Hydrodynamic
diameters (number mean) and PDIs (polydispersity index) of
the apolipoprotein nanoparticles (aNPs) are given.

| | | DLS | | DLS | |
|---|---|---|---|---|---|
| | Polyvalent | Hydrodynamic diameter | | PDI | |
| Entry | material | (nm) | | (—) | |
| 1 | A1 | 93 ± 15 | (n = 10) | 0.10 ± 0.03 | (n = 10) |
| 2 | A2 | 57 ± 6 | (n = 10) | 0.16 ± 0.03 | (n = 10) |
| 3 | A3 | 55 ± 0.7 | (n = 2) | 0.14 ± 0.01 | (n = 2) |
| 4 | A6 | 48 ± 1 | (n = 3) | 0.14 ± 0.02 | (n = 3) |
| 5 | A7 ** | 115 | (n = 1) | 0.18 | (n = 1) |
| 6 | A8 | 84 | (n = 1) | 0.15 | (n = 1) |
| 7 | A11 | 136 ± 45 | (n = 2) | 0.14 ± 0.04 | (n = 2) |
| 8 | A14 * | 100 | (n = 3) | 0.10 | (n = 3) |
| 9 | B2 * | 67 | (n = 1) | 0.09 | (n = 1) |
| 10 | D4 | 56 | (n = 2) | 0.16 | (n = 2) |
| 11 | F1 ** | 32 | (n = 1) | 1 | (n = 1) |
| 12 | G2 | 55 ± 4 | (n = 2) | 0.10 ± 0.01 | (n = 2) |
| 13 | G6 | 83 ± 4 | (n = 2) | 0.13 ± 0.08 | (n = 2) |
| 14 | G7 | 54 ± 1 | (n = 2) | 0.2 ± 0.06 | (n = 2) |
| 15 | G8 | 84 ± 45 | (n = 2) | 0.2 ± 0.05 | (n = 2) |
| 16 | G9 | 73 ± 10 | (n = 2) | 0.18 ± 0.08 | (n = 2) |
| 17 | H1 * | 66 ± 7 | (n = 3) | 0.45 ± 0.43 | (n = 3) |

* Indicates phase separation after two days storage in the fridge at 4° C.,
** Indicates phase separation after overnight dialysis.
The standard deviation was measured considering the number of replicates of the same formulation (individual repeats).

TABLE S10
DLS (dynamic light scattering) of mRNA-aNPs. Hydrodynamic
diameters (number mean) and PDIs (polydispersity
index) of the nanoparticles are given.

| | | | DLS | |
|---|---|---|---|---|
| | | Polyvalent | Hydrodynamic | DLS |
| Entry | Experiment | Material | diameter (nm) | PDI (—) |
| 1 | Vitro | A1 | 88 | 0.18 |
| 2 | Vitro | A2 | 53 | 0.27 |
| 3 | Vivo | A2 | 63 | 0.08 |

### Cryogenic Transmission Electron Microscopy (Cryo-TEM)

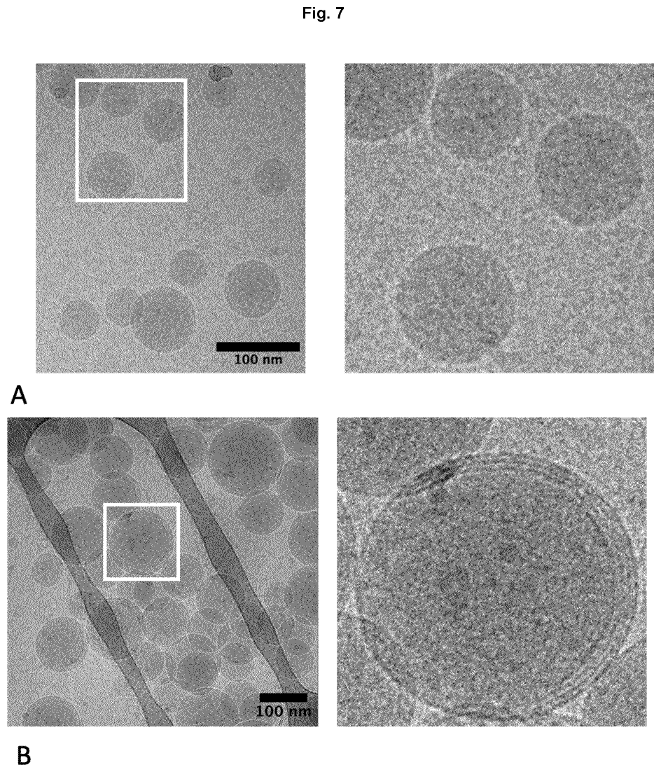

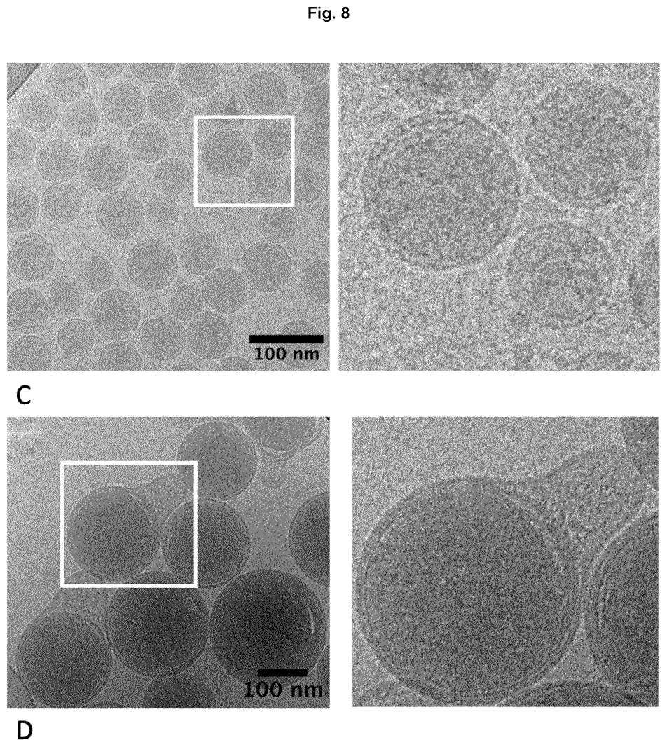

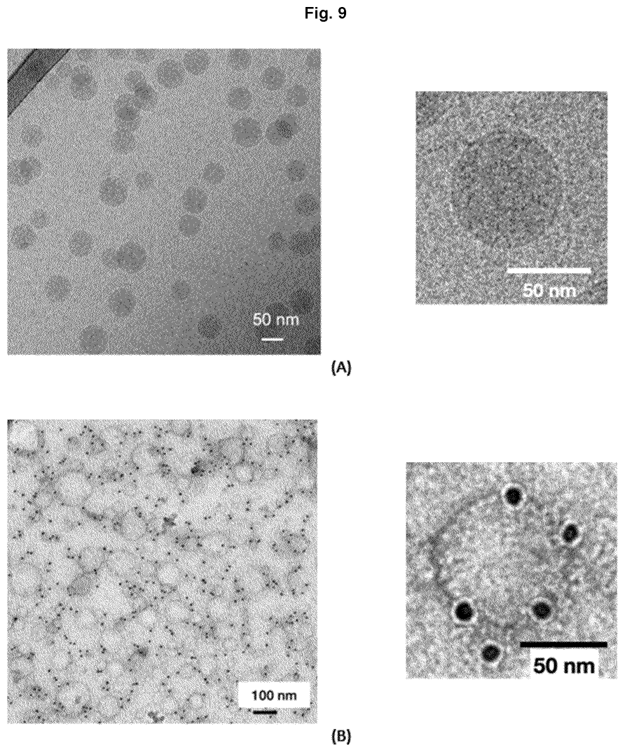

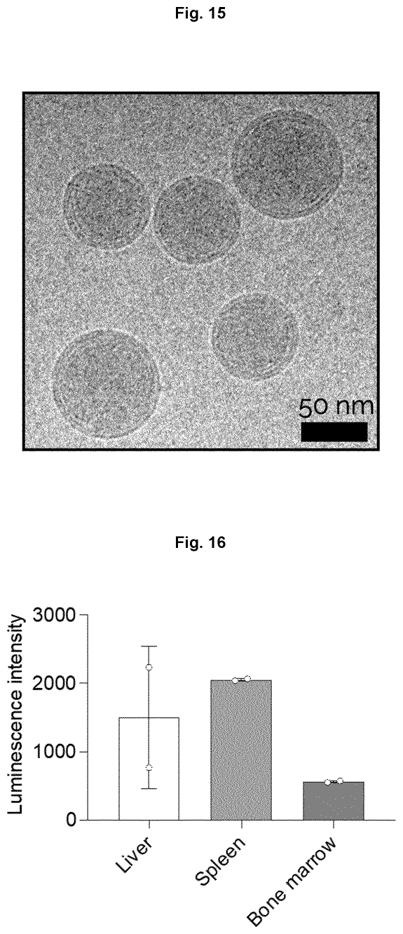

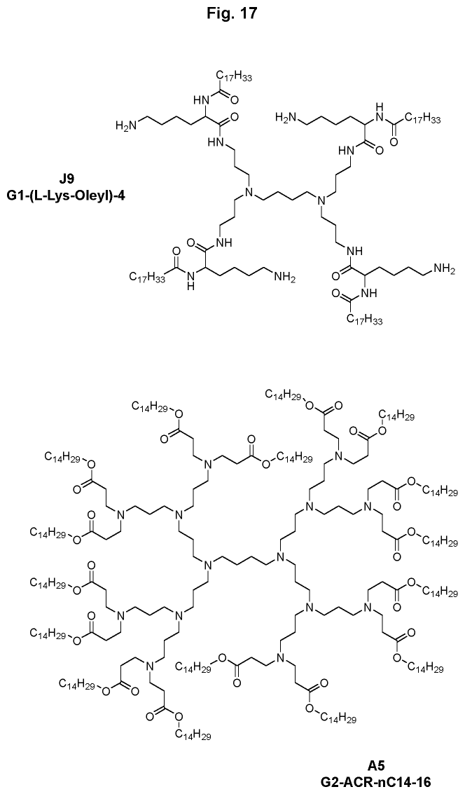

Size and shape of the nanoparticles was also assessed by cryo-transmission electron microscopy (cryo-TEM). The surface of 200-mesh lacey carbon supported copper grids (Electron Microscopy Sciences) was plasma charged for 40 seconds using a carbon coater (Cressington 208). Subsequently, 3 μl of nanoparticle sample (˜1 mg protein/ml) was pipetted on a grid and vitrified into a thin film by plunge vitrification in liquid ethane. This step was performed by using an automated robot (FEI Vitrobot Mark IV). Cryo-TEM imaging was acquired on the cryo-transmission electron microscope TITAN (Thermo Fisher), equipped with a field emission gun (FEG), a post-column Gatan imaging filter (model 2002) and a post-GIF 2 k×2 k Gatan CCD camera (model 794). The imaging was performed at 300 kV acceleration voltage in bright-field TEM mode with zero-loss energy filtering at 24,000× magnification (dose rate of 11.8 e−/Å2·s), and 1s acquisition time. Pictures and results are reported in Table S6, FIG. 7, FIG. 8, FIG. 9 (A), FIG. 15.

TABLE S6
Nanoparticle diameters of siRNA-
aNPs as recorded with cryo-TEM.

| | | Cryo-TEM | |
|---|---|---|---|
| | Polyvalent | Diameter | Cryo-TEM |
| Entry | material | (nm) | PDI (—) |
| 1 | A1 | 98 ± 24 | 0.06 |
| | | (n = 20) | (n = 20) |
| 2 | A2 | 55 ± 11 | 0.04 |
| | | (n = 20) | (n = 20) |
| 3 | A6 | 53 ± 11 | 0.02 |
| | | (n = 20) | (n = 20) |
| 4 | A14 * | 174 ± 33 | 0.04 |
| | | (n = 8) | (n = 8) |

* Indicates phase separation after two days storage in the fridge at 4° C. The number of individual particles were considered for measuring the standard deviation.

Scanning Electron Microscopy in Combination with Immuno-Gold Staining

Pictures and results are reported in FIG. 9 (B).

Cytotoxicity Measurements on Apolipoprotein Nanoparticles (aNPs)

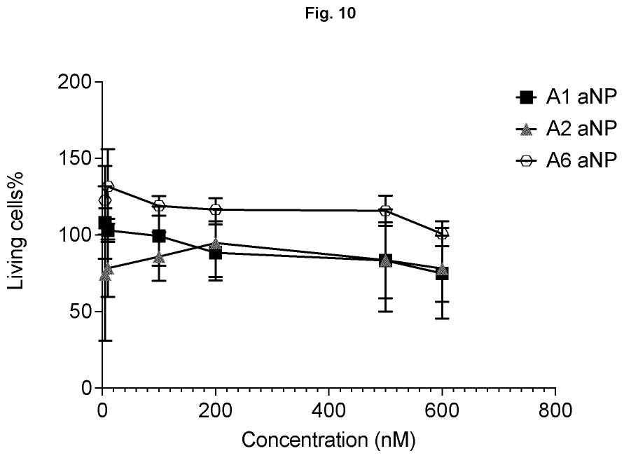

MTS assays were carried out on multiple apolipoprotein nanoparticles (aNPs) of the invention. These assays were executed in line with protocols that are known in the art. Results are reported in FIG. 10.

### In Vitro Silencing (Dose-Response Curve)

The in vitro silencing experiments were performed in a RAW264.7 cell line transfected with the pmirGLO plasmid (containing Renilla luciferase and Firefly luciferase expressing gene sequences). The cells were cultured until 80% confluency in a T75 cell culture flask. The cells were detached, counted, and seeded at 10000 cells/well in a 96-well plate. After overnight resting, the cells were transfected with nanoparticles containing anti Firefly luciferase siRNA at a range of concentrations. After 48-hour incubation, the old medium was washed off with 1×PBS. Lysate phosphate buffer (Dual-Luciferase® Reporter Assay System, Promega) was added to lyse the cells. 10 μl of the cell lysate was transferred to a white 96-well flat-bottom plate. Subsequently, 40 μl of ONE-Glo™ reagent (Dual-Luciferase® Reporter Assay System, Promega) was added and luminescence was measured with a Tecan Spark® microplate reader at an integration time of 500 ms, and settle time of 1000 ms. Since the luminescence peak should be in the range of 550-570 nm, a luminescence scan was performed to confirm the wavelength. Next, 40 μl of Stop and Glo reagent (Dual-Luciferase® Reporter Assay System, Promega) was added to each well and the luminescence was measured again in the same way. The relative remaining Firefly luciferase luminescence was calculated. First, the Firefly luminescence was normalized with the Renilla luminescence. Subsequently, the normalized sample signal was expressed as a percentage of the untreated sample signal. This yields the following formula:

$$\frac{( \frac{Firefly luminescence}{Renilla luminescence} ) Sample}{( \frac{Firefly luminescence}{Renilla luminescence} ) Untreated} \times 100 \%$$

Results are reported in FIG. 11.

In Vitro Silencing (Scrambled siRNA Versus Anti Firefly siRNA)

The in vitro silencing experiments were performed in a RAW264.7 cell line transfected with the pmirGLO plasmid (containing Renilla luciferase and Firefly luciferase expressing gene sequences). The cells were cultured until 80% confluency in a T75 cell culture flask. The cells were detached, counted, and seeded at 10000 cells/well in a 96-well plate. After overnight resting, the cells were transfected with nanoparticles containing either scrambled non-specific siRNA or anti Firefly luciferase siRNA at 100 nM. After 48-hour incubation, the old medium was washed off with 1×PBS. Lysate phosphate buffer (Dual-Luciferase® Reporter Assay System, Promega) was added to lyse the cells. 10 μl of the cell lysate was transferred to a white 96-well flat-bottom plate. Subsequently, 40 μl of ONE-Glo™ reagent (Dual-Luciferase® Reporter Assay System, Promega) was added and luminescence was measured with a Tecan Spark® microplate reader at an integration time of 500 ms, and settle time of 1000 ms. Since the luminescence peak should be in the range of 550-570 nm a luminescence scan was performed. Then, 40 μl of Stop and Glo reagent (Dual-Luciferase® Reporter Assay System, Promega) was added to each well and the luminescence was measured again in the same way. The relative remaining Firefly luciferase luminescence was calculated. First, for all samples the Firefly luminescence was normalized with the Renilla luminescence. Subsequently, the normalized sample signal was expressed as a percentage of the untreated sample signal. This yields the following formula:

$$\frac{( \frac{Firefly luminescence}{Renilla luminescence} ) Sample}{( \frac{Firefly luminescence}{Renilla luminescence} ) Untreated} \times 100 \%$$

Results are reported in FIG. 12.

### In Vitro Firefly Luciferase Expression

For the detection of Firefly luciferase expression in vitro, the ONE-Glo™ Luciferase Assay System (Promega) was used. RAW264.7 cells were seeded at a density of 35 k cells per well in a 96-well plate in 100 μL. After approximately 6 hours the RAW264.7 cells were transfected with 10 μL Firefly luciferase mRNA (RIBOPRO) using aNPs or LNPs at a dose of 100 ng mRNA per well. 24 hours after transfection, cell viability was determined by a CellTiter 96® AQueous One Solution Cell Proliferation assay (MTS) (Promega) assay. 10 μL reagent was added to the cells, and absorbance was measured at 495 nm after an hour of incubation (37° C., 5% CO2). Subsequently, the cells were washed three times with 1×PBS, and then 40 μL ONE-Glo™ Luciferase Assay Reagent was added to each well. After complete lysis of the cells, the cell lysates were transferred to a white flat bottom 96-well plate. Luminescence was measured using a Tecan Spark plate reader. Luciferase expression was expressed as luciferase intensity after normalization to the cell viability and subtracting background signal. Results are reported in FIG. 14.

### In Vivo Firefly Luciferase Expression

For the detection of Firefly luciferase expression in vivo, C57BL/6 mice (Jackson laboratories Germany) were injected with 0.5 mg/kg Firefly luciferase mRNA (TriLink, CleanCap, 5 moU), as loaded in aNPs containing polyvalent molecule A2 mRNA binder. After 16 hours, the animals were sacrificed and the liver, spleen and femur collected. Organs were processed to yield single cell suspensions. 1/10th of the liver was cut into small pieces, incubated for 15 min, 37° C., 50 RPM with 5 mL liberase solution. Next, the digested liver was put through a 70 μm strainer and collected in a 50 mL falcon tube. After washing with 1×PBS, the cells was resuspended in 15 mL 1×PBS. 1/10th of the spleen was cut into small pieces and put through a 70 μm strainer and collected in a 50 mL falcon tube. After washing with 1×PBS, 1 mL of lysis buffer (BD Pharm Lyse™, cat #555899, diluted 10 times in diH2O) was added and incubated for 4 minute at room temperature. The cells were washed with 1×PBS and resuspended in 15 mL 1×PBS. Both ends of the femur were broken. The bone marrow cells were flushed from the inside of the bone with 10 mL 1×PBS through a 70 μm strainer and collected in a 50 mL falcon tube. The cells were centrifugated and 1 mL lysis buffer (BD Pharm Lyse™, cat #555899, diluted 10 times in diH2O) was added to the pellet and incubated for 1 minute at room temperature. The cells were washed with 1×PBS and resuspended in 5 mL 1×PBS. All cell suspensions were counted and 500000 cells were seeded in a V-bottom plate. The plate was centrifugated and the supernatant removed. 40 μL of the ONE-Glo reagent (Promega) was added and incubated for 5 minutes (432 RPM). 40 μL was transferred to a white 96-well plate and luminescence was measured using the following settings: Settle time: 1000 ms; Integration time: 500 ms; Open filter. Results are reported in FIG. 16.in vitroin vitro

1. A nanoparticle comprising a core and an outer layer, wherein the core comprises:
a nucleic acid;
a polyvalent molecule;
and wherein the outer layer comprises:
an apolipoprotein, an apolipoprotein derivative, an apolipoprotein mimetic and/or an apolipoprotein mimetic derivative;
a phospholipid;
a sterol; and optionally
a filler molecule,
wherein the polyvalent molecule has formula (I): 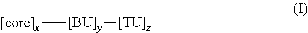
wherein the core is a nitrogen or is a C₁-C₁₈ linear, branched, or cyclic group that contains 1 to 15 nitrogen heteroatoms and that optionally contains 1 to 4 oxygen heteroatoms; and
wherein x represents the number of connections from the core to the branching units BU (or to the terminal units TU, when y=0), where x is 1, 2, 3, 4, 5, 6, 7, 8, 9, 10 or 12, and where these connections are all made from nitrogen atoms of the core to carbon atoms of the branching unit BU (or of the terminal unit TU); and
wherein the branching unit BU has formula (IIa), (IIb), (IIc) or (IId): 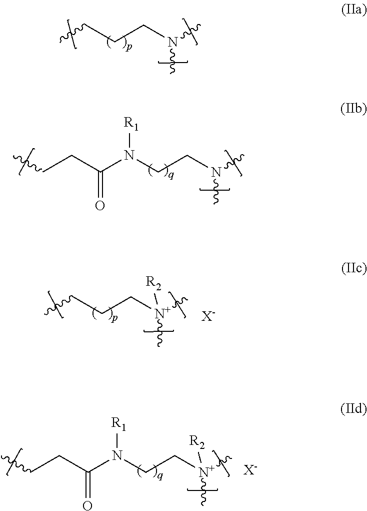
wherein (IIa) represents an n-alkylene-nitrogen spacer that is connected at the CH₂-end to the core or to a BU that is closer to the core, and that is connected at the N-end to two terminal units (TUs) or to two branching units (BUs) that are closer to the TUs, where the connections are given in wavy bonds, and wherein p is 1, 2, 3 or 4; and
wherein (IIb) represents an amide containing n-alkylene-nitrogen spacer that is connected at the CH₂-end to the core or to a BU that is closer to the core, and that is connected at the N-end to two terminal units (TUs) or to two branching units (BUs) that are closer to the TUs, where the connections are given in wavy bonds, wherein q is 1, 2, 3, 4 or 5, and wherein R₁ is a hydrogen, a methyl, an ethyl, an n-propyl or an iso-propyl group; and
wherein (IIc) and (IId) are defined as (IIa) and (IIb), respectively, and wherein R₂ is a methyl, ethyl, n-propyl, benzyl, acetamide (—CH₂—C(O)NH₂) or 2-hydroxy-ethylene group, and wherein X⁻ is the counter anion to the quaternary amine cation moiety; and
wherein y represents the specific and discrete generation number of the polyvalent molecule, where this number indicates how many successive BU layers are incorporated in the polyvalent molecule, as counted from traveling from the inner core to the outer TU groups, and where y is 0, 1, 2, 3, 4 or 5; and
wherein for each separate generational layer the BU can independently be selected from formulas (IIa), (IIb), (IIc) or (IId), i.e. the first BU emanating from the core, representing the first generational layer BU, can be different from the second BU emanating further away from the core (representing the second generational layer BU), and this one may be different from the third, the fourth and the fifth BU emanating further away from the core; and
wherein the terminal units (TU) are individually and independently chosen from a hydrogen and a C₁-C₃₀ alkyl, aryl, arylene-alkyl or alkylene-aryl group that optionally contains 1 to 8 heteroatoms that are individually and independently selected from the group consisting of O and N, with the proviso that not all TUs in the polyvalent molecule are hydrogens; and
wherein z represents the total number of TU groups that are attached to the polyvalent molecule, where z is 1 to 128; and
wherein the polyvalent molecule as according to formula (I) is polyvalent in positively ionizable and/or cationic groups, where the cumulative number of positively ionizable and cationic groups in the polyvalent molecule is 2 or higher.

2. The nanoparticle according to claim 1, wherein the polyvalent molecule has formula (I): 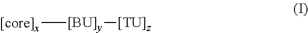
wherein y=0, 1, 2, or 3.

3. The nanoparticle according to claim 1, wherein the polyvalent molecule is a first, second or third generation dendrimer selected from a poly(propylene imine) (PPI) dendrimer or a polyamidoamine (PAMAM) dendrimer, most preferably a PPI dendrimer, or a modification thereof.

4. The nanoparticle according to claim 1, wherein the polyvalent molecule has a structure selected from: 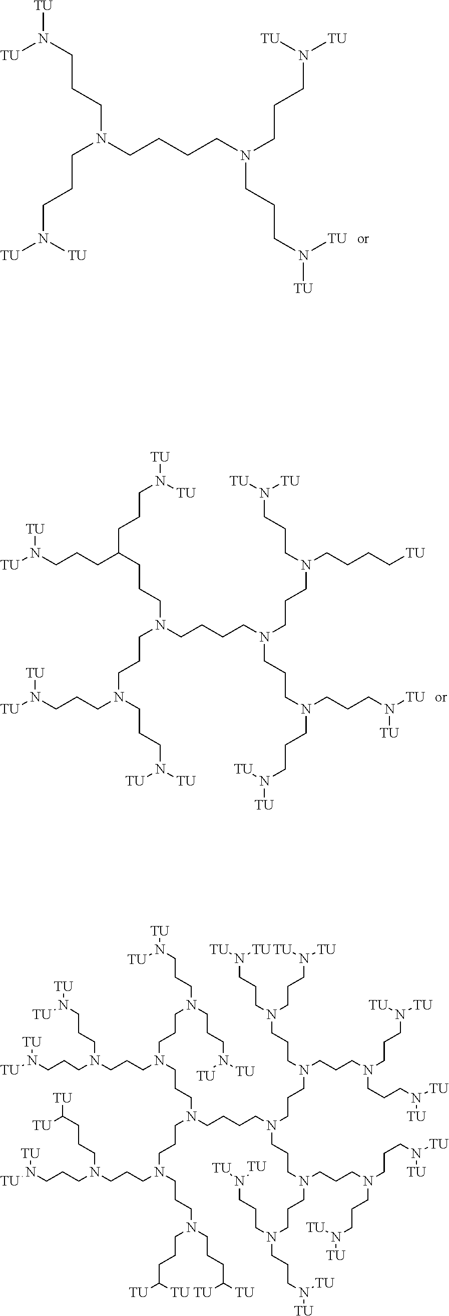
wherein the terminal units (TU) are individually and independently chosen from a hydrogen and a C₁-C₃₀ alkyl, aryl, arylene-alkyl or alkylene-aryl group that optionally contains 1 to 8 heteroatoms that are individually and independently selected from the group consisting of O and N, with the proviso that not all TUs in the polyvalent molecule are hydrogens.

5. The nanoparticle according to claim 1, wherein the apolipoprotein, apolipoprotein derivative, apolipoprotein mimetic or apolipoprotein mimetic derivative is selected form apo A1, apo A1-Milano, apo A2, apo A4, apo A5, apo B48, apo B100, apo C-I, apo C-II, apo C-III, apo C-IV, apo D, apo E, apo F, apo H, apo L and apo M or a mimetic or derivatives thereof,
preferably selected from apo A1, apo A2, apo A4, apo A5, apo B100, apo C-I, apo C-II, apo C-III, apo C-IV and apo E or a mimetic or derivatives thereof,
more preferably selected from apo A1, apo A4, apo A5, apo B100, apo C-III and apo E or a mimetic or derivatives thereof,
most preferably selected from apo A1, apo B100 and apo E or a mimetic or derivatives thereof.

6. The nanoparticle according to claim 1, wherein the nucleic acid is RNA, DNA, or a nucleic acid analogue,
preferably wherein the RNA is microRNA (miRNA), small interfering RNA (siRNA), piwi-interacting RNA (piRNA), small nuclear RNA (snoRNA), transfer RNA (tRNA), tRNA-derived small RNA (tsRNA), small regulatory RNA (srRNA), messenger RNA (mRNA), modified mRNA, ribosomal RNA (rRNA), self-amplifying RNA (saRNA), circular RNA (circRNA), long non-coding RNA (lncRNA), or guide RNA (gRNA) or combinations thereof and/or modifications thereof, or
preferably wherein the DNA is single stranded or double stranded DNA; or
preferably wherein the nucleic acid is an antisense oligonucleotide which is single stranded DNA or RNA consisting or comprising of nucleotide or nucleoside analogues containing modifications of the phosphodiester backbone or the 2′ ribose, more preferably wherein the nucleotide or nucleoside analogues are selected from locked nucleic acid (LNA), bridged nucleic acid (BNA), morpholino or peptide nucleic acid (PNA).

7. The nanoparticle according to claim 1, wherein the sterol is preferably selected from sterol, cholesterol, ergosterol, hopanoids, hydroxysteroid, phytosterol, steroids, zoosterol, stigmasterol, or β-sitosterol, or combinations thereof.

8. The nanoparticle according ton claim 1, wherein:
the phospholipid is selected from a phosphatidylcholine, a phosphatidylethanolamine, a phosphatidylserine and a phosphatidylglycerol or combinations thereof, preferably wherein at least one, more preferably both, of the acyl groups in the phospholipid are derived from long chain fatty acids, even more preferably wherein said long chain fatty acids are selected from lauric acid, lauroleic acid, myristic acid, myristoleic acid, palmitic acid, palmitoleic acid, stearic acid, oleic acid and linoleic acid, or combinations thereof.

9. The nanoparticle according to claim 1, further comprising a filler molecule, preferably wherein the filler molecule is a glyceride molecule, more preferably wherein the filler molecule is selected from a triglyceride, a modified triglyceride, and a cholesteryl ester, or combinations thereof, preferably wherein the triglyceride is derived from C6-C18 fatty acids, preferably tricaprylin and/or wherein the cholesteryl ester is cholesteryl acetate, cholesteryl caprylate and/or cholesteryl oleate.

10. The nanoparticle according to claim 1, wherein:
the amount of apolipoprotein, apolipoprotein derivative, apolipoprotein mimetic or apolipoprotein mimetic derivative ranges from 0.1 to 90 weight %; and/or
the amount of nucleic acid ranges from 0.01 to 90 weight %;
the amount of phospholipid ranges from 0.1 to 95 weight %; and/or
the amount of sterol ranges from 0.1 to 95 weight %; and/or
the amount of polyvalent molecule ranges from 0.1 to 95 weight %,
the amount of optionally present filler comprises ranges from 0 to 95 weight %,
wherein the weight percentages are based on the combined amounts of these five components plus the optional sixth filler component, i.e. these five or six components add up to 100% of the weight of the nanoparticle.

11. A composition comprising the nanoparticle according to claim 1 and a physiologically acceptable carrier, preferably wherein the composition is a pharmaceutical composition.

12. (canceled)

13. A method of delivering a nucleic acid to the myeloid compartment or the spleen in a subject in need thereof, comprising administering the composition according to claim 11 to the subject.

14. A method of treating a disease in a subject in need thereof, comprising administering the composition according to claim 11 to the subject, thereby stimulating or inhibiting an innate immune response in the subject.

15. The method according to claim 14, wherein said disease is a cancer, a cardiovascular disease, an autoimmune disorder or xenograft rejection.

16. A method for producing a nanoparticle, comprising the step of:
a) mixing, preferably rapid mixing, of (lipid) components in organic solvent with a nucleic acid in an aqueous buffer to produce nanoparticles, wherein the (lipid) components comprise a phospholipid, a sterol, a polyvalent molecule, and optionally a filler molecule; and wherein the aqueous buffer has a pH of 5.5 or lower, preferably 5.0 or lower; and
b) mixing, preferably rapid mixing, of lipid nanoparticles with an apolipoprotein, an apolipoprotein derivative, an apolipoprotein mimetic and/or an apolipoprotein mimetic derivative, to produce the nanoparticle at a pH between 5.5 and 9.0, preferably at a pH between 6.0 and 8.0, more preferably at a pH between 6.5 and 8.0.

17. A nanoparticle that is obtainable or obtained by the method of claim 16.

18. An in vitro or ex vivo method for introducing a nucleic acid in a cell, the method comprising contacting the nanoparticle according to claim 1 with a cell.

19. An in vivo method for introducing a nucleic acid in a cell, the method comprising contacting the nanoparticle according to claim 1 with a cell.

20. (canceled)

21. A method for the in vivo delivery of a nucleic acid, the method comprising administering the composition according to claim 11 to a subject.

22. A method for stimulating or inhibiting an innate immune response in a subject in need thereof, the method comprising administering a composition according to claim 11 to the subject.

23. The method according to claim 22, wherein the subject has a disease selected from cancer, cardiovascular disease, autoimmune disorder or xenograft rejection.


SEQUENCE LISTING (1 sequence)

SEQ ID NO: 1 - protein, 18 aa, synthetic construct
DWLKAFYDKVAEKLKEAF